(19) United States
(12) Patent Application Publication
(10) Pub. No.: US 2009/0181968 A1
(43) Pub. Date: Jul. 16, 2009

(54) Novel 3-Bicyclocarbonylaminopyridine-2-Carboxamides or 3-Bicyclocarbonylaminopyrazine-2-Carboxamides

(75) Inventors: Kosrat Amin, Molndal (SE); Johan Broddefalk, Molndal (SE); Yantao Chen, Molndal (SE); Karolina Nilsson, Molndal (SE); Claire Milburn, Thousand Oaks, CA (US); Helene Desfosses, Montreal (CA); Ziping Liu, Montreal (CA); Maxime Tremblay, Montreal (CA); Christopher Walpole, Montreal (CA); Zhong-yong Wei, Montreal (CA); Hua Yang, Verdun (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/094,334

(22) PCT Filed: Nov. 22, 2006
(86) PCT No.: PCT/SE06/01326

§ 371 Date: Dec. 4, 2008

(30) Foreign Application Priority Data
Nov. 24, 2005 (SE) 0502578-8

(51) Int. Cl. A61K 31/5377; C07D 401/10; C07D 405/14; A61K 31/4439; A61K 31/497; C07D 413/14; C07D 405/12; A61K 31/4433; A61K 31/496; C07D 401/12; A61K 31/4709; C07D 409/14; A61K 31/4436; C07D 417/12; A61K 31/4375; C07D 413/12; A61P 29/00

(52) U.S. Cl. 514/236.2; 546/268.4; 514/340; 544/405; 514/255.05; 544/131; 546/282.1; 514/336; 544/364; 514/253.11; 514/339; 546/278.1; 546/169; 514/314; 514/338; 546/275.7; 546/281.1; 514/337; 546/270.1; 546/123; 514/300; 546/272.1

(57) ABSTRACT

The present invention relates to compounds of formula (I) [Chemical formula should be inserted here. Please see paper copy] as well as pharmaceutically acceptable salts and pharmaceutical compositions including the compounds are prepared or thereof: wherein, A¹, A², R¹, R², R³, R⁴, and R⁵ and n are as defined in the specification. The compounds of formula (I) are useful in therapy.

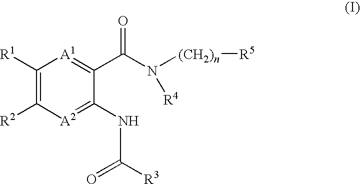

### FIELD OF THE INVENTION

The present invention is related to new compounds, pharmaceutical compositions containing these compounds, manufacturing processes and uses thereof. The present invention is also related to compounds which may be effective in treating pain, cancer, multiple sclerosis, Parkinson's disease, Huntington's chorea, Alzheimer's disease, anxiety disorders, gastrointestinal disorders and/or cardiovascular disorders.

### BACKGROUND OF THE INVENTION

It is known that cannabinoid receptor (e.g., CB₁ receptor, CB₂ receptor) ligands including agonists, antagonists and inverse agonists produce relief of pain in a variety of animal models by interacting with CB₁ and/or CB₂ receptors. Generally, CB₁ receptors are located predominately in the central nervous system, whereas CB₂ receptors are located primarily is in the periphery and are primarily restricted to the cells and tissues derived from the immune system.

While CB₁ receptor agonists, such as Δ⁹-tetrahydrocannabinol (Δ⁹-THC) and anadamide, are useful in anti-nociception models in animals, they tend to exert undesired CNS side effects, e.g., psychoactive side effects, the abuse potential, drug dependence and tolerance, etc. These undesired side effects are known to be mediated by the CB₁ receptors located in CNS. There are lines of evidence, however, suggesting that CB1 agonists acting at peripheral sites or with limited CNS exposure can manage pain in humans or animals with much improved overall in vivo profile.

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastroesophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD. GERD is caused by reflux of gastric contents into the esophagus leading to heartburn and other typical symptoms. In many cases, an inflammation develops in the distal esophagus (esophagitis). It has been known for a long time that suppression of production of gastric acid ameliorates both symptoms and esophagitis. However, some patients continue to have symptoms despite adequate control of acid secretion. Reflux of other noxious factors is believed to be responsible for those symptoms. Most focus has been centered on the importance of bile acids, and the development of severe GERD is related to the degree of esophageal bile acid exposure.

There is a need for new CB₁ receptor ligands such as agonists that may be useful in is managing gastrointestinal disorders or pain or treating other related symptoms or diseases with reduced or minimal undesirable CNS side effects.

### OUTLINE OF THE INVENTION

The present invention provides CB₁ receptor ligands, which may be useful in treating pain, cancer, multiple sclerosis, Parkinson's disease, Huntington's chorea, Alzheimer's disease, anxiety disorders, gastrointestinal disorders and/or cardiovascular disorders.

The present invention relates to a compound of formula (I)

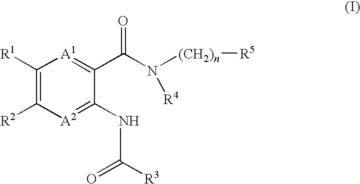

wherein:
at least one of A¹ and A² is N and if both are not N, then the other is CH;
R¹ is selected from hydrogen, cyano, halogen, hydroxy, NR⁶R⁷, C₂₋₆alkenyl, C₂₋₆alkynyl, C₁₋₉alkyl, C₃₋₆cycloalkyl and C₁₋₆haloalkoxy, wherein said C₂₋₆alkenyl, C₂₋₆alkynyl, C₁₋₉alkyl, C₃₋₆cycloalkyl or C₁₋₆haloalkoxy is optionally substituted by hydroxy, NR^{6a}R^{7a}, C₃₋₆cycloalkyl, aryl and heteroaryl;
R² is selected from hydrogen, cyano, halogen, hydroxy, NR⁶R⁷, C₂₋₆alkenyl, C₂₋₆alynyl, C₁₋₆alkyl, C₃₋₆cycloalkyl and C₁₋₆haloalkoxy, wherein said C₂₋₆alkenyl, C₂₋₆alkynyl, C₁₋₆alkyl, C₃₋₆cycloalkyl or C₁₋₆haloalkoxy is optionally substituted by hydroxy, NR^{6a}R^{7a}, C₃₋₆cycloalkyl, aryl and heteroaryl;
R³ is selected from

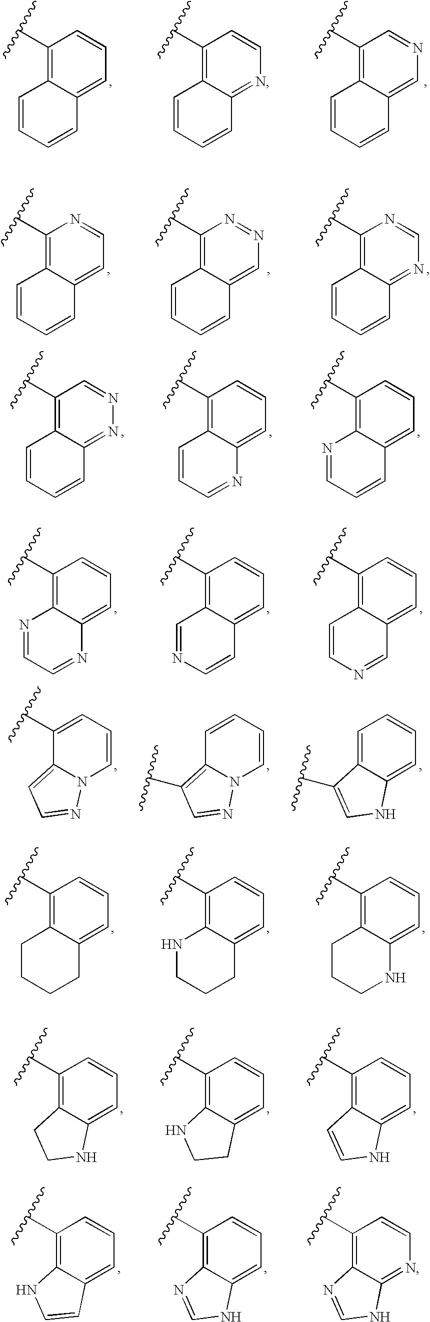

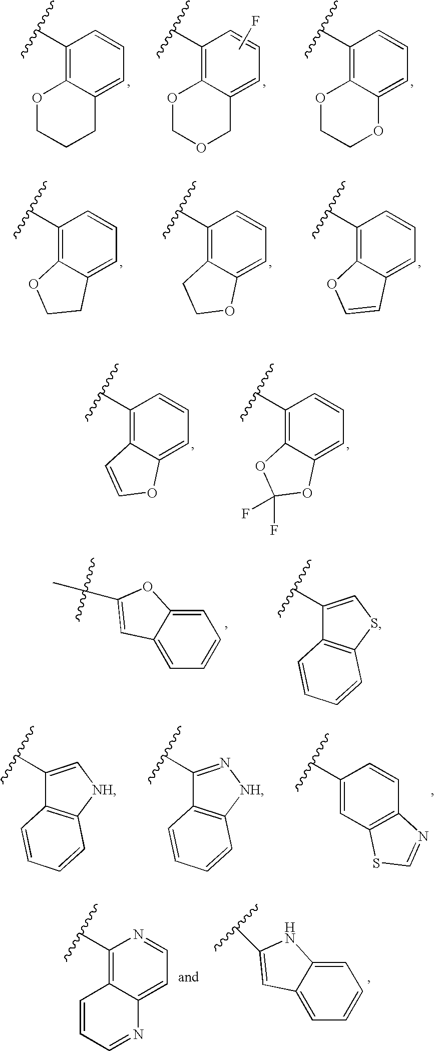

and wherein R³ is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl, wherein said C₁₋₆alkyl, C₃₋₆cycloalkyl, aryl or heteroaryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl, heteroaryl or a saturated ring system consisting of 4 to 7 atoms selected from C, N and O, and wherein said C₁₋₆alkyl, C₃₋₆cycloalkyl, aryl, heteroaryl or ring system is optionally substituted by C₁₋₄alkyl, and wherein said C₁₋₄alkyl is optionally substituted by NR⁶R⁷, aryl, hydroxy or C₁₋₄alkoxy;
R⁴ is selected from hydrogen and C₁₋₆alkyl;
R⁵ is selected from C₁₋₆alkyl, C₃₋₆cycloalkyl, C₁₋₆alkoxy, C₁₋₆haloalkoxy, heteroaryl and aryl, wherein said C₁₋₆alkyl, C₃₋₆cycloalkyl, heteroaryl or aryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
n is selected from 0, 1, 2, 3, 4 and 5;
or R⁴ and R⁵ together form a saturated, unsaturated or partly saturated ring system consisting of 3 to 7 atoms selected from C, O and N;
or R⁴ and R⁵ together form a saturated, unsaturated or partly saturated condensed ring system consisting of 7 to 13 atoms selected from C, O and N;
wherein said ring system is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl, and wherein said C₁₋₆alkyl, C₃₋₆cycloalkyl, aryl or heteroaryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
R⁶, R^{6a}, R⁷ and R^{7a} are each and independently selected from hydrogen, C₁₋₆alkyl, C₃₋₆cycloalkyl, C₁₋₆alkoxy, C₁₋₆haloalkoxy, C₂₋₆alkenyl, C₂₋₆alkynyl, aryl and heteroaryl; or
R^{6a} and R^{7a} may together form a saturated ring system consisting of 4 to 7 atoms selected from C, O and N, which ring system is optionally substituted with C₁₋₆alkyl, C₁₋₆alkoxy, halogen or hydroxy;
wherein one or more carbon atom(s) of each alkyl or cycloalkyl group as defined for R¹, may be substituted for O, NH, C(O), SO or SO₂, wherein none of the N or O is in a position adjacent to any other O or N and wherein none of the SO or SO₂ is in a position adjacent to any other SO or SO₂;
wherein one or more carbon atom(s) of each alkyl or cycloalkyl group as defined for R², R³, R⁴, R⁵, R⁶, R^{6a}, R⁷ and R^{7a} may be substituted for O, NH, C(O) or SO₂, wherein none of the N or O is in a position adjacent to any other O or N;
wherein one or more carbon atom(s) of each alkyl or cycloalkyl group as defined for R¹, R², R³, R⁴, R⁵, R⁶, R^{6a}, R⁷ and R^{7a} may be substituted by fluoro; and
with the proviso that R¹ is not hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl or NR⁶R⁷; at the same time as R² is hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl or NR⁶R⁷; unless R³ is substituted by a C₁₋₄ alkyl, which C₁₋₄alkyl is substituted by a heteroaryl, C₃₋₆cycloalkyl, aryl or a saturated ring system consisting of 4 to 7 atoms selected from C, O and N, wherein said heteroaryl, C₃₋₆cycloalkyl or aryl is further substituted by C₁₋₄alkyl or halogen, wherein said C₁₋₄alkyl is optionally substituted by NR⁶R⁷, aryl, hydroxy or C₁₋₄alkoxy, and wherein said ring system is optionally substituted by C₁₋₄alkyl, wherein said C₁₋₄ alkyl is optionally substituted by NR⁶R⁷, aryl, hydroxy or C₁₋₄alkoxy; is or unless R³ is selected from:

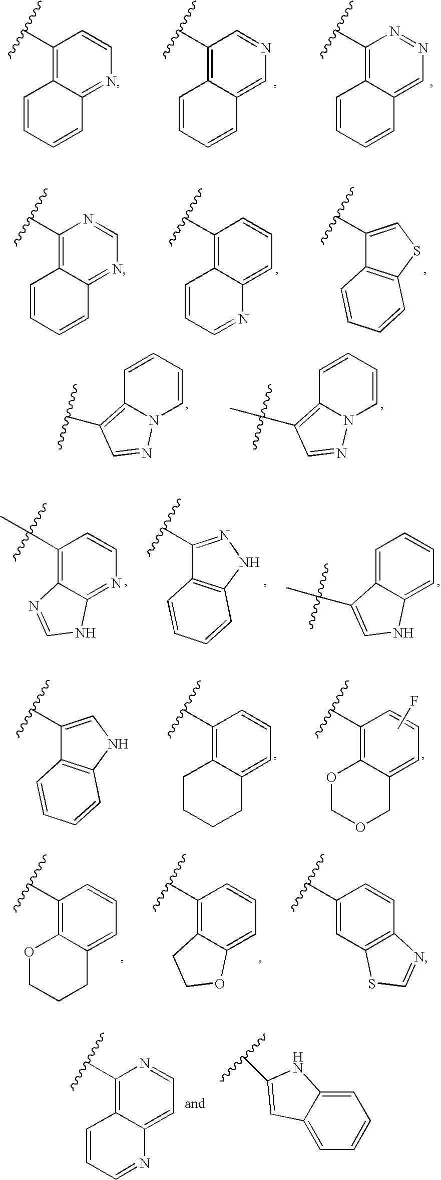

optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
or a pharmaceutically acceptable salt thereof, or diastereomers, or enantiomers, or mixtures thereof.

The present invention also relates to a compound of formula (I)

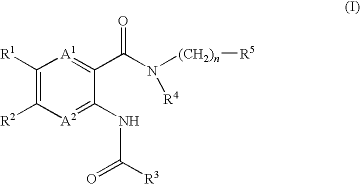

wherein:
at least one of A¹ and A² is N and if both are not N then the other is CH;
R¹ is selected from hydrogen, cyano, halogen, hydroxy, NR⁶R⁷, C₂₋₆alkenyl, C₂₋₆alkynyl, C₁₋₆alkyl, C₃₋₆cycloalkyl and C₁₋₆haloalkoxy, wherein said C₂₋₆alkenyl, C₂₋₆alkynyl, C₁₋₆alkyl, C₃₋₆cycloalkyl or C₁₋₆haloalkoxy is optionally substituted by hydroxy, NR^{6a}R^{7a}, C₃₋₆cycloalkyl, aryl or heteroaryl;
R² is selected from hydrogen, cyano, halogen, hydroxy, NR⁶R⁷, C₂₋₆alkenyl, C₂₋₆alkynyl, C₁₋₆alkyl, C₃₋₆cycloalkyl and C₁₋₆haloalkoxy, wherein said C₂₋₆alkenyl, C₂₋₆alkynyl, C₁₋₆alkyl, C₃₋₆cycloalkyl or C₁₋₆haloalkoxy is optionally substituted by hydroxy, NR^{6a}R^{7a}, C₃₋₆cycloalkyl, aryl or heteroaryl;
R³ is selected from

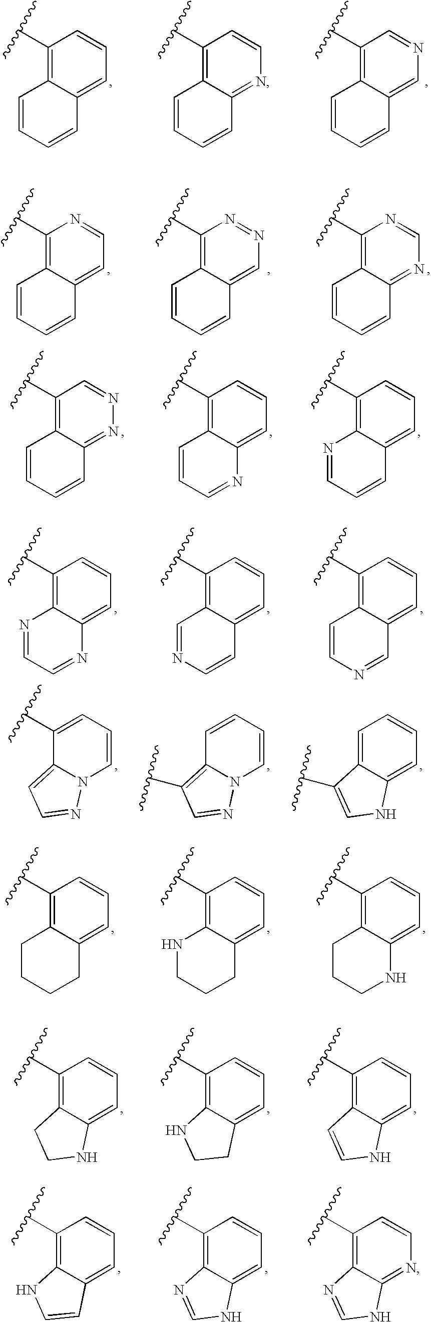

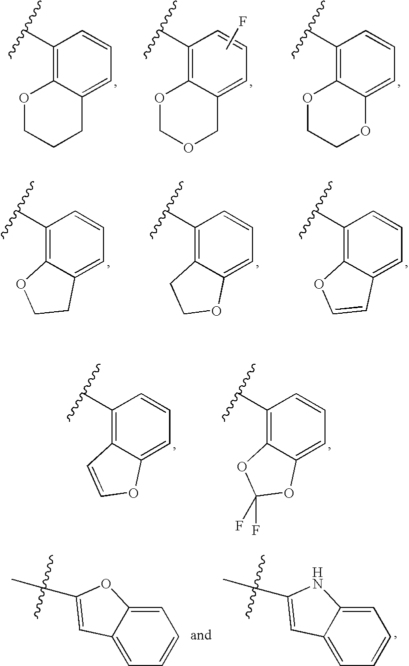

and wherein R³ is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl, wherein said C₁₋₆alkyl, C₃₋₆cycloalkyl, aryl or heteroaryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl; and wherein said C₁₋₆alkyl, C₃₋₆cycloalkyl, aryl or heteroaryl is optionally substituted by C₁₋₄alkyl and wherein said C₁₋₄alkyl is optionally substituted by NR⁶R⁷, aryl, hydroxy or C₁₋₄alkoxy;
R⁴ is selected from hydrogen and C₁₋₆alkyl;
R⁵ is selected from C₁₋₆alkyl, C₃₋₆cycloalkyl, C₁₋₆alkoxy, C₁₋₆haloalkoxy, heteroaryl and aryl, wherein said C₁₋₆alkyl, C₃₋₆cycloalkyl, heteroaryl or aryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
n is selected from 0, 1, 2, 3, 4 and 5;
or R⁴ and R⁵ together form a saturated, unsaturated or partly saturated ring system consisting of 3 to 7 atoms selected from C, O and N;
or R⁴ and R⁵ together form a saturated, unsaturated or partly saturated condensed ring system consisting of 7 to 13 atoms selected from C, O and N;
wherein said ring system is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl, and wherein said C₁₋₆alkyl, C₃₋₆cycloalkyl, aryl or heteroaryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
R⁶, R^{6a}, R⁷ and R^{7a} are each and independently selected from hydrogen, C₁₋₆alkyl, C₃₋₆cycloalkyl, C₁₋₆alkoxy, C₁₋₆haloalkoxy, C₂₋₆alkenyl, C₂₋₆alkynyl, aryl and heteroaryl;
wherein one or more carbon atom(s) of each alkyl or cycloalkyl group as defined for R¹, R², R³, R⁴, R⁵, R⁶, R^{6a}, R⁷ and R^{7a} may be substituted for O, NH, C(O) or SO₂;
wherein one or more carbon atom(s) of each alkyl or cycloalkyl group as defined for R¹, R², R³, R⁴, R⁶, R^{6a}, R⁷ and R^{7a} may be substituted by fluoro; and
with the proviso that R¹ is not hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl or NR⁶R⁷; at the same time as R² is hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl or NR⁶R⁷; unless R³ is substituted by a C₁₋₄ alkyl, which C₁₋₄alkyl is substituted by a heteroaryl, C₃₋₆cycloalkyl or aryl that is further substituted by C₁₋₄alkyl wherein said C₁₋₄alkyl is optionally substituted by NR⁶R⁷, aryl, hydroxy or C₁₋₄alkoxy; or unless R³ is selected from:

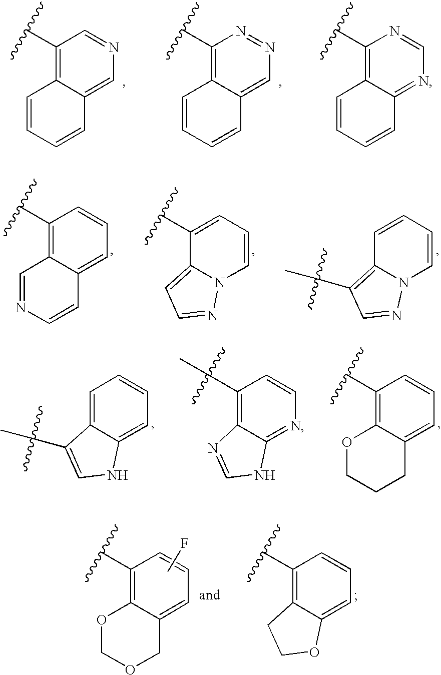

or a pharmaceutically acceptable salt thereof, or diastereomers, or enantiomers, or mixtures thereof.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein. The IUPAC names have been obtained by ACD/Labs Name (version 9.04 2005).

The term “Cₘ₋ₙ” or “Cₘ₋ₙ group” used alone or as a prefix, refers to any group having m to n carbon atoms.

The term “C₁₋₉alkyl” includes linear or branched C₁₋₉alkyl. Examples of C₁₋₉alkyl include, but are not limited to, methyl, ethyl, propyl, n-propyl, isopropyl, butyl, iso-butyl, sec-butyl, tert-butyl, hexyl, octyl, nonyl and decyl.

The term “C₁₋₆alkyl” includes linear or branched C₁₋₆alkyl. Examples of C₁₋₆alkyl include, but are not limited to, methyl, ethyl, propyl, n-propyl, isopropyl, butyl, iso-butyl, sec-butyl, tert-butyl and hexyl.

The term “C₁₋₄alkyl” includes linear or branched C₁₋₄ alkyl. Examples of C₁₋₄alkyl include, but are not limited to, methyl, ethyl, propyl, n-propyl, isopropyl, butyl, iso-butyl, sec-butyl, and tert-butyl.

The term “C₃-C₆cycloalkyl” is intended to include monovalent rings having from 3 up to 6 carbons. Examples of such rings are cyclopropyl, cyclobutyl, cyclopentyl, and cyclohexyl.

The term “C₁₋₆alkoxy” includes linear or branched C₁₋₆alkoxy. Examples of C₁₋₆alkoxy include, but are not limited to, methoxy, ethoxy, n-propanoxy, isopropanoxy, isobutoxy, secondary butoxy and hexoxy.

The term “C₁₋₄alkoxy” includes linear or branched C₁₋₄alkoxy. Examples of C₁₋₄alkoxy include, but are not limited to, methoxy, ethoxy, n-propanoxy, isopropanoxy, isobutoxy, and secondary butoxy.

The term “aryl” means an aromatic ring having 6-14 carbons including both single rings and polycyclic compounds. Examples of such rings include, but are not limited to, phenyl, benzyl and naphthyl.

The term “heteroaryl” as used herein means a heteroaromatic ring having 3-14 carbon atoms, in which one or more of the ring atoms is either oxygen or nitrogen or sulphur including both single rings and polycyclic compounds. For example a five-membered ring heteroaryl is a heteroaryl with a ring having five ring atoms wherein 1, 2 or 3 ring atoms are independently selected from N, O and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered ring heteroaryl is a heteroaryl with a ring having six ring atoms wherein 1, 2 or 3 ring atoms are independently selected from N, O and S. Exemplary six-membered ring heteroaryls are pyridyl, pyrazinyl, pyrimidinyl, triazinyl and pyridazinyl.

The term “C₂₋₆alkenyl” as used herein is intended to refer to a monovalent straight or branched chain hydrocarbon radical having at least one-carbon-carbon double bond and comprising at least 2 up to 6 carbon atoms.

The term “C₂₋₆alkynyl” as used herein is intended to refer to a monovalent straight or branched chain hydrocarbon having at least one carbon-carbon triple bond and comprising at least 2 up to 6 carbon atoms.

The term “halogen” as used herein is intended to include fluorine, bromine and iodine.

“Halo” as used herein as a prefix means that one or more hydrogens on a group has been replaced with one or more halogens.

The term “nitro” as used herein is intended to refer to a NO₂-group.

The following aberrations are used in the present application:
Acetylamino CH₃CONH;
Ar aryl;
CH₂Cl₂ dichloromethane;
CH₃COOH acetic acid;
CH₃CN acetonitrile;
CHCl₃ trichloromethane;
CCl₄ tetrachloromethane;
DCM dichloromethane;
DIPEA N,N-diisopropylethylamine;
DMAP 4-dimethylamino pyridine;
DMF dimethyl formamide;
EDTA ethylenediaminetetraacetic acid;
Et₃N triethyl amine;
EtOAc ethyl acetate;
EtOH ethanol;
KCN potassium cyanide;
K₂CO₃ potassium carbonate;
HCl hydrochloric acid;
MeOH methanol;
MeCN acetonitrile;
MgSO₄ magnesium sulphate;
MTBE methyl tert-butyl ether;
NBS N-bromosuccinimide;
NaH sodium hydride;
NH₄Cl ammonium chloride;
NaOH sodium hydroxide;
NaHCO₃ sodium bicarbonate;
Na₂SO₄ sodium sulphate;
Pd(AcO)₂ palladium diacetate;
Pd/C palladium on carbon;
RT room temperature;
TBTU benzotriazol-1-yl-N-tetramethyl-uronium tetrafluoroborate;
TFA trifluoroacetic acid;
TMEDA N,N,N′N′-tetramethyl 1,2-ethanediamine;
TMSCl trimethylsilyl chloride.

In one embodiment of the present invention A¹ and A² are N. According to another embodiment of the present invention A¹ is N and A² is CH.

In another embodiment of the present invention R⁴ is hydrogen.

In a further embodiment of the present invention n is 1.

Yet in a further embodiment of the present invention R¹ is hydrogen.

According to another embodiment of the present invention, R² is C₁₋₆alkyl, optionally substituted by hydroxy, NR^{6a}R^{7a}, C₃₋₆cycloalkyl, aryl or heteroaryl.

Yet in a further embodiment of the present invention, R¹ is hydrogen.

According to yet another embodiment of the present invention, R¹ is selected from cyano, halogen, NR⁶R⁷, C₁₋₉alkyl, C₃₋₆cycloalkyl and C₁₋₆haloalkoxy and wherein said C₁₋₉alkyl, C₃₋₆cycloalkyl or C₁₋₆haloalkoxy is optionally substituted by hydroxy, NR^{6a}R^{7a}, C₃₋₆cycloalkyl, aryl or heteroaryl.

According to another embodiment of the present invention, R¹ is selected from cyano, halogen, NR⁶R⁷, C₁₋₆alkyl, C₃₋₆cycloalkyl and C₁₋₆haloalkoxy, wherein said C₁₋₆alkyl, C₃₋₆cycloalkyl or C₁₋₆haloalkoxy is optionally substituted by hydroxy, NR^{6a}R^{7a}, C₃₋₆cycloalkyl, aryl or heteroaryl.

In another embodiment of the present invention R¹ is C₁₋₉alkyl, optionally substituted by hydroxy, NR^{6a}R^{7a}, C₃₋₆cycloalkyl, aryl or heteroaryl.

In yet another embodiment of the present invention R¹ is C₁₋₆alkyl, optionally substituted by hydroxy, NR^{6a}R^{7a}, C₃₋₆cycloalkyl, aryl or heteroaryl.

According to one embodiment of the present invention one carbon atom of the alkyl as defined for R¹ is substituted by at least one fluoro. According to another embodiment of the present invention wherein at least one carbon atom of the alkyl group as defined for R¹ is substituted for O. In yet another embodiment of the present invention at least one carbon atom of the alkyl group as defined for R¹ is substituted for NH, C(O), SO or SO₂. In a further embodiment of the present invention R¹ is C₃₋₉alkyl and at least two carbon atoms of the alkyl group as defined for R¹ is substituted for O. In yet a further embodiment of the present invention R¹ is C₃₋₆alkyl and at least two carbon atoms of the alkyl group as defined for R¹ is substituted for O. In another embodiment of the present invention at least one carbon atom of the alkyl group as defined for R¹ is substituted for C(O).

According to one embodiment of the present invention, R¹ is C₃₋₆cycloalkyl. According to another embodiment of the present invention, R⁵ is C₄cycloalkyl or C₆cycloalkyl.

According to a further embodiment of the present invention, R⁵ is cyclobutyl or cyclohexyl or tetrahydropyran.

In another embodiment of the present invention R³ is selected from

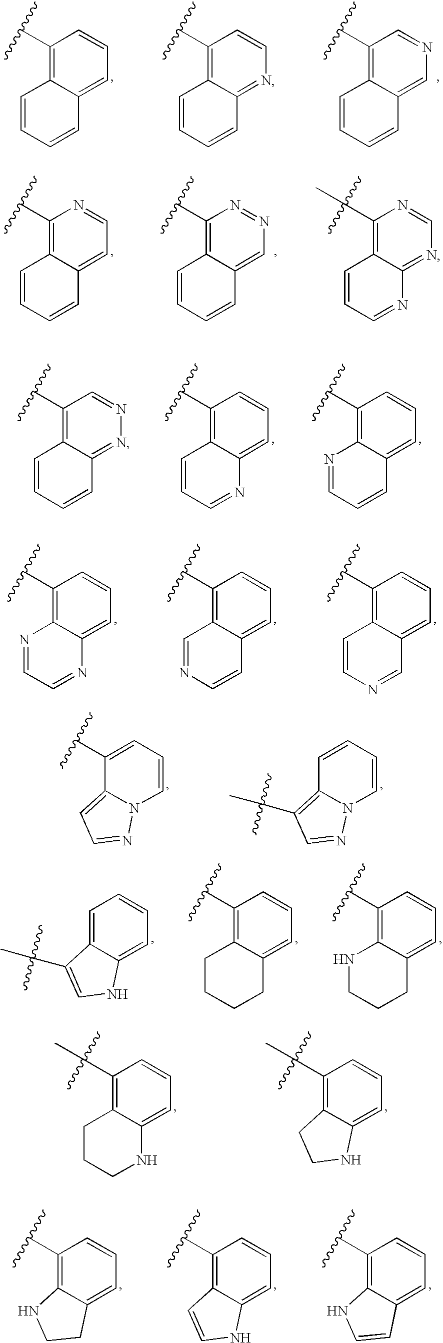

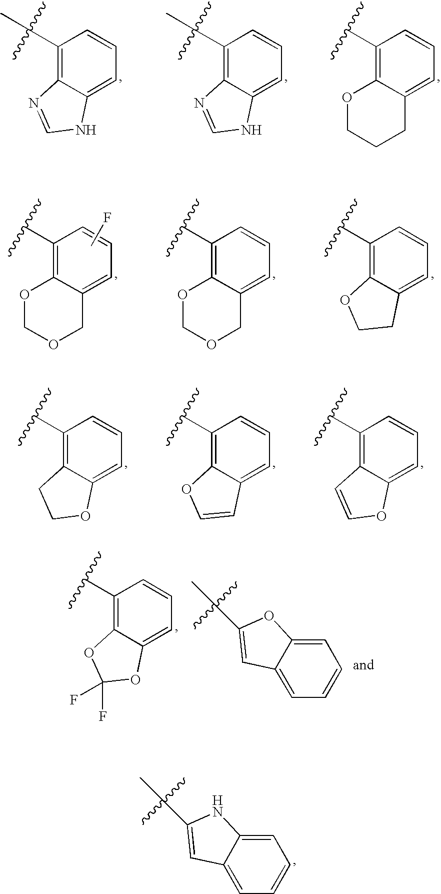

and wherein R³ is optionally substituted by halogen, C₁₋₆alkyl, C₃₋₆cycloalkyl, C₁₋₆alkoxy or C₁₋₆haloalkoxy, wherein said C₁₋₆alkyl or C₃₋₆cycloalkyl is optionally substituted by halogen, NR⁶R⁷, C₁₋₆alkyl, C₃₋₆cycloalkyl, C₁₋₆alkoxy, aryl, heteroaryl or a saturated ring system consisting of 4 to 7 atoms selected from C, N and O, and wherein said C₁₋₆alkyl, C₃₋₆cycloalkyl, aryl, heteroaryl or ring system is optionally substituted by C₁₋₄alkyl and wherein said C₁₋₄alkyl is optionally substituted by NR⁶R⁷, aryl, hydroxy or C₁₋₄alkoxy.

According to a further embodiment of the present invention, R³ is

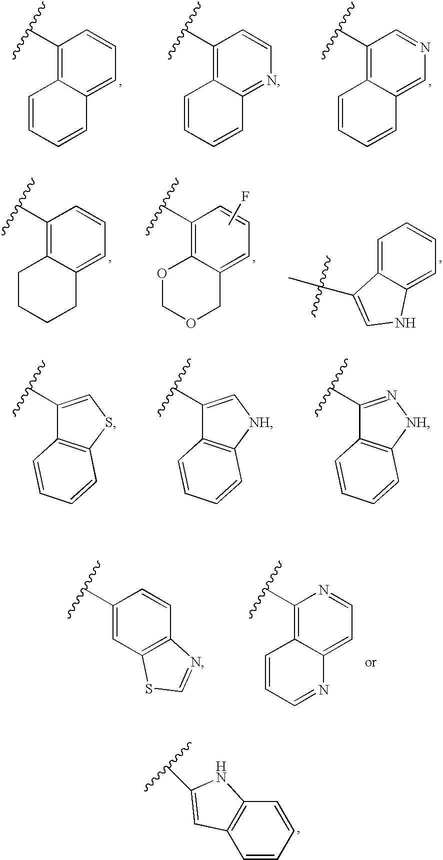

and wherein R³ is optionally substituted by halogen, C₁₋₆alkyl, C₃₋₆cycloalkyl, C₁₋₆alkoxy or C₁₋₆haloalkoxy, wherein said C₁₋₆alkyl or C₃₋₆cycloalkyl is optionally substituted by halogen, NR⁶R⁷, C₁₋₆alkyl, C₃₋₆cycloalkyl, C₁₋₆alkoxy, aryl, heteroaryl or a saturated ring system consisting of 4 to 7 atoms selected from C, N and O, and wherein said C₁₋₆alkyl, C₃₋₆cycloalkyl, aryl, heteroaryl or ring system is optionally substituted by C₁₋₄alkyl and wherein said C₁₋₄alkyl is optionally substituted by NR⁶R⁷, aryl, hydroxy or C₁₋₄alkoxy.

According to yet a further embodiment of the present invention, R³ is selected from

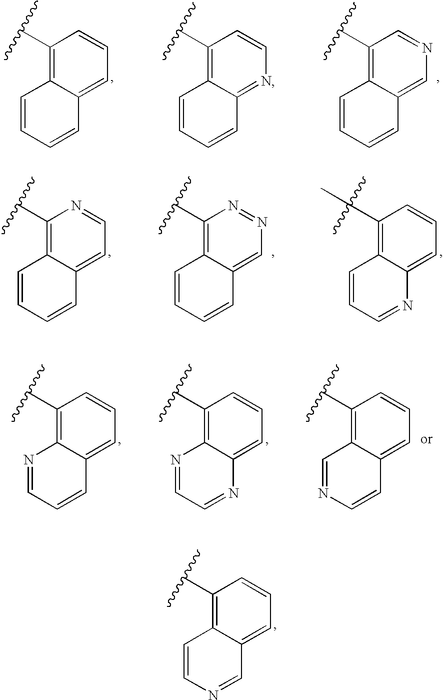

wherein R³ is optionally substituted by halogen, C₁₋₆alkyl, C₃₋₆cycloalkyl, C₁₋₆alkoxy or C₁₋₆haloalkoxy, wherein said C₁₋₆alkyl or C₃₋₆cycloalkyl is optionally substituted by halogen, NR⁶R⁷, C₁₋₆alkyl, C₃₋₆cycloalkyl, C₁₋₆alkoxyaryl or heteroaryl; and wherein said C₁₋₆alkyl, C₃₋₆cycloalkyl, aryl or heteroaryl is optionally substituted by C₁₋₄alkyl and wherein said C₁₋₄alkyl is optionally substituted by NR⁶R⁷, aryl, hydroxy or C₁₋₄alkoxy.

According to another embodiment of the present invention, R³ is naphthyl optionally substituted by halogen, C₁₋₆alkyl, C₃₋₆cycloalkyl, C₁₋₆alkoxy or C₁₋₆haloalkoxy, wherein said C₁₋₆alkyl or C₃₋₆cycloalkyl is optionally substituted by halogen, NR⁶R⁷, C₁₋₆alkyl, C₃₋₆cycloalkyl, C₁₋₆alkoxy, aryl, heteroaryl or a saturated ring system consisting of 4 to 7 atoms selected from C, N and O; and wherein said C₁₋₆alkyl, C₃₋₆cycloalkyl, aryl, heteroaryl or ring system is optionally substituted by C₁₋₄alkyl and wherein said C₁₋₄alkyl is optionally substituted by NR⁶R⁷, aryl, hydroxy or C₁₋₄alkoxy.

According to yet another embodiment of the present invention, R³ is naphthyl substituted by C₁₋₆alkyl, wherein said C₁₋₆alkyl is substituted by heteroaryl; and wherein said heteroaryl is optionally substituted by C₁₋₄alkyl and wherein said C₁₋₄alkyl is optionally substituted by NR⁶R⁷, aryl, hydroxy or C₁₋₄alkoxy. In yet another embodiment of the present invention said C₁₋₆alkyl is methyl. In a further embodiment of the present invention said heteroaryl is 1,2,3-triazolyl.

According to yet another embodiment of the present invention, R³ is selected from:

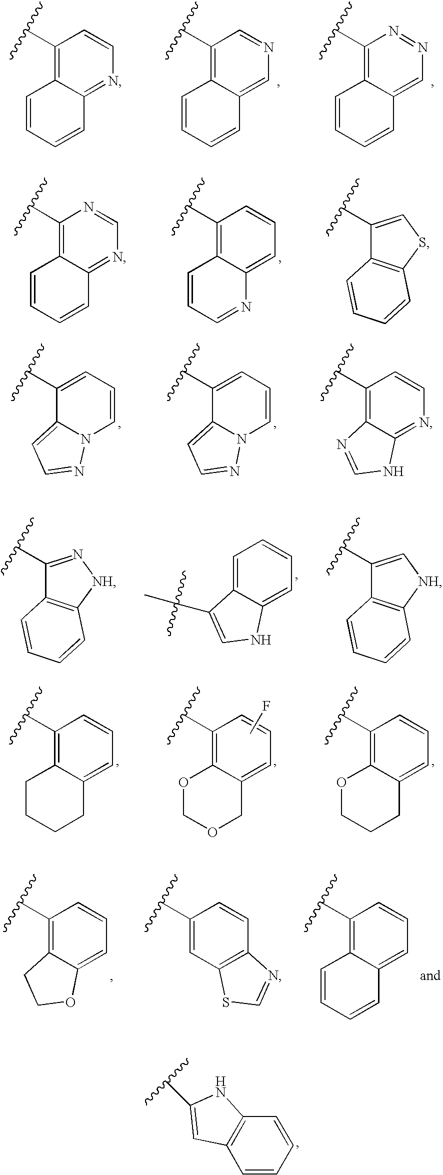

and wherein R³ is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl.

In another embodiment of the present invention R³ is naphthyl substituted by methyl, is wherein said methyl is substituted by 1,2,3-triazolyl; and wherein said 1,2,3-triazolyl is substituted by C₁₋₄alkyl and wherein said C₁₋₄alkyl is optionally substituted by NR⁶R⁷, aryl, hydroxy or C₁₋₄alkoxy.

According to one embodiment of the present invention, R¹ is hydrogen or C₁₋₉alkyl, wherein said C₁₋₉alkyl is optionally substituted by hydroxy, NR^{6a}R^{7a}, C₃₋₆cycloalkyl, aryl or heteroaryl;

R² is hydrogen or C₁₋₆alkyl, wherein said C₁₋₆alkyl is optionally substituted by hydroxy, NR^{6a}R^{7a}, C₃₋₆cycloalkyl, aryl or heteroaryl;
R³ is selected from

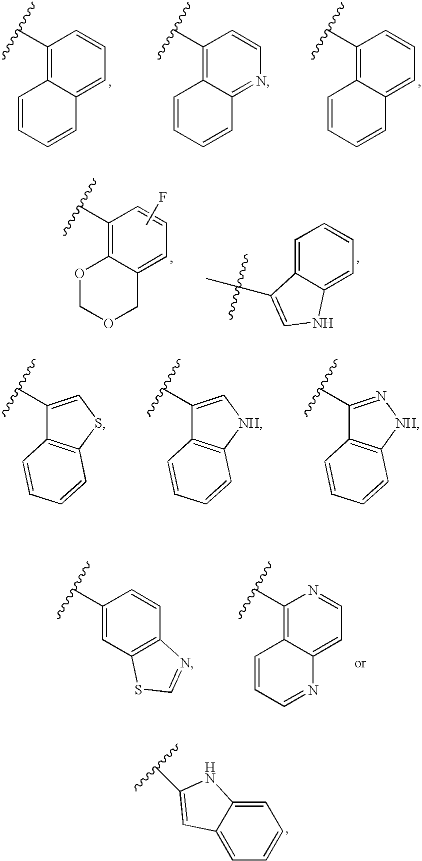

and wherein R³ is substituted by C₁₋₆alkyl, wherein said C₁₋₆alkyl is optionally substituted by heteroaryl or a saturated ring system consisting of 4 to 7 atoms selected from C, N and O, and wherein said heteroaryl or ring system is optionally substituted by C₁₋₄alkyl and wherein said C₁₋₄alkyl is optionally substituted by NR⁶R⁷, aryl, hydroxy or C₁₋₄alkoxy;
R⁴ is hydrogen;
R⁵ is C₃₋₆cycloalkyl;
n is 1;
is R⁶, R^{6a}, R⁷ and R^{7a} are each and independently selected from hydrogen and C₁₋₆alkyl; or R^{6a} and R^{7a} may together form a saturated saturated ring system consisting of 4 to 7 atoms selected from C, O and N; which ring system is optionally substituted with C₁₋₆alkyl, C₁₋₆alkoxy, halogen or hydroxy;
wherein one or more carbon atom(s) of each alkyl or cycloalkyl group as defined for R¹ may be substituted for O, NH, C(O), SO or SO₂ and wherein none of the O or N is in a position adjacent to any other O or N and wherein none of the SO or SO₂ is in a position adjacent to any other SO or SO₂;
wherein one or more carbon atom(s) of each alkyl or cycloalkyl group as defined for R², R³, and R⁵ may be substituted for O, NH, C(O) or SO₂ and wherein none of the O or N is in a position adjacent to any other O or N;
wherein one or more carbon atom(s) of each alkyl or cycloalkyl group as defined for R¹ and R³ may be substituted by fluoro; and
with the proviso that R¹ is not hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl and NR⁶R⁷; at the same time as R² is hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl and NR⁶R⁷; unless R³ is substituted by a C₁₋₄ alkyl, which C₁₋₄alkyl is substituted by a heteroaryl, C₃₋₆cycloalkyl, aryl or a saturated ring system consisting of 4 to 7 atoms selected from C, O and N, wherein said heteroaryl, C₃₋₆cycloalkyl or aryl is further substituted by C₁₋₄alkyl or halogen, wherein said C₁₋₄alkyl is optionally substituted by NR⁶R⁷, aryl, hydroxy or C₁₋₄alkoxy, and wherein said ring system is optionally substituted by C₁₋₄alkyl, wherein said C₁₋₄alkyl is optionally substituted by NR⁶R⁷, aryl, hydroxy or C₁₋₄alkoxy;
or unless R³ is selected from:

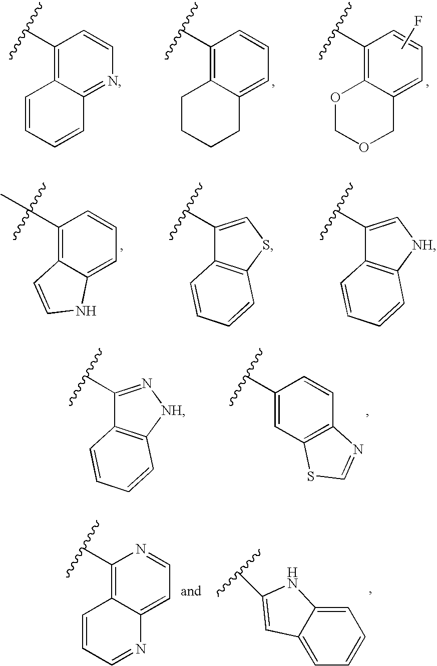

and wherein R³ is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
or a pharmaceutically acceptable salt thereof, or diastereomers, or enantiomers, or mixtures thereof. According to a further embodiment of the present invention, A¹ is N and A² is N. According to yet a further embodiment of the present invention, A¹ is N and A² is CH.

In another embodiment of the present invention, R¹ is selected from hydrogen or C₁₋₆alkyl, said C₁₋₆alkyl is optionally substituted by hydroxy, NR^{6a}R^{7a}, C₃₋₆cycloalkyl, aryl or heteroaryl;

R² is hydrogen;
R³ is naphthyl, optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl, wherein said C₁₋₆alkyl, C₃₋₆cycloalkyl, aryl or heteroaryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl; and wherein said C₁₋₆alkyl, C₃₋₆cycloalkyl, aryl or heteroaryl is optionally further substituted by C₁₋₄alkyl and wherein said C₁₋₄alkyl is optionally substituted by NR⁶R⁷, aryl, hydroxy or C₁₋₄alkoxy;
R⁴ is hydrogen;
R⁵ is C₃₋₆cycloalkyl;
R⁶ is hydrogen or C₁₋₄alkyl;
R⁷ is hydrogen or C₁₋₄alkyl;
R^{6a} is hydrogen or C₁₋₄alkyl; and
R^{7a} is hydrogen or C₁₋₄alkyl.

The present invention also relates to a compound selected from:
Methyl[(6-{[(cyclohexylmethyl)amino]carbonyl}-5-{[4-(1H-1,2,3-triazol-1-ylmethyl)-1-naphthoyl]amino}pyridin-2-yl)oxy]acetate;
Methyl[(6-{[(cyclobutylmethyl)amino]carbonyl}-5-{[4-(1H-1,2,3-triazol-1-ylmethyl)-1-naphthoyl]amino}pyridin-2-yl)oxy]acetate;
[(6-{[(Cyclobutylmethyl)amino]carbonyl}-5-{[4-(1H-1,2,3-triazol-1-ylmethyl)-1-naphthoyl]amino}pyridin-2-yl)oxy]acetic acid;
6-(2-Amino-2-oxoethoxy)-N-(cyclobutylmethyl)-3-{[4-(1H-1,2,3-triazol-1-ylmethyl)-1-naphthoyl]amino}pyridine-2-carboxamide;
N-(cyclobutylmethyl)-6-[2-(methylamino)-2-oxoethoxy]-3-{[4-(1H-1,2,3-triazol-1-ylmethyl)-1-naphthoyl]amino}pyridine-2-carboxamide;
N-(cyclobutylmethyl)-6-[2-(dimethylamino)-2-oxoethoxy]-3-{[4-(1H-1,2,3-triazol-1-ylmethyl)-1-naphthoyl]amino}pyridine-2-carboxamide;
N-(cyclobutylmethyl)-6-{2-[(2-hydroxyethyl)amino]-2-oxoethoxy}-3-{[4-(1H-1,2,3-triazol-1-ylmethyl)-1-naphthoyl]-amino}pyridine-2-carboxamide;
6-{[(Cyclobutylmethyl)amino]carbonyl}-5-{[4-(1H-1,2,3-triazol-1-ylmethyl)-1-naphthoyl]amino}pyridin-2-yl ethanesulfonate;
6-{[(Cyclobutylmethyl)amino]carbonyl}-5-{[4-(1H-1,2,3-triazol-1-ylmethyl)-1-naphthoyl]amino}pyridin-2-yl 3,3,3-trifluoropropane-1-sulfonate;
6-{[(Tetrahydro-2H-pyran-4-ylmethyl)amino]carbonyl}-5-{[4-(16H-1,2,3-triazol-1-ylmethyl)-1-naphthoyl]amino}pyridin-2-yl 3,3,3-trifluoropropane-1-sulfonate;
6-{[(Tetrahydro-2H-pyran-4-ylmethyl)amino]carbonyl}-5-{[4-(1H-1,2,3-triazol-1-ylmethyl)-1-naphthoyl]amino}pyridin-2-yl acetate;
N-(cyclobutylmethyl)-6-(2-hydroxyethoxy)-3-{[4-(1H-1,2,3-triazol-1-ylmethyl)-1-naphthoyl]amino}pyridine-2-carboxamide;
N-(cyclobutylmethyl)-6-[2-(2-hydroxyethoxy)ethoxy]-3-{[4-(1H-1,2,3-triazol-1-ylmethyl)-1-naphthoyl]amino}pyridine-2-carboxamide;
6-(Benzyloxy)-N-(tetrahydro-2H-pyran-4-ylmethyl)-3-{[4-(1H-1,2,3-triazol-1-ylmethyl)-1-naphthoyl]amino}pyridine-2-carboxamide;
3-Benzyl-1-[(4-{[(6-(benzyloxy)-2-{[(tetrahydro-2H-pyran-4-ylmethyl)amino]carbonyl}pyridin-3-yl)amino]carbonyl}-1-naphthyl)methyl]-1H-1,2,3-triazol-3-ium;
N-(cyclobutylmethyl)-6-(pyridin-2-ylmethoxy)-3-{[4-(1H-1,2,3-triazol-1-ylmethyl)-1-naphthoyl]amino}pyridine-2-carboxamide;
N-(cyclobutylmethyl)-3-[(4-{[5-(methoxymethyl)-1H-1,2,3-triazol-1-yl]methyl}-1-naphthoyl)amino]pyridine-2-carboxamide;
N-(cyclobutylmethyl)-3-[(4-{[4-(methoxymethyl)-1H-1,2,3-triazol-1-yl]methyl}-1-naphthoyl)amino]pyridine-2-carboxamide;
N-(cyclobutylmethyl)-3-[(4-{[5-(1-hydroxyethyl)-1H-1,2,3-triazol-1-yl]methyl}-1-naphthoyl)amino]pyridine-2-carboxamide;
N-(cyclobutylmethyl)-3-[(4-{[4-(1-hydroxyethyl)-1H-1,2,3-triazol-1-yl]methyl}-1-naphthoyl)amino]pyridine-2-carboxamide;
3-[(4-{[5-(aminocarbonyl)-1H-1,2,3-triazol-1-yl]methyl}-1-naphthoyl)amino]-N-(cyclobutylmethyl)pyridine-2-carboxamide;
3-[(4-{[4-(aminocarbonyl)-1H-1,2,3-triazol-1-yl]methyl}-1-naphthoyl)amino]-N-(cyclobutylmethyl)pyridine-2-carboxamide;
6-(Aminomethyl)-N-(cyclobutylmethyl)-3-{[4-(1H-1,2,3-triazol-1-ylmethyl)-1-naphthoyl]amino}pyridine-2-carboxamide;
N-(cyclobutylmethyl)-6-(hydroxymethyl)-3-{[4-(1H-1,2,3-triazol-1-ylmethyl)-1-naphthoyl]amino}pyridine-2-carboxamide;
N-(cyclobutylmethyl)-6-{[(methylsulfonyl)amino]methyl}-3-{[4-(1H-1,2,3-triazol-1-ylmethyl)-1-naphthoyl]amino}pyridine-2-carboxamide;
Methyl 6-{[(cyclobutylmethyl)amino]carbonyl}-5-{[4-(1H-1,2,3-triazol-1-yl methyl)-1-naphthoyl]amino}pyridine-2-carboxylate;
N²-(cyclobutylmethyl)-3-{[4-(1H-1,2,3-triazol-1-ylmethyl)-1-naphthoyl]amino}pyridine-2,6-dicarboxamide;
and N-(cyclobutylmethyl)-6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxamide.

The present invention also relates to a compound selected from:
6-(2-Morpholin-4-yl-2-oxo-ethoxy)-3-[(4-[1,2,3]triazol-1-ylmethyl-naphthalene-1-carbonyl)-amino]-pyridine-2-carboxylic acid cyclobutylmethyl-amide;
6-(Benzylcarbamoyl-methoxy)-3-[(4-[1,2,3]triazol-1-ylmethyl-naphthalene-1-carbonyl)-amino]-pyridine-2-carboxylic acid cyclobutylmethyl-amide;
{6-(Cyclobutylmethyl-carbamoyl)-5-[(4-[1,2,3]triazol-1-ylmethyl-naphthalene-1-carbonyl)-amino]-pyridin-2-yloxy}-acetic acid 2,2-dimethyl-propyl ester;
{6-(Cyclobutylmethyl-carbamoyl)-5-[(4-[1,2,3]triazol-1-ylmethyl-naphthalene-1-carbonyl)-amino]-pyridin-2-yloxy}-acetic acid isopropyl ester;
6-Hydroxycarbamoylmethoxy-3-[(4-[1,2,3]triazol-1-ylmethyl-naphthalene-1-carbonyl)-amino]-pyridine-2-carboxylic acid cyclobutylmethyl-amide;
6-(Methoxycarbamoyl-methoxy)-3-[(4-[1,2,3]triazol-1-ylmethyl-naphthalene-1-carbonyl)-amino]-pyridine-2-carboxylic acid cyclobutylmethyl-amide;
{5-[(4-Methyl-naphthalene-1-carbonyl)-amino]-6-[(tetrahydro-pyran-4-ylmethyl)-carbamoyl]-pyridin-2-yloxy}-acetic acid methyl ester;
6-Carbamoylmethoxy-3-[(4-methyl-naphthalene-1-carbonyl)-amino]-pyridine-2-carboxylic acid (tetrahydro-pyran-4-ylmethyl)-amide;
{5-[(4-Methyl-naphthalene-1-carbonyl)-amino]-6-[(tetrahydro-pyran-4-ylmethyl)-carbamoyl]-pyridin-2-yloxy}-acetic acid;
6-(2-Hydroxy-ethoxy)-3-[(4-methyl-naphthalene-1-carbonyl)-amino]-pyridine-2-carboxylic acid (tetrahydro-pyran-4-ylmethyl)-amide;
6-(2-Hydroxy-ethoxy)-3-[(4-methoxymethyl-naphthalene-1-carbonyl)-amino]-pyridine-2-carboxylic acid (tetrahydro-pyran-4-ylmethyl)-amide;
6-Methanesulfonyl-3-[(4-methyl-naphthalene-1-carbonyl)-amino]-pyridine-2-carboxylic acid (tetrahydro-pyran-4-ylmethyl)-amide;
6-Methanesulfinyl-3-[(4-methyl-naphthalene-1-carbonyl)-amino]-pyridine-2-carboxylic acid (tetrahydro-pyran-4-ylmethyl)-amide;
6-[2-(2-Hydroxy-ethoxy)-ethoxy]-3-[(4-[1,2,3]triazol-1-ylmethyl-naphthalene-1-carbonyl)-amino]-pyridine-2-carboxylic acid (tetrahydro-pyran-4-ylmethyl)-amide;
6-methoxy-3-({4-[(4-methylpiperazin-1-yl)methyl]-1-naphthoyl}amino)-N-(tetrahydro-2H-pyran-4-ylmethyl)pyridine-2-carboxamide;
6-methoxy-3-{[4-(morpholin-4-ylmethyl)-1-naphthoyl]amino}-N-(tetrahydro-2H-pyran-4-ylmethyl)pyridine-2-carboxamide;
6-[(ethylamino)sulfonyl]-3-{[4-(methoxymethyl)-1-naphthoyl]amino}-N-(tetrahydro-2H-pyran-4-ylmethyl)pyridine-2-carboxamide;
6-(benzylsulfonyl)-3-{[4-(methoxymethyl)-1-naphthoyl]amino}-N-(tetrahydro-2H-pyran-4-ylmethyl)pyridine-2-carboxamide;
6-(benzylsulfonyl)-3-{[4-(methoxymethyl)-1-naphthoyl]amino}-N-(tetrahydro-2H-pyran-4-ylmethyl)pyridine-2-carboxamide;
6-[(Tetrahydro-2H-pyran-4-ylmethyl)carbamoyl]-5-{[4-(1H-1,2,3-thiazol-1-ylmethyl)-1-naphthoyl]amino}pyrazin-2-yl 3,3,3-trifluoropropane-1-sulfonate;
N-(Cyclobutylmethyl)-3-{[4-({5-[(dimethylamino)methyl]-1H-1,2,3-triazol-1-yl}methyl)-1-naphthoyl]amino}pyridine-2-carboxamide;
N-(Cyclobutylmethyl)-3-{[4-({4-[(dimethylamino)methyl]-1H-1,2,3-triazol-1-yl}methyl)-1-naphthoyl]amino}pyridine-2-carboxamide;
N-(Cyclobutylmethyl)-3-[(4-{[4-(trifluoromethyl)-1H-1,2,3-triazol-1-yl]methyl}-1-naphthoyl)amino]pyridine-2-carboxamide;
N-(Cyclobutylmethyl)-3-[(4-{[5-(phenylsulfonyl)-1H-1,2,3-triazol-1-yl]methyl}-1-naphthoyl)amino]pyridine-2-carboxamide;
N-(cyclobutylmethyl)-3-[(4-{[4-(phenylsulfonyl)-1H-1,2,3-triazol-1-yl]methyl}-1-naphthoyl)amino]pyridine-2-carboxamide;
N-(Cyclobutylmethyl)-3-({4-[(4-fluoro-1H-1,2,3-triazol-1-yl)methyl]-1-naphthoyl}amino)pyridine-2-carboxamide;
N-{2-[(cyclobutylmethyl)carbamoyl]pyridin-3-yl}-1-methyl-1H-indole-3-carboxamide;
N-{2-[(cyclobutylmethyl)carbamoyl]pyridin-3-yl}-1-methyl-1H-indole-2-carboxamide;
N-{2-[(cyclobutylmethyl)carbamoyl]pyridin-3-yl}-1H-indole-3-carboxamide;
N-{2-[(cyclobutylmethyl)carbamoyl]-4-methoxyphenyl}quinoline-4-carboxamide;
N-{2-[(cyclobutylmethyl)carbamoyl]-6-methoxypyridin-3-yl}-1-methyl-1H-indazole-3-carboxamide;
3-[(1-benzothien-3-yl-carbonyl)amino]-N-(tetrahydro-2H-pyran-4-ylmethyl)pyridine-2-carboxamide;
3-[(5,6,7,8-tetrahydronaphthalen-1-ylcarbonyl)amino]-N-(tetrahydro-2H-pyran-4-ylmethyl)pyridine-2-carboxamide;
N-{2-[(tetrahydro-2H-pyran-4-ylmethyl)carbamoyl]pyridin-3-yl}-1H-indazole-3-carboxamide;
N-{2-[(tetrahydro-2H-pyran-4-ylmethyl)carbamoyl]pyridin-3-yl}-1H-indole-3-carboxamide;
1-methyl-N-{2-[(tetrahydro-2H-pyran-4-ylmethyl)carbamoyl]pyridin-3-yl}-1H-indole-3-carboxamide;
N-{2-[(tetrahydro-2H-pyran-4-ylmethyl)carbamoyl]pyridin-3-yl}-1,3-benzothiazole-6-carboxamide;
N-{2-[(tetrahydro-2H-pyran-4-ylmethyl)carbamoyl]pyridin-3-yl}-1,6-naphthyridine-5-carboxamide;
3-{[(6-fluoro-4H-1,3-benzodioxin-8-yl)carbonyl]amino}-N-(tetrahydro-2H-pyran-4-ylmethyl)pyridine-2-carboxamide;
N-{2-[(cyclobutylmethyl)carbamoyl]pyridin-3-yl}-1H-indazole-3-carboxamide; and
3-[(4-{[(5-methylisoxazol-3-yl)methoxy]methyl}-1-naphthoyl)amino]-N-(tetrahydro-2H-pyran-4-ylmethyl)pyridine-2-carboxamide.

The present invention also relates to a compound selected from:
N-(cyclobutylmethyl)-6-hydroxy-3-{[4-(1H-1,2,3-triazol-1-ylmethyl)-1-naphthoyl]amino}pyridine-2-carboxamide;
6-methoxy-N-(tetrahydro-2H-pyran-4-ylmethyl)-3-{[4-(1H-1,2,3-triazol-1-ylmethyl)-1-naphthoyl]amino}pyridine-2-carboxamide;
methyl 3-[(4-{[5-(methoxymethyl)-1H-1,2,3-triazol-1-yl]methyl}-1-naphthoyl)amino]pyridine-2-carboxylate;
methyl 3-[(4-{[4-(methoxymethyl)-1H-1,2,3-triazol-1-yl]methyl}-1-naphthoyl)amino]pyridine-2-carboxylate;
methyl 3-{[4-(azidomethyl)-1-naphthoyl]amino}pyridine-2-carboxylate;
6-Cyano-N-(cyclobutylmethyl)-3-{[4-(1H-1,2,3-triazol-1-ylmethyl)-1-naphthoyl]amino}pyridine-2-carboxamide;
methyl 6-cyano-3-[(4-methyl-1-naphthoyl)amino]pyridine-2-carboxylate;
methyl 6-chloro-3-[(4-methyl-1-naphthoyl)amino]pyridine-2-carboxylate;
6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxylic acid; methyl 6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxylate;
methyl 5-chloro-6-methoxy-3-{[4-(1H-1,2,3-triazol-1-ylmethyl)-1-naphthoyl]amino}pyrazine-2-carboxylate;
methyl-5-chloro-6-methoxy-3-[(4-methyl-1-naphthoyl)amino]pyrazine-2-carboxylate;
methyl-6-methoxy-3-[(4-methyl-1-naphthoyl)amino]pyrazine-2-carboxylate;
methyl-3-amino-6-methoxypyrazine-2-carboxylate;
3-Amino-6-methoxypyrazine-2-carboxylic acid;
6-Bromo-3-(3-chlorophenyl)pteridine-2,4(1H,3H)-dione;
methyl-3-{[4-(bromomethyl)-1-naphthoyl]amino}pyridine-2-carboxylate;
methyl-3-amino-6-bromopyrazine-2-carboxylate;
6-Hydroxy-3-[(4-methyl-naphthalene-1-carbonyl)-amino]-pyridine-2-carboxylic acid (tetrahydro-pyran-4-ylmethyl)-amide;
6-Hydroxy-N-(tetrahydro-2H-pyran-4-ylmethyl)-3-{[4-(1H-1,2,3-triazol-1-ylmethyl)-1-naphthoyl]amino}pyrazine-2-carboxamide;
3-[(4-Methyl-naphthalene-1-carbonyl)-amino]-6-methylsulfanyl-pyridine-2-carboxylic acid (tetrahydro-pyran-4-ylmethyl)-amide;
6-Chloro-3-[(4-methyl-naphthalene-1-carbonyl)-amino]-pyridine-2-carboxylic acid (tetrahydro-pyran-4-ylmethyl)-amide;
6-chloro-3-{[4-(methoxymethyl)-1-naphthoyl]amino}-N-(tetrahydro-2H-pyran-4-ylmethyl)pyridine-2-carboxamide;
3-{[4-(bromomethyl)-1-naphthoyl]amino}-6-methoxy-N-(tetrahydro-2H-pyran-4-ylmethyl)pyridine-2-carboxamide;
6-(benzylthio)-3-{[4-(methoxymethyl)-1-naphthoyl]amino}-N-(tetrahydro-2H-pyran-4-ylmethyl)pyridine-2-carboxamide;
Methyl-5-methoxy-2-[(quinolin-4-ylcarbonyl)amino]benzoate; and
Methyl-6-methoxy-3-{[(1-methyl-1H-indazol-3-yl)carbonyl]amino}pyridine-2-carboxylate. The present invention also relates to the use the above-mentioned compounds in a process for manufacture a compound according to the present invention.

The present invention also relates to a pharmaceutical composition comprising a compound of formula (I) as defined above as an active ingredient and a pharmaceutically acceptable carrier or diluent.

The compounds of the present invention have activity as pharmaceuticals, in particular as modulators or ligands such as agonists, partial agonists, inverse agonist or antagonists of CB₁ receptors. More particularly, the compounds of the present invention exhibit activity as agonist of the CB₁ receptors and are useful in therapy, especially for relief of various gastrointestinal disorders, e.g. gastroesophageal reflux disease, constipation, functional gastrointestinal disorders such as Irritable Bowel Syndrome (IBS) and Functional Dyspepsia (FD). The compounds of the present invention are also useful for the relief of various pain conditions such as chronic pain, neuropathic pain, acute pain, cancer pain, back pain, pain caused by rheumatoid arthritis, migraine, visceral pain etc. These lists should however not be interpreted as exhaustive. Additionally, compounds of the present invention are useful in other disease states in which dysfunction of CB₁ receptors is present or implicated. Furthermore, the compounds of the present invention may be used to treat cancer, multiple sclerosis, Parkinson's disease, Huntington's chorea, Alzheimer's disease, anxiety disorders, and cardiovascular disorders.

The compounds of the present invention are useful as immunomodulators, especially for autoimmune diseases, such as arthritis, for skin grafts, organ transplants and similar surgical needs, for collagen diseases, various allergies, for use as anti-tumour agents and anti viral agents.

The compounds of the present invention are useful in disease states where degeneration or dysfunction of cannabinoid receptors is present or implicated in that paradigm. This may involve the use of isotopically labeled versions of the compounds of the invention in diagnostic techniques and imaging applications such as positron emission tomography (PET).

The compounds of the present invention are also useful for the treatment of diarrhea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, Parkinson's disease and other motor disorders, traumatic brain injury, stroke, cardioprotection following myocardial infarction, spinal injury and drug addiction, including the treatment of alcohol, nicotine, opioid and other drug abuse and for disorders of the sympathetic nervous system for example hypertension.

The compounds of the present invention are useful as an analgesic agent for use during general anaesthesia and monitored anaesthesia care. Combinations of agents with different is properties are often used to achieve a balance of effects needed to maintain the anaesthetic state (e.g. amnesia, analgesia, muscle relaxation and sedation). Included in this combination are inhaled anesthetics, hypnotics, anxiolytics, neuromuscular blockers and opioids.

Another aspect of the present invention is the use of a compound according to formula (I), for the inhibition of transient lower esophageal sphincter relaxations (TLESRs) and thus for treatment or prevention of gastroesophageal reflux disorder (GERD). The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. In yet further embodiments of the present invention, the compound according to formula (I) are useful for the prevention of reflux, treatment or prevention of regurgitation, treatment or prevention of asthma, treatment or prevention of laryngitis, treatment or prevention of lung disease and for the management of failure to thrive.

A further aspect of the present invention is the use of a compound according to formula (I), for the manufacture of a medicament for the inhibition of transient lower esophageal sphincter relaxations, for the treatment or prevention of GERD, for the prevention of reflux, for the treatment or prevention of regurgitation, treatment or prevention of asthma, treatment or prevention of laryngitis, treatment or prevention of lung disease and for the management of failure to thrive.

Still another aspect of the present invention is the use of a compound according to formula (I) for the manufacture of a medicament for the treatment or prevention of functional gastrointestinal disorders, such as functional dyspepsia (FD). Yet another aspect of the present invention is the use of a compound according to formula (I) for the manufacture of a medicament for the treatment or prevention of irritable bowel syndrome (IBS), such as constipation predominant IBS, diarrhea predominant IBS or alternating bowel movement predominant IBS. Exemplary irritable bowel syndrome (IBS) and functional gastrointestinal disorders (FGD), such as functional dyspepsia (FD), are illustrated in Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders Diagnosis, Pathophysiology and Treatment. 2 ed. McLean, V A: Degnon Associates, Inc.; 2000:351-432 and Drossman D A, Corazziari E, Talley N J, Thompson W G and Whitehead W E. Rome II: A multinational consensus document on Functional Gastrointestinal Disorders. Gut 45(Suppl. 2), II1-II81.9-1-1999.

Also within the scope of the present invention is the use of any of the compounds according to the formula (I) above, for the manufacture of a medicament for the treatment of any of the conditions discussed above.

A further aspect of the present invention is a method for the treatment of a subject suffering from any of the conditions discussed above, whereby an effective amount of a compound according to the formula (I) above, is administered to a patient in need of such treatment.

Thus, the invention provides a compound of formula (I), or pharmaceutically acceptable salt or solvate thereof, as hereinbefore defined for use in therapy.

In a further aspect, the present invention provides the use of a compound of formula (I), or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders. The is compounds of the present invention are useful in therapy, especially for the therapy of various pain conditions including, but not limited to: acute pain, chronic pain, neuropathic pain, back pain, cancer pain, and visceral pain.

In use for therapy in a warm-blooded animal such as a human, the compound of the present invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

A further aspect of the present invention is a method for treatment of a subject suffering from any of the conditions discussed above, whereby an effective amount of a compound according to the formula (I) above, is administered to a patient in need of such treatment.

### Pharmaceutical Formulations

In one embodiment of the present invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this present invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavouring agents, solubilizers, lubricants, suspending agents, binders, or table disintegrating agents; it can also be an encapsulating material.

In powders, the carrier is a finely divided solid, which is in a mixture with the finely divided compound of the present invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavouring agents, stabilizers, and thickening agents as desired. Aqueous suspensions for oral use can be made by dispersing the finely divided active component in water together with a viscous material such as natural synthetic gums, resins, methylcellulose, sodium carboxymethyl cellulose, and other suspending agents known to the pharmaceutical formulation art.

Depending on the mode of administration, the pharmaceutical composition will according to one embodiment of the present invention include from 0.05% to 99% w (percent by weight), according to an alternative embodiment from 0.10 to 50% w, of the compound of the present invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art. A typical daily dose of the cannabinoid receptor agonist is 0.1-10 mg, but this will depend on various factors such as the route of administration, the age and weight of the patient as well as of severity of the patient's condition.

Additionally, there is provided a pharmaceutical composition comprising a compound of formula (I), or a pharmaceutically acceptable salt thereof, in association with a pharmaceutically acceptable carrier.

The present invention also relates to a pharmaceutical composition comprising a compound of formula (I), or a pharmaceutically acceptable salt thereof, in association with a pharmaceutically acceptable carrier or diluent for therapy.

Further, there is provided a pharmaceutical composition comprising a compound of formula (I), or a pharmaceutically acceptable salt thereof, in association with a pharmaceutically acceptable carrier use in any of the conditions discussed above.

### Methods of Preparation

The present invention provides a method for preparing a compound of formula (I),

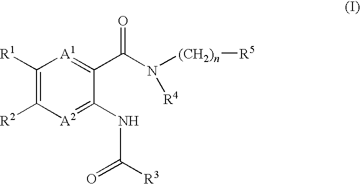

wherein R¹, R², R³, R⁴, R⁵, A¹, A² and n are as defined above,
comprising:
(i) reacting a compound of formula (II)

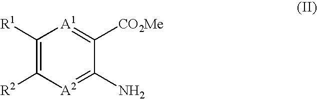

with R³COCl in a solvent, such as CH₂Cl₂, in the presence of a base, such as an DIPEA, whereby a compound of formula (III) is obtained (wherein R¹, R², R³, A¹ and A² are as defined above);
(ii) reacting the compound of formula (III) obtained from step (i),

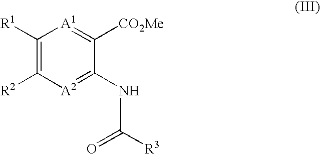

with R⁴(CH₂)ₙR⁵NH, in a solvent, such as DMF (wherein R¹, R², R³, R⁴, R⁵, n, A¹ and A² are as defined above).

Compounds of the present invention may also be prepared according to the synthetic routes as depicted in Schemes 1-4. In Schemes 1-4, R¹, R², A¹, A², R³, R⁴, R⁵, n, R⁶ and R⁷ are as defined above unless specified otherwise.

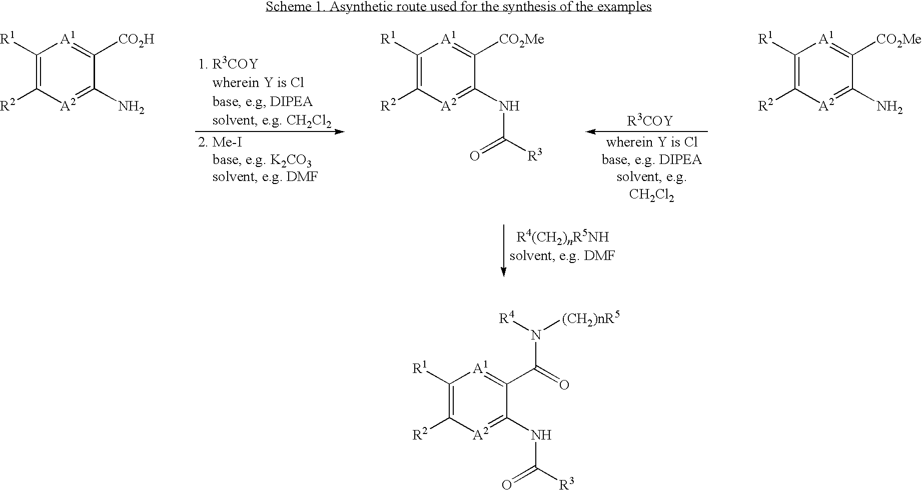

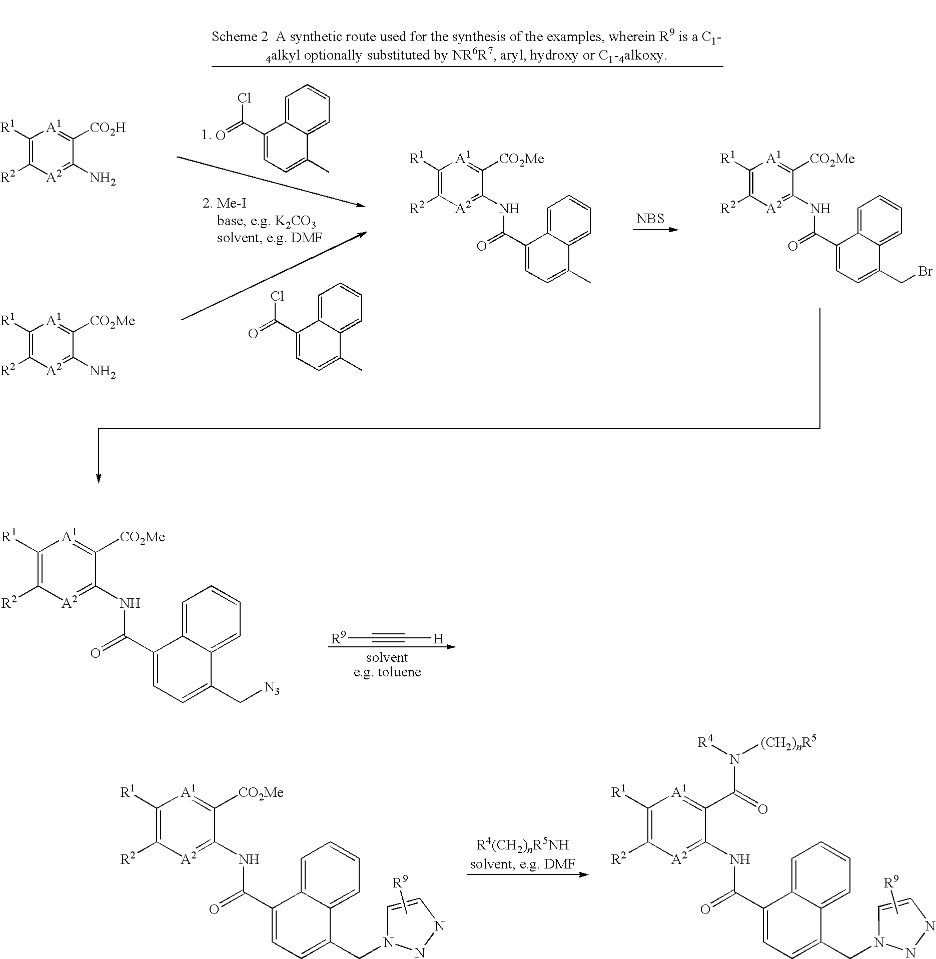

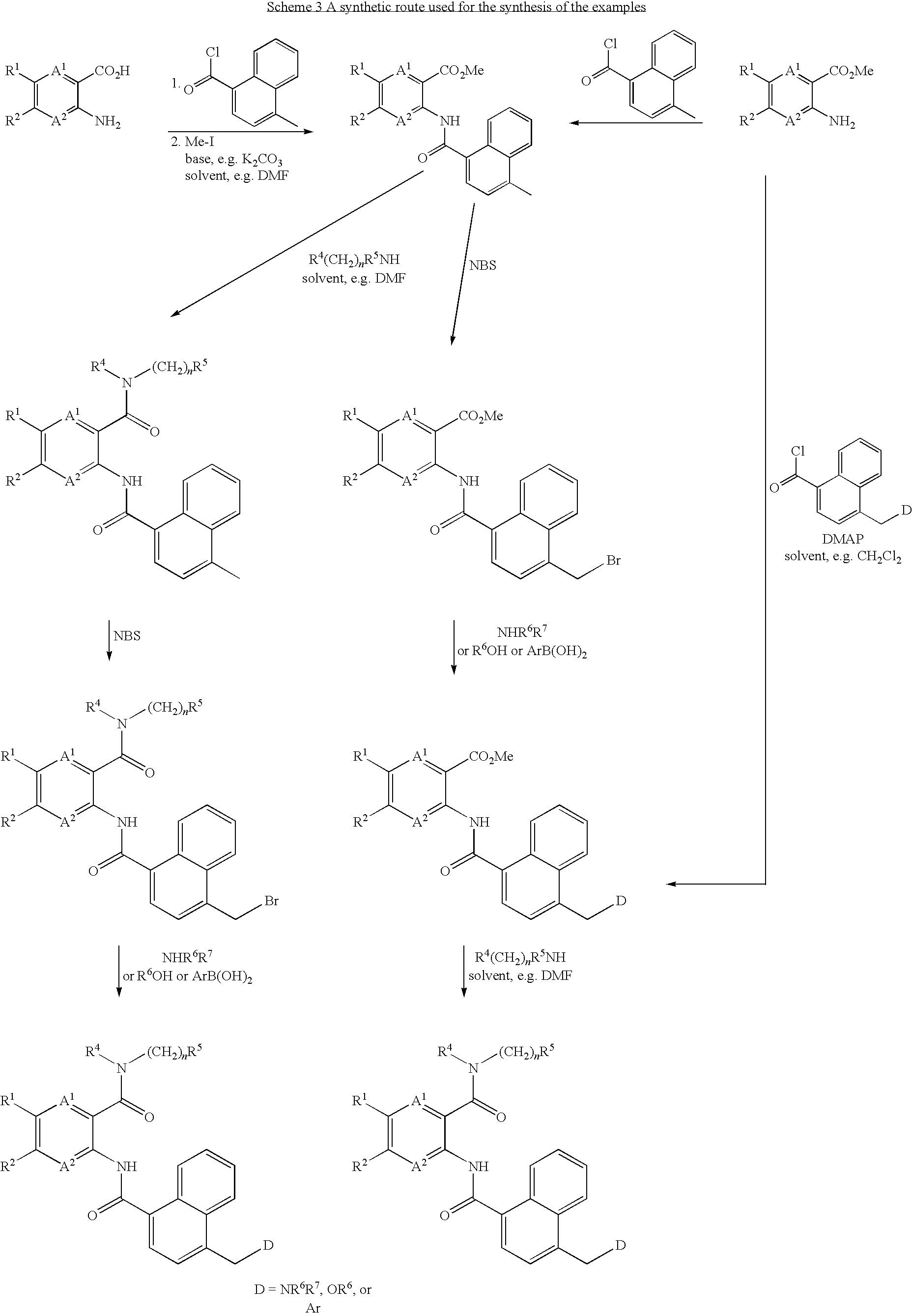

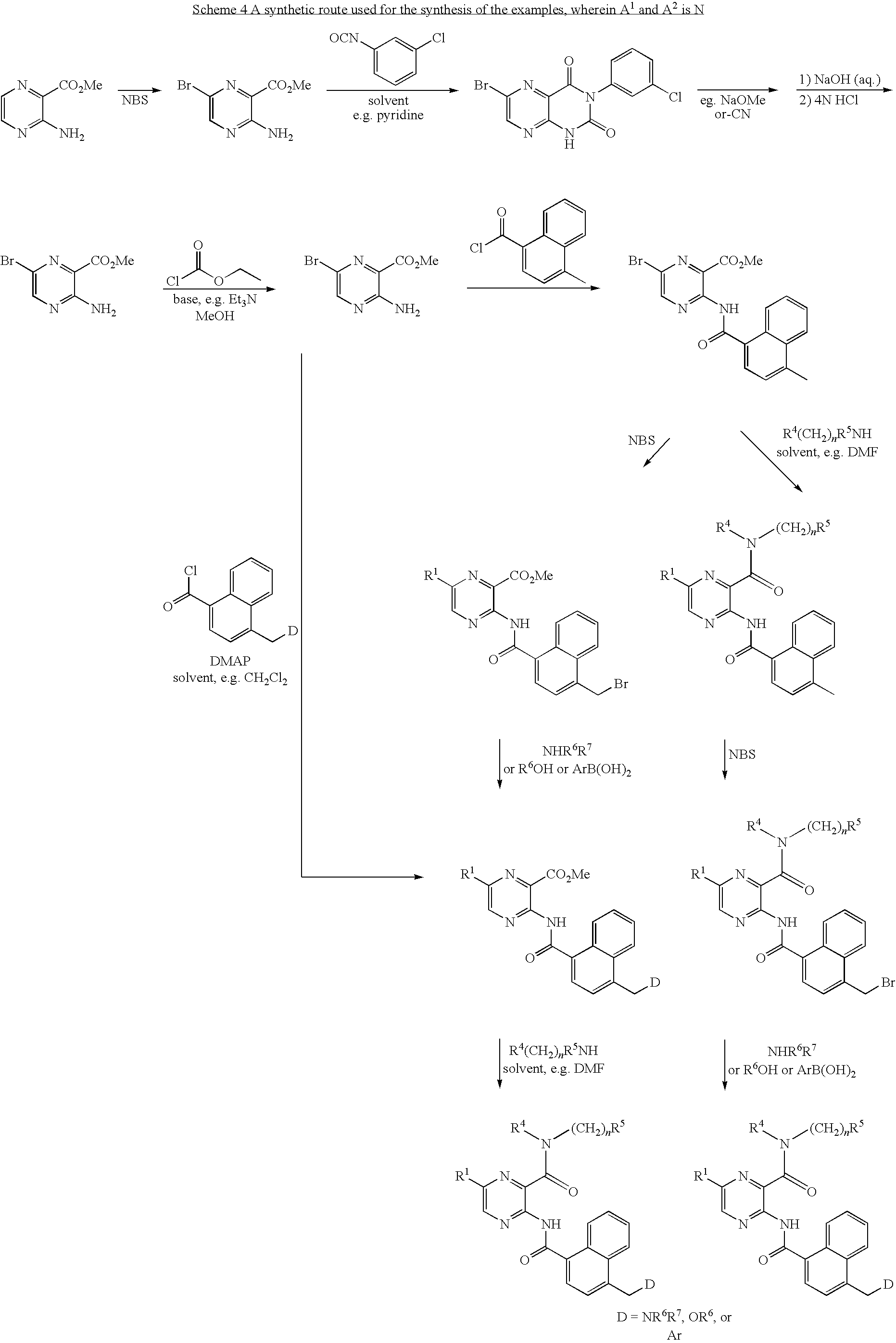

### General Procedures

### General Procedure 1 (Wherein R¹ and R² are as Defined Above):

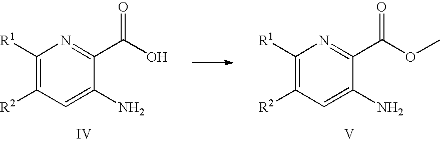

The compound of general formula (IV) was dissolved in MeOH (10 mL/mmol) and treated with TMSCl (10 equiv.) or HCl (g) for 48 h at room temperature. The solvent was removed under reduced pressure and the residue was dissolved in CH₂Cl₂ and washed with sat NaHCO₃ (aq) to afford the compound with the general formula (V).

### General Procedure 2 (Wherein R³ is as Defined Above):

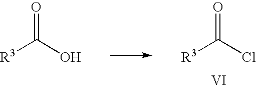

The compound of general formula (VI) was prepared from the corresponding carboxylic acid by treatment with oxalyl chloride (1.3→3 equiv.) in CH₂Cl₂ (8 mL/mmol) at room temperature for 2→16 h. The solvent was removed under reduced pressure to afford compound of general formula (II).

### General Procedure 3 (Wherein R¹, R² and R³ are as Defined Above):

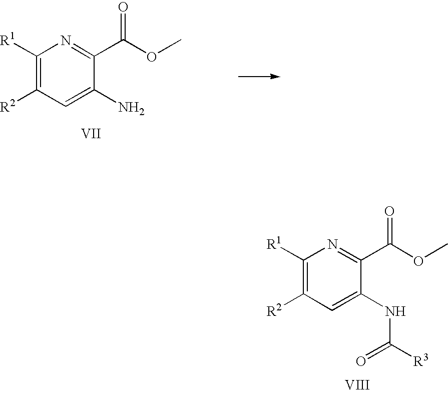

The compound of general formula (VII) was dissolved in CHCl₃ (2.5 mL/mmol) and treated with pyridine (5→10 equiv.) and 4-dimethylaminopyridine (DMAP, 0.3 equiv.). The reaction mixture was heated to 50→60° C. and treated with a compound of the general formula (VI) (1.5 equiv.) in CHCl₃ (1.7 mL/mmol and pyridine 0→10 equiv.). The reaction was run at 50→60° C. for 1→2 h to afford compounds of general formula (VIII).

### General Procedure 4 (Wherein R¹, R², R³ and R⁴ are as Defined Above):

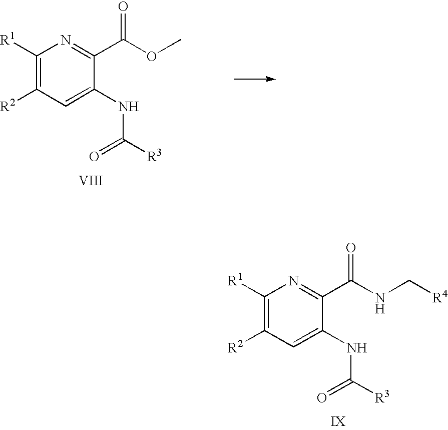

Compound of general formula (VIII) was dissolved in DMF (10 mL/mmol) and treated with amines of general formula H₂NCH₂R⁴ (3→6 equiv.) at 80→100° C. for 3→6 h to afford compound of general formula (IX).

### General Procedure 5 (Wherein R⁴ is as Defined Above and R¹ and R² are Hydrogen):

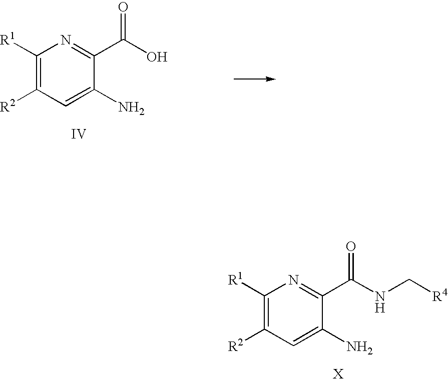

Compound of general formula (IV) was dissolved in CH₂Cl₂ (4 mL/mmol) and treated with DIPEA (3→5 equiv.), amine of general formula H₂NCH₂R⁴ (1.2 equiv.) and TBTU (1.3 equiv.) at room temperature for 1→2 h to afford compounds of general formula (X).

### General Procedure 6 (Wherein R¹, R², R³ and R⁴ are as Defined Above):

The compound of general formula (X) was dissolved in CH₂Cl₂ (5 mL/mmol) and DIPEA (10 equiv) and added to a compound of the general formula (VI) (3 equiv.) dissolved in CH₂Cl₂ (5 mL/mmol at ambient temperature. The reaction was run at ambient temperature over night (16 h) to afford compounds of general formula (V).

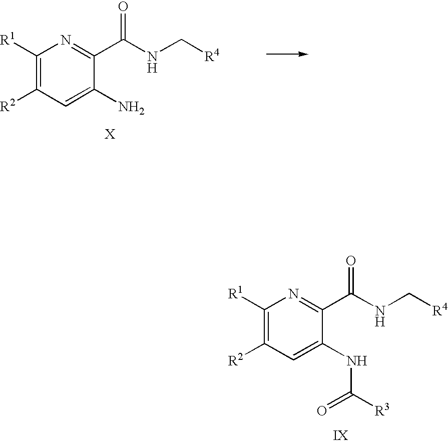

### General Procedure 6b (Wherein R¹, R², R³ and R⁴ are as Defined Above):

The compound of general formula (X) was dissolved in DMF (6 mL/mmol) and treated with DIPEA (2 equiv.), acid of general formula (IV) (1 equiv.) and TBTU (1 equiv.) at room temperature for 1→2 h to afford compounds of general formula (IX).

### Biological Evaluation

hCB₁ and hCB₂ Receptor Binding

Membranes are produced from either HEK 293S cells expressing the cloned human CB₁ receptor (hCB₁: clone #24) or Sf9 cells, using the baculovirus system, expressing the cloned human CB₂ receptor (hCB₂). The membranes are thawed at 37° C., passed 3 times through a 23-gauge blunt-end needle, diluted in the cannabinoid binding buffer (50 mM Tris, 2.5 mM EDTA, 5 mM MgCl₂, and 0.5 mg/mL BSA fatty acid free, pH 7.4) and aliquots containing the appropriate amount of protein are distributed in 96-well plates. The IC₅₀ of the compounds of the present invention at hCB₁ and hCB₂ are evaluated from 10-point dose-response curves done with ³H-CP55,940 at 20000 to 25000 dpm per well (0.17-0.21 nM) in a final volume of 300 μl. The total and non-specific binding are determined in the absence and presence of 0.2 μM of HU210 respectively. The plates are vortexed and incubated for 60 minutes at room temperature, filtered through Unifilters GF/B (pre-soaked in 0.1% polyethyleneimine) with the Packard harvester using 3 mL of wash buffer (50 mM Tris, 5 mM MgCl₂, 0.5 mg BSA pH 7.0). The filters are dried for 1 hour at 55° C. The radioactivity (cpm) is counted in a TopCount (Packard) after adding 65 μl/well of MS-20 scintillation liquid.

hCB₁ GTPγS Binding

Cloned human CB₁ receptor from Perkin-Elmer (hCB₁) are thawed at 37° C., passed 3 times through a 23-gauge blunt-end needle and diluted in the GTPγS binding buffer (50 mM Hepes, 20 mM NaOH, 100 mM NaCl, 1 mM EDTA, 5 mM MgCl₂, pH 7.4, 0.1% BSA). The EC₅₀ and Eₘₐₓ of the compounds of the present invention are evaluated from 10-point dose-response curves done in 3001 with the appropriate amount of membrane protein and 100000-130000 dpm of GTPγ³⁵S per well (0.11-0.14 nM). The basal and maximal stimulated binding is determined in absence and presence of 10 μM Win 55,212-2. The membranes are pre-incubated for 5 minutes with 112.5 μM GDP prior to distribution in plates (30 μM GDP final). The plates are vortexed and incubated for 60 minutes at room temperature, filtered on Unifilters GF/B (pre-soaked in water) with the Packard harvester using 3 ml of wash buffer (50 mM Tris, 5 mM MgCl₂, 50 mM NaCl, pH 7.0). The filters are dried for 1 hour at 55° C. The radioactivity (cpm) is counted in a TopCount (Packard) after adding 65 μl/well of MS-20 scintillation liquid.

Based on the above assays, the dissociation constant (Ki) for a particular compound of the present invention towards a particular receptor is determined using the following equation:

Ki=IC₅₀/(1+[rad]/Kd),

wherein IC₅₀ is the concentration of the compound of the present invention at which 50% displacement has been observed;
[rad] is a standard or reference radioactive ligand concentration at that moment; Kd is the dissociation constant of the radioactive ligand towards the particular receptor.

Using the above-mentioned assays, the Ki towards human CB₁ receptors for most compounds of the present invention is measured to be in the range of 2-5000 nM. The EC₅₀ towards human CB₁ receptors for most compounds of the present invention is measured to be in the range of about 2-5500 nM. The Eₘₐₓ towards human CB₁ receptors for most compounds of the invention is measured to be in the range of about 0-150%.

The following table shows certain biological activities for some of the exemplified compounds.

| | Compound | | |
|---|---|---|---|
| | Ki hCB₁ | EC₅₀ hCB₁ | Eₘₐₓ hCB₁ |
| | (nM) | (nM) | (%) |
| Example 6 | 52 | 80 | 88 |
| Example 7 | 59 | 120 | 110 |
| Example 14 | 3.2 | 7.3 | 110 |
| Example 15 | 13 | 22 | 120 |
| Example 27 | 35 | 42 | 94 |
| Example 29 | 5.4 | 7.1 | 80 |
| Example 35 | 53 | 99 | 93 |
| Example 44 | 270 | 240 | 140 |
| Example 50 | 51 | 46 | 48 |
| Example 56 | 260 | 380 | 88 |

### Screening for Compounds Active Against TLESR

Adult Labrador retrievers of both genders, trained to stand in a Pavlov sling, are used. Mucosa-to-skin esophagostomies are formed and the dogs are allowed to recover completely before any experiments are done.

### Motility Measurement

In brief, after fasting for approximately 17 h with free supply of water, a multilumen sleeve/sidehole assembly (Dentsleeve, Adelaide, South Australia) is introduced through the esophagostomy to measure gastric, lower esophageal sphincter (LES) and esophageal pressures. The assembly is perfused with water using a low-compliance manometric perfusion pump (Dentsleeve, Adelaide, South Australia). An air-perfused tube is passed in the oral direction to measure swallows, and an antimony electrode monitored pH, 3 cm above the LES. All signals are amplified and acquired on a personal computer at 10 Hz.

When a baseline measurement free from fasting gastric/LES phase III motor activity has been obtained, placebo (vehicle) or test compound is administered intravenously (i.v., 0.5 ml/kg) in a foreleg vein or orally (p.o., 2 ml/kg). 10 min after i.v. administration or 30 min after p.o. administration, a nutrient meal (10% peptone, 5% D-glucose, 5% Intralipid, pH 3.0) is infused into the stomach through the central lumen of the assembly at 100 ml/min to a final volume of 30 ml/kg. Immediately following the meal, air is insufflated at 40 ml/min. In an alternative model (Barostat model), the infusion of the nutrient meal is followed by air infusion at a rate of 500 ml/min until a intragastric pressure of 10±1 mmHg is obtained. The pressure is then maintained at this level throughout the experiment using the infusion pump for further air infusion or for venting air from the stomach. The experimental time from start of nutrient infusion to end of air insufflation is 45 min. The procedure has been validated as a reliable means of triggering TLESRs.

TLESRs is defined as a decrease in lower esophageal sphincter pressure (with reference to intragastric pressure) at a rate of >1 mmHg/s. The relaxation should not be preceded by a pharyngeal signal <2 s before its onset in which case the relaxation is classified as swallow-induced. The pressure difference between the LES and the stomach should be less than 2 mmHg, and the duration of the complete relaxation longer than 1 s.

Inhibition of the number of TLESRs was calculated with regard to control experiments for each dog.

### EXAMPLES

The present invention will further be described in more detail by the following Examples, which describe methods whereby compounds of the present invention may be prepared, purified, analyzed and biologically tested, and which are not to be construed as limiting the present invention.

If not otherwise stated the compounds was purified with flash chromatography using a Horizon/Biotage system with prepacked Biotage Si 25+M columns and heptane/ethyl acetate (1:0 to 1:2) as eluent.

### Example 1

### Methyl[(6-{[(cyclohexylmethyl)amino]carbonyl}-5-{[4-(1H-1,2,3-triazol-1-ylmethyl)-1-naphthoyl]amino}pyridin-2-yl)oxy]acetate

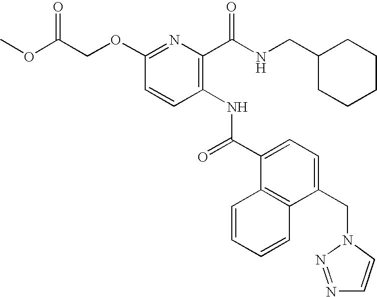

### Example 1A

### Methyl[(6-{[(cyclohexylmethyl)amino]carbonyl}-5-{[4-(1H-1,2,3-triazol-1-ylmethyl)-1-naphthoyl]amino}pyridin-2-yl)oxy]acetate

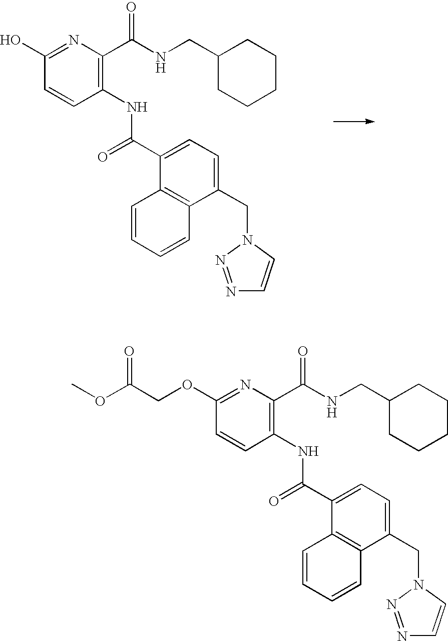

A mixture of N-(cyclohexylmethyl)-6-hydroxy-3-{[4-(1H-1,2,3-triazol-1-ylmethyl)-1-naphthoyl]amino}pyridine-2-carboxamide prepared in Example 1B (20 mg, 0.04 mmol), silver carbonate (57 mg, 0.21 mmol) and methyl bromoacetate (19 mg, 0.12 mmol) in acetonitrile (2.5 ml) was refluxed for 50 min. The reaction mixture was allowed to reach room temperature, and was then diluted with dichloromethane and filtered. The filtrate was washed with water, dried and evaporated under reduced pressure. The residue was purified by column chromatography on silica gel using CH₂Cl₂/MeOH as eluent to give the title compound (14 mg, 63%).

¹H-NMR (500 MHz, CDCl₃) δ (ppm) 0.94-104 (m, 2H), 1.12-1.31 (m, 3H), 1.51-1.61 (m, 1H), 1.62-1.80 (m, 5H), 3.21 (t, J=6.6 Hz, 2H), 3.75 (s, 3H), 4.80 (s, 2H), 6.06 (s, 2H), 7.16 (d, J=9.4 Hz, 1H), 7.39 (d, J=1 Hz, 1H), 7.43 (d, J=7.0 Hz, 1H), 7.55-7.62 (m, 2H), 7.69 (d, J=1 Hz, 1H), 7.84 (d, J=7.5 Hz, 1H), 7.95 (t, J=6.1 Hz, 1H), 8.01 (dd, J=7.0, 2.35 Hz, 1H), 8.54 (dd, J=7.0, 2.4 Hz, 1H), 9.41 (d, J=8.9 Hz, 1H), 12.66 (s, 1H). MS (ESI) (M+H)⁺ 485.15.

### Example 1B

### N-(Cyclohexylmethyl)-6-hydroxy-3-{[4-(1H-1,2,3-triazol-1-ylmethyl)-1-naphthoyl]amino}pyridine-2-carboxamide

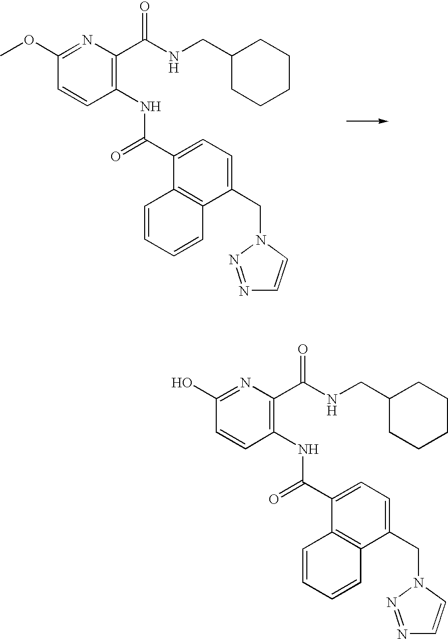

A mixture of N-(cyclohexylmethyl)-6-methoxy-3-{[4-(1H-1,2,3-triazol-1-ylmethyl)-1-naphthoyl]amino}pyridine-2-carboxamide prepared in Example 1C (0.29 g, 0.58 mmol) and pyridine hydrochloride (7.3 g, 63.17 mmol) was heated at 150° C. for 25 min. Water was added at RT. The formed precipitate was collected, washed with water, dried and then purified by preparative HPLC using acetonitrile and ammonium acetate buffer (25:75 to 95:5) to give 193 mg (69%) of the title compound.

¹H-NMR (500 MHz, CD₃OD) δ (ppm) 0.92-1.02 (m, 2H), 1.12-1.30 (m, 3H), 1.50-1.60 (m, 1H), 1.62-1.78 (m, 5H), 3.15 (d, J=7.0 Hz, 2H), 6.19 (s, 2H), 6.96 (d, J=8.9 Hz, 1H), 7.47 (d, J=7.0 Hz, 1H), 7.60-7.66 (m, 2H), 7.73 (d, J=0.9 Hz, 1H), 7.84 (d, J=7.0 Hz, 1H), 7.94 (d, J=0.9 Hz, 1H), 8.19-8.24 (m, 1H), 8.43-8.48 (m, 1H), 9.12 (d, J=8.9 Hz, 1H). MS (ESI) (M+H)⁺ 485.15.

### Example 1C

### N-(cyclohexylmethyl)-6-methoxy-3-{[4-(1H-1,2,3-triazol-1-ylmethyl)-1-naphthoyl]amino}pyridine-2-carboxamide

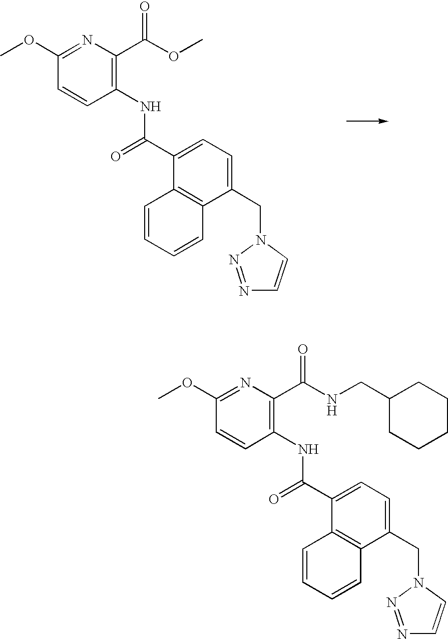

A solution of methyl 6-methoxy-3-{[4-(1H-1,2,3-triazol-1-ylmethyl)-1-naphthoyl]amino}pyridine-2-carboxylate prepared in Example 1D (0.5 g, 1.2 mmol) and cyclohexanemethylamine (0.41 g, 3.6 mmol) in DMF (3 ml) was heated at 80° C. for 40 min. The solution was evaporated under reduced pressure, and the residue was dissolved in dichloromethane. After addition of water (50 ml) and 2 N HCl (aq) (13 ml), the organic phase was separated, washed with NaHCO₃ (aq, sat) and brine, and then dried and evaporated under reduced pressure. The residue was purified by preparative HPLC using acetonitrile and ammonium acetate buffer (30:70 to 95:5) as eluent to give 517 mg (86%) of N-(cyclohexylmethyl)-6-methoxy-3-{[4-(1H-1,2,3-triazol-1-ylmethyl)-1-naphthoyl]amino}pyridine-2-carboxamide.

¹H-NMR (600 MHz, CDCl₃) δ (ppm) 0.93-1.02 (m, 2H), 1.09-1.27 (m, 3H), 1.50-1.58 (m, 1H), 1.62-1.78 (m, 5H), 3.22 (t, J=6.7 Hz, 2H), 3.94 (s, 3H), 6.04 (s, 2H), 7.01 (d, J=9.1 Hz, 1H), 7.36 (s, 1H), 7.41 (d, J=7.2 Hz, 1H), 7.53-7.60 (m, 2H), 7.66 (s, 1H), 7.83 (d, J=7.2 Hz, 1H), 7.98 (d, J=7.8 Hz, 1H), 8.23 (t, J=6.5 Hz, 1H), 8.53 (d, J=8.5 Hz, 1H), 9.31 (d, J=9.1 Hz, 1H), 12.62 (s, 1H). MS (ESI) (M+H)⁺ 499.12.

### Example 1D

### Methyl 6-methoxy-3-{[4-(1H-1,2,3-triazol-1-ylmethyl)-1-naphthoyl]amino}pyridine-2-carboxylate

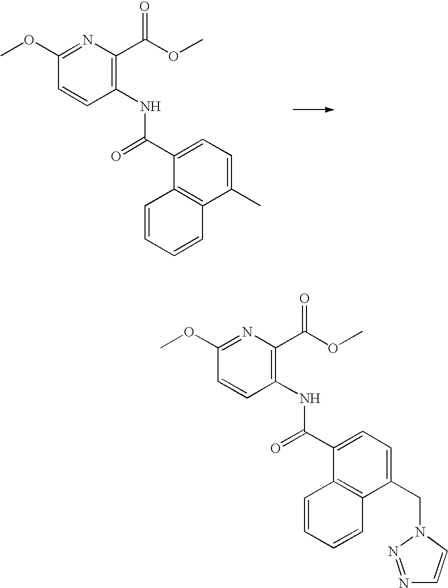

To a mixture of methyl 6-methoxy-3-[(4-methyl-1-naphthoyl)amino]pyridine-2-carboxylate prepared in Example 1E (1.8 g, 5.14 mmol) in CCl₄ (100 ml) was added N-bromosuccinimide (0.96 g, 5.39 mmol) and benzoyl peroxide (0.125 g, 0.51 mmol). The reaction mixture was refluxed for 1.5 h under nitrogen. DMF (2.5 ml) and 1,2,3-triazole (2.98 ml, 51.4 mmol) was added, and the reaction mixture was refluxed overnight. After removal of solvents, the residue was suspended in cold water. The formed precipitate was collected, washed with water, air dried and purified by column chromatography on silica gel using first CH₂Cl₂ and then CH₂Cl₂/MeOH (100:1) as eluent to give 1.55 g (72%) of methyl 6-methoxy-3-{[4-(1H-1,2,3-triazol-1-ylmethyl)-1 naphthoyl]amino}pyridine-2-carboxylate. MS (ESI) (M+H)⁺ 418.13.

### Example 1E

### Methyl 6-methoxy-3-[(4-methyl-1-naphthoyl)amino]pyridine-2-carboxylate

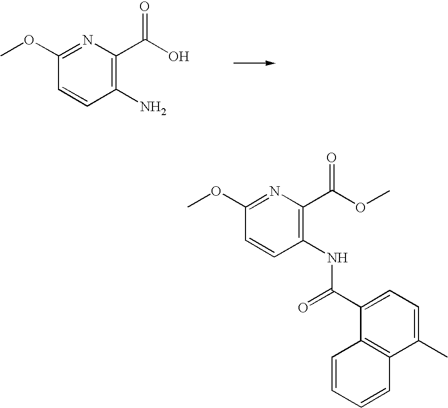

To a solution of 3-Amino-6-methoxy-pyridine-2-carboxylic acid prepared in Example 1F (1.78 g, 10.6 mmol) in anhydrous DMF (30 ml) was added DIPEA (11.07 ml, 63.6 mmol) and 4-methyl-1-naphthalenecarbonyl chloride (2.65 g, 12.95 mmol) under nitrogen. After stirred for 1 h at RT, and for 1 h at 50° C., K₂CO₃ (2.2 g, 15.9 mmol) was then added to the reaction mixture followed by the addition of MeI (3.3 ml, 53 mmol) in portions at RT. After stirred overnight, the reaction mixture was condensed, and the residue was suspended in water, and the crystals filtered, washed with water, ethanol and air-dried. The crude product (2.7 g) was suspended in ethyl acetate/methanol, and the crystals filtered, washed with methanol, ether and air-dried to give 2 g (54%) of methyl 6-methoxy-3-[(4-methyl-1-naphthoyl)amino]pyridine-2-carboxylate. MS (ESI) (M+H)⁺ 351.10.

### Example 1F

### 3-Amino-6-methoxy-pyridine-2-carboxylic Acid

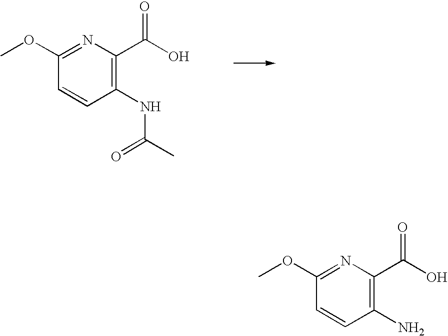

3-(Acetylamino)-6-methoxypyridine-2-carboxylic acid, obtained according to the procedure of Goldberg et al. [Besly; Goldberg; JCSOA9; J. Chem. Soc.; 2448, 2455](7.96 g, 37.88 mmol) was refluxed for 80 min with 2.5 N NaOH (aq, sat) (80 ml). The solution was adjusted to pH 4 with 4 N HCl (aq) at 0° C. The formed precipitate was collected, washed with cold water and air-dried to give 5.65 g (89%) of 3-Amino-6-methoxy-pyridine-2-carboxylic acid. MS (ESI) (M+H)⁺ 169.14.

### Example 2

### Methyl[(6-{[(cyclobutylmethyl)amino]carbonyl}-5-{[4-(1H-1,2,3-triazol-1-ylmethyl)-1-naphthoyl]amino}pyridin-2-yl)oxy]acetate

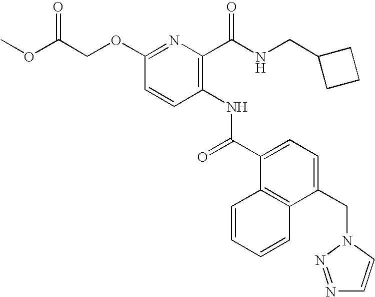

### Example 2A

### Methyl[(6-{[(cyclobutylmethyl)amino]carbonyl)-5-([4-(1H-1,2,3-triazol-1-ylmethyl)-1-naphthoyl]amino}pyridin-2-yl)oxy]acetate

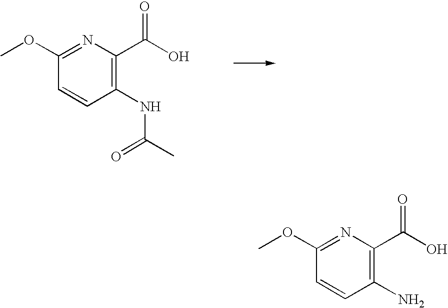

Following the procedure disclosed in Example 1A, using N-(cyclobutylmethyl)-6-hydroxy-3-{[4-(1H-1,2,3-triazol-1-ylmethyl)-1-naphthoyl]amino}pyridine-2-carboxamide (300 mg, 0.66 mmol), prepared in Example 2B and methyl bromoacetate (302 mg, 1.97 mmol) provided the title compound after purification by column chromatography on silica gel using CH₂Cl₂/MeOH (100:1.5) as eluent (250 mg, 72%).

¹H NMR (300 MHz, CDCl₃) δ (ppm) 1.60-1.80 (m, 2H), 1.81-2.0 (m, 2H), 2.01-2.20 (m, 2H), 2.47-2.65 (m, 1H), 3.38 (t, J=6.9 Hz, 2H), 3.74 (s, 3H), 4.77 (s, 2H), 6.04 (s, 2H), 7.14 (d, J=9.1 Hz, 1H), 7.38 (s, 1H), 7.40 (d, J=7.4 Hz, 1H), 7.51-7.62 (m, 2H), 7.66 (s, 1H), 7.79-7.92 (m, 2H), 7.94-8.03 (m, 1H), 8.47-8.56 (m, 1H), 9.38 (d, J=9.2 Hz, 1H), 12.64 (s, 1H). MS (ESI) (M+H)⁺ 529.04.

### Example 2B

### N-(cyclobutylmethyl)-6-hydroxy-3-{[4-(1H-1,2,3-triazol-1-ylmethyl)-1-naphthoyl]amino}pyridine-2-carboxamide

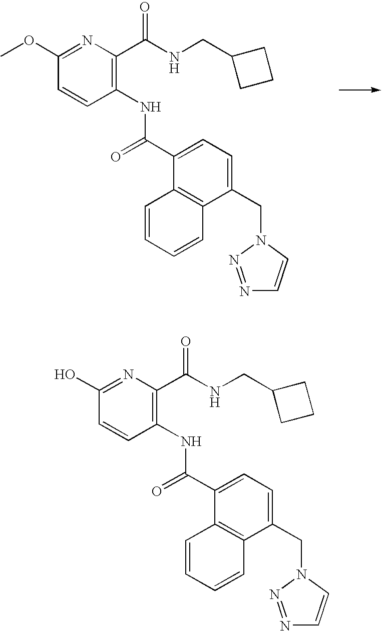

A mixture of N-(cyclobutylmethyl)-6-methoxy-3-{[4-(1H-1,2,3-triazol-1-ylmethyl)-1-naphthoyl]amino}pyridine-2-carboxamide (300 mg, 0.64 mmol) and pyridine hydrochloride (7 g, 60.57 mmol) was heated at 150° C. for 30 min. Water was added at RT. The formed precipitate was collected, washed with water, dried and purified by preparative HPLC using acetonitrile and ammonium acetate buffer (20:80 to 95:5) to give 223 mg (77%) of title compound.

¹H-NMR (300 MHz, CD₃OD) δ (ppm) 1.66-181 (m, 2H), 1.82-1.94 (m, 2H), 2.0-2.14 (m, 2H), 2.47-2.65 (m, 1H), 3.3-2.37 (m, 2H), 6.19 (s, 2H), 6.90 (d, J=9.24 Hz, 1H), 7.46 (d, J=7.22 Hz, 1H), 7.57-7.68 (m, 2H), 7.73 (s, 1H), 7.84 (d, J=7.39 Hz, 1H), 7.94 (s, 1H), 8.16-8.26 (m, 1H), 8.41-8.50 (m, 1H), 9.11 (d, J=9.06 Hz, 1H). MS (ESI) (M+H)⁺ 357.

### Example 2C

### 6-methoxy-N-(tetrahydro-2H-pyran-4-ylmethyl)-3-{[4-(1H-1,2,3-triazol-1-ylmethyl)-1-naphthoyl]amino}pyridine-2-carboxamide

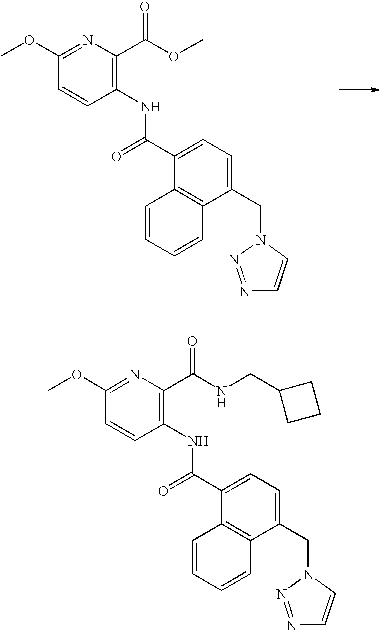

A solution of methyl 6-methoxy-3-{[4-(1H-1,2,3-triazol-1-ylmethyl)-1-naphthoyl]amino}pyridine-2-carboxylate (1 g, 2.4 mmol, see Example 1D) and cyclobutyl methylamine (490 mg, 5.75 mmol) in DMF (7 ml) was heated at 80° C. for 140 min. More cyclobutyl methylamine (240 mg, 2.82 mmol) was added and the reaction mixture was heated at 80° C. for additional 90 min. The solution was evaporated under reduced pressure and the residue was purified by preparative HPLC using acetonitrile and ammonium acetate buffer (20:80 to 95:5) as eluent to give 980 mg (87%) of 6-methoxy-N-(tetrahydro-2H-pyran-4-ylmethyl)-3-{[4-(1H-1,2,3-triazol-1-ylmethyl)-1-naphthoyl]amino}pyridine-2-carboxamide. MS (ESI) (M+H)⁺ 471.04.

### Example 3

### [(6-{[(Cyclobutylmethyl)amino]carbonyl}-5-{[4-(1H-1,2,3-triazol-1-ylmethyl)-1-naphthoyl]amino}pyridin-2-yl)oxy]acetic Acid

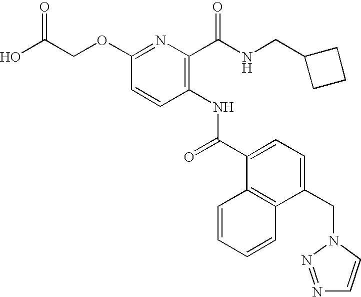

To a suspension of methyl [(6-{[(cyclobutylmethyl)amino]carbonyl}-5-{[4-(1H-1,2,3-triazol-1-ylmethyl)-1-naphthoyl]amino}pyridin-2-yl)oxy]acetate, which was prepared in Example 2A, (150 mg, 0.28 mmol) in methanol/ethanol (10 ml, 1:1) was added a solution of NaOH (33 mg dissolved in 0.6 ml water). The reaction mixture was stirred at room temperature for 45 min and then quenched by adding acetic acid (150 ml). The solution was evaporated under reduced pressure, and the residue was dissolved in dichloromethane, washed with water and brine and then evaporated under reduced pressure to give the title compound (139 mg, 95%).

¹H NMR (600 MHz, CD₃COD) δ (ppm) 1.72-1.80 (m, 2H), 1.84-1.95 (m, 2H), 2.04-2.12 (m, 2H), 2.53-2.62 (m, 1H), 3.33-3.36 (m, 2H), 4.89 (s, 2H), 6.19 (s, 2H), 7.19 (d, J=9.1 Hz, 1H), 7.45 (d, J=7.3 Hz, 1H), 7.59-7.65 (m, 2H), 7.73 (s, 1H), 7.85 (d, J=7.3 Hz, 1H), 7.94 (s, 1H), 8.18-8.24 (m, 1H), 8.38 (t, J=5.5 Hz, 1H), 8.44-8.49 (m, 1H), 9.25 (d, J=9.1 Hz, 1H). MS (ESI) (M+H)⁺ 515.

### Example 4

### 6-(2-Amino-2-oxoethoxy)-N-(cyclobutylmethyl)-3-{[4-(1H-1,2,3-triazol-1-ylmethyl)-1-naphthoyl]amino}pyridine-2-carboxamide

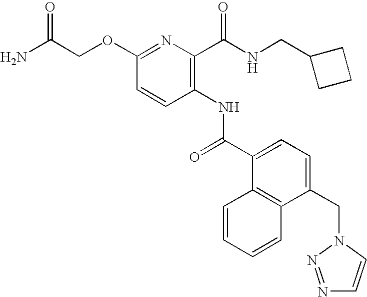

To a solution of [(6-{[(cyclobutylmethyl)amino]carbonyl}-5-{[4-(1H-1,2,3-triazol-1-ylmethyl)-1-naphthoyl]amino}pyridin-2-yl)oxy]acetic acid, which was prepared in is Example 3, (50 mg, 0.097 mmol) in anhydrous DMF (2 ml) were added TBTU (47 mg, 0.15 mmol), DIPEA (25 mg, 0.19 mmol) and ammonium chloride (30 mg, 0.56 mmol) under nitrogen. The reaction mixture was stirred for 1 h at room temperature. Water was added and the formed precipitate was collected, washed with water and air-dried. The solid was dissolved in dichloromethane/methanol and filtered. The solution was evaporated under reduced pressure and the residue suspended in ether. The solid was collected, washed with ether and dried in vacuo to give the title compound (49 mg, 98%).

¹H NMR (300 MHz, CDCl₃) δ (ppm) 1.69-1.81 (m, 2H), 1.82-2.0 (m, 2H), 2.03-2.18 (m, 2H), 2.50-2.65 (m, 1H), 3.40 (t, J=6.9 Hz), 4.74 (s, 2H), 5.47 (br s, 1H), 6.06 (s, 2H), 6.22 (br s, 1H), 7.13 (d, J=9.2 Hz, 1H), 7.40 (s, 1H), 7.44 (d, J=7.2 Hz, 1H), 7.53-7.65 (m, 2H), 7.69 (s, 1H), 7.85 (d, J=7.2 Hz, 1H), 7.95-8.08 (m, 2H), 8.50-8.59 (m, 1H), 9.43 (d, J=9.1 Hz, 1H), 12.70 (s, 1H). MS (ESI) (M+H)⁺ 514.

### Example 5

### N-(cyclobutylmethyl)-6-[2-(methylamino)-2-oxoethoxy]-3-{[4-(1H-1,2,3-triazol-1-ylmethyl)-1-naphthoyl]amino}pyridine-2-carboxamide

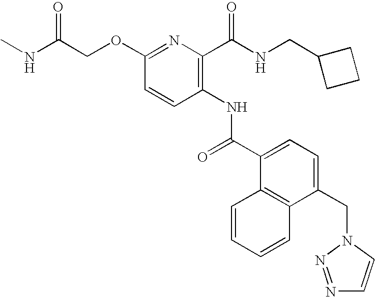

Following the procedure disclosed in Example 4, using [(6-{[(cyclobutylmethyl)amino]carbonyl}-5-{[4-(1H-1,2,3-triazol-1-ylmethyl)-1-naphthoyl]amino}pyridin-2-yl)oxy]acetic acid, which was prepared in Example 3, (50 mg, 0.097 mmol), and methylamine hydrochloride (32 mg, 0.47 mmol) provided the title compound after workup (51 mg, 99%).

¹H NMR (600 MHz, CDCl₃) δ (ppm) 1.60-1.79 (m, 2H), 1.81-1.96 (m, 2H). 2.02-2.13 (m, 2H), 2.52-2.64 (m, 1H), 2.86 (br s, 3H), 3.32-3.42 (m, 2H), 4.73 (s, 2H), 6.06 (s, 2H), 6.29 (br s, 1H), 7.09 (d, J=8.7 Hz, 1H), 7.37 (s, 1H), 7.42 (d, J=6.2 Hz, 1H), 7.52-7.62 (m, 2H), 7.67 (s, 1H), 7.83 (d, J=6.7 Hz, 1H), 7.93-8.06 (m, 2H), 8.52 (d, J=7.2 Hz, 1H), 9.38 (d, J=8.6 Hz, 1H), 12.70 (s, 1H). MS (ESI) (M+H)⁺ 528.

### Example 6

### N-(cyclobutylmethyl)-6-[2-(dimethylamino)-2-oxoethoxy]-3-{[4-(1H-1,2,3-triazol-1-ylmethyl)-1-naphthoyl]amino}pyridine-2-carboxamide

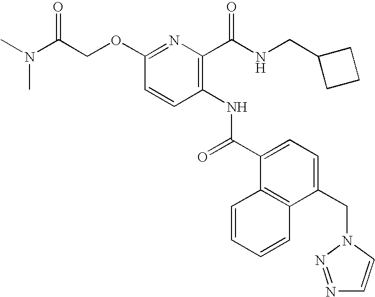

Following the procedure disclosed in Example 4, using [(6-{[(cyclobutylmethyl)amino]carbonyl}-5-{[4-(1H-1,2,3-triazol-1-ylmethyl)-1-naphthoyl]amino}pyridin-2-yl)oxy]acetic acid, which was prepared in Example 3, (19 mg, 0.037 mmol), and dimethylamine hydrochloride (12 mg, 0.15 mmol) provided the title compound after workup (18 mg, 90%).

¹H NMR (600 MHz, CDCl₃) δ (ppm) 1.70-1.80 (m, 2H), 1.82-1.95 (m, 2H), 2.03-2.12 (m, 2H), 2.58-2.68 (m, 1H), 2.88 (s, 3H), 3.07 (s, 3H), 3.39 (t, J=6.3 Hz, 2H), 4.82 (s, 2H), 6.04 (s, 2H), 7.06 (d, J=9.1 Hz, 1H), 7.37 (s, 1H), 7.41 (d, J=7.2 Hz, 1H), 7.53-7.60 (m, 2H), 7.67 (s, 1H), 7.83 (d, J=7.0 Hz, 1H), 7.97 (d, J=8.0 Hz, 1H), 8.41-8.44 (m, 1H), 8.51 (d, J=8.0 Hz, 1H), 9.34 (d, J=9.0 Hz, 1H), 12.68 (s, 1H). MS (ESI) (M+H)⁺ 542.02.

### Example 7

### N-(cyclobutylmethyl)-6-{2-[(2-hydroxyethyl)amino]-2-oxoethoxy}-3-{[4-(1H-1,2,3-triazol-1-ylmethyl)-1-naphthoyl]amino}pyridine-2-carboxamide

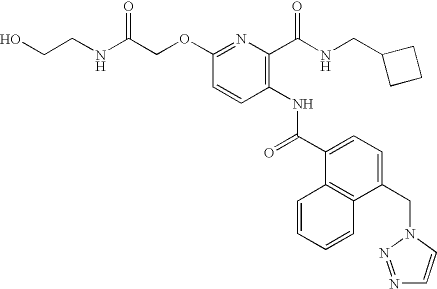

Following the procedure disclosed in Example 4, using [(6-{[(cyclobutylmethyl)amino]carbonyl}-5-{[4-(1H-1,2,3-triazol-1-ylmethyl)-1-naphthoyl]amino}pyridin-2-yl)oxy]acetic acid, which was obtained from Example 3, (50 mg, 0.097 mmol), and ethanolamine (17 mg, 0.28 mmol) provided the title compound after workup (53 mg, 98%).

¹H NMR (300 MHz, CDCl₃) δ (ppm) 1.60-1.82 (m, 2H), 1.84-1.98 (m, 2H), 2.04-2.20 (m, 2H), 2.51-2.67 (m, 1H), 3.34-3.44 (m, 2H), 3.45-3.54 (m, 2H), 3.69-3.77 (m, 2H), 4.76 (s, 2H), 6.06 (s, 2H), 6.66-6.77 (m, 1H), 7.13 (d, J=9.2 Hz, 1H), 7.40 (s, 1H), 7.43 (d, J=7.2 Hz, 1H), 7.55-7.64 (m, 2H), 7.69 (s, 1H), 7.85 (d, J=7.2 Hz, 1H), 7.55-7.64 (m, 2H), 7.69 (s, 1H), 7.85 (d. J=9.1 Hz, 1H), 7.95-8.07 (m, 2H), 8.50-8.59 (m, 1H), 9.42 (d, J=9.1 Hz, 1H), 12.70 (s, 1H). MS (ESI) (M+H)⁺ 558.

### Example 8

### 6-{[(Cyclobutylmethyl)amino]carbonyl}-5-{[4-(1H-1,2,3-triazol-1-ylmethyl)-1-naphthoyl]amino}pyridin-2-yl Ethanesulfonate

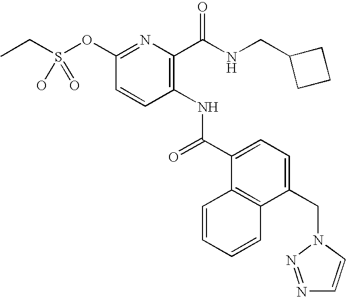

Following the procedure disclosed in Example 1A, using N-(cyclobutylmethyl)-6-hydroxy-3-{[4-(1H-1,2,3-triazol-1-ylmethyl)-1-naphthoyl]amino}pyridine-2-carboxamide (50 mg, 0.11 mmol), prepared in Example 2B, and ethanesulphonyl chloride (42 mg, 0.33 mmol) provided the title compound after purification by column chromatography on silica gel using CH₂Cl₂/MeOH (100:1.5) as eluent (50 mg, 83%).

¹H NMR (300 MHz, CDCl₃) δ (ppm) 1.62 (t, J=7.4 Hz, 3H), 1.66-2.01 (m, 4H), 2.02-2.18 (m, 2H), 2.49-2.66 (m, 1H), 3.35-3.52 (m, 4H), 6.07 (s, 2H), 7.34-7.45 (m, 3H), 7.54-7.66 (m, 2H), 7.69 (s, 1H), 7.85 (d, J=7.2 Hz, 1H), 7.92-8.08 (m, 2H), 8.48-8.59 (m, 1H), 9.57 (d, J=9.1 Hz, 1H), 12.81 (s, 1H). MS (ESI) (M+H)⁺ 549.06

### Example 9

### 6-{[(Cyclobutylmethyl)amino]carbonyl}-5-{[4-(1H-1,2,3-triazol-1-ylmethyl)-1-naphthoyl]amino}pyridin-2-yl 3,3,3-trifluoropropane-1-sulfonate

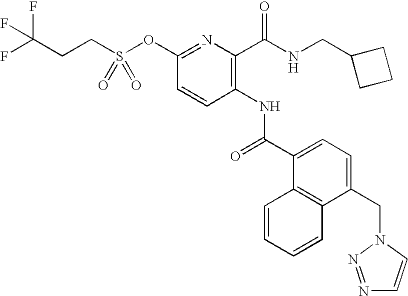

Following the procedure disclosed in Example 1A, using N-(cyclobutylmethyl)-6-hydroxy-3-{[4-(1H-1,2,3-triazol-1-ylmethyl)-1-naphthoyl]amino}pyridine-2-carboxamide (37 mg, 0.081 mmol), prepared in Example 2B, and 3,3,3-trifluoropropylsulphonyl chloride (32 mg, 0.16 mmol) provided the title compound after purification by column chromatography on silica gel using CH₂Cl₂/MeOH (100:1.5) as eluent (30 mg, 60%).

¹H NMR (600 MHz, CDCl₃) δ (ppm) 1.70-1.72 (m, 2H), 1.85-1.97 (m, 2H), 2.05-2.14 (m, 2H), 2.51-2.60 (m, 1H), 2.88-2.98 (m, 2H), 3.39-3.45 (m, 2H), 3.66-3.72 (m, 2H), 6.07 (s, 2H), 7.39-7.45 (m, 3H), 7.58-7.65 (m, 2H), 7.70 (s, 1H), 7.80-7.90 (m, 2H), 8.01-8.08 (m, 1H), 8.52-8.58 (m, 1H), 9.62 (d, J=9.1 Hz, 1H), 12.88 (s, 1H). MS (ESI) (M+H)⁺ 616.99.

### Example 10

### 6-{[(Tetrahydro-2H-pyran-4-ylmethyl)amino]carbonyl}-5-{[4-(1H-1,2,3-triazol-1-ylmethyl)-1-naphthoyl]amino}pyridin-2-yl 3,3,3-trifluoropropane-1-sulfonate

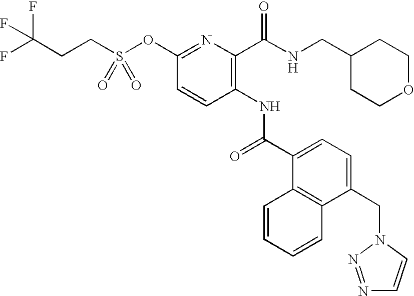

### Example 10A

### 6-{[(Tetrahydro-2H-pyran-4-ylmethyl)amino]carbonyl}-5-{[4-(1H-1,2,3-triazol-1-ylmethyl)-1-naphthoyl]amino}pyridin-2-yl 3,3,3-trifluoropropane-1-sulfonate

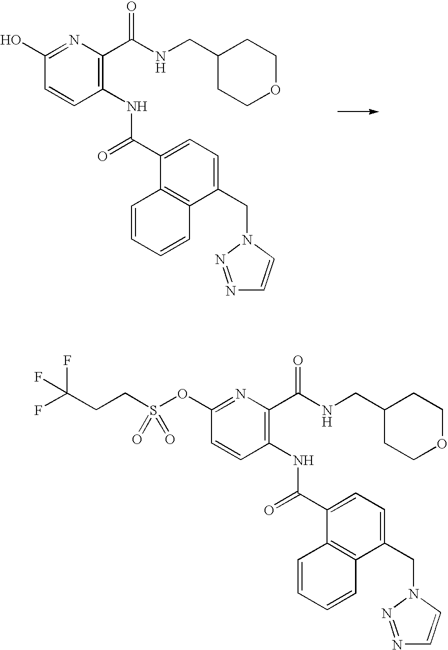

Following the procedure disclosed in Example 1A, using 6-hydroxy-N-(tetrahydro-2H-pyran-4-ylmethyl)-3-{[4-(1H-1,2,3-triazol-1-ylmethyl)-1-naphthoyl]amino}pyridine-2-carboxamide (50 mg, 0.103 mmol), prepared in Example 10B and 3,3,3-trifluoropropylsulphonyl chloride (40 mg, 0.21 mmol) provided the title compound after purification by column chromatography on silica gel using CH₂Cl₂/MeOH (100:1.5) as eluent (23 mg, 35%).

¹H NMR (600 MHz, CDCl₃) δ (ppm) 1.34-1.40 (m, 2H), 1.60-1.66 (m, 2H), 1.78-1.88 (m, 1H), 2.88-2.98 (m, 2H), 3.28-3.40 (m, 4H), 3.65-3.71 (m, 2H), 3.95-4.01 (m, 2H), 6.08 (s, 2H), 7.40-7.45 (m, 3H), 7.59-7.66 (m, 2H), 7.71 (s, 1H), 7.86 (d, J=7.3 Hz, 1H), 7.94-7.99 (m, 1H), 8.02-8.08 (m, 1H), 8.52-8.57 (m, 1H), 9.64 (d, J=9.0 Hz, 1H), 12.81 (s, 1H). MS (ESI) (M+H)⁺ 646.99.

### Example 10B

### 6-hydroxy-N-(tetrahydro-2H-pyran-4-ylmethyl)-3-{[4-(1H-1,2,3-triazol-1-ylmethyl)-1-naphthoyl]amino}pyridine-2-carboxamide

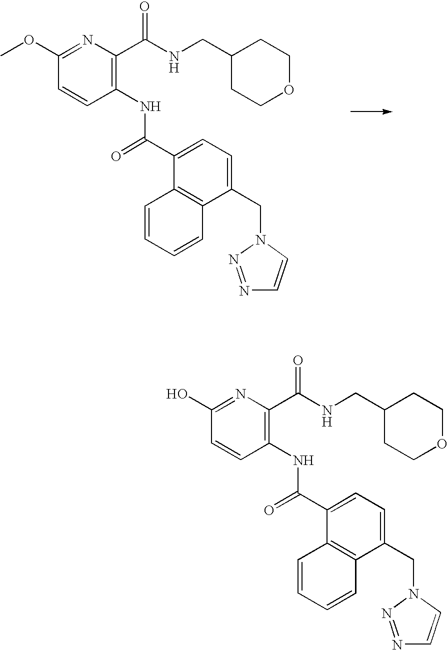

A mixture of 6-methoxy-N-(tetrahydro-2H-pyran-4-ylmethyl)-3-{[4-(1H-1,2,3-triazol-1-ylmethyl)-1-naphthoyl]amino}pyridine-2-carboxamide, prepared in 10C, (145 mg, 0.3 mmol) and pyridine hydrochloride (3.8 g, 32.88 mmol) was heated at 150° C. for 27 min. Water was added at RT. The formed precipitate was collected, washed with water, dried and purified by preparative HPLC using acetonitrile and ammonium acetate buffer (20:80 to 95:5) to give 113 mg (80%) of the title compound.

¹H-NMR (300 MHz, CD₃OD): 1.22-1.40 (m, 2H), 1.57-1.69 (m, 2H), 1.74-1.92 (m, 1H), 3.20-3.42 (m, 4H), 3.85-3.96 (m, 2H), 6.20 (s, 2H), 6.96 (d, J=9.07 Hz, 1H), 7.46 (d, J=7.39 Hz, 1H), 7.58-7.69 (m, 2H), 7.74 (s, 1H), 7.85 (d, J=7.22 Hz, 1H), 7.95 (s, 1H), 8.18-8.27 (m, 1H), 8.41-8.50 (m, 1H), 9.12 (d, J=9.07 Hz, 1H). MS (ESI) (M+H)⁺ 487.12.

### Example 10C

### 6-methoxy-N-(tetrahydro-2H-pyran-4-ylmethyl)-3-{[4-(1H-1,2,3-triazol-1-ylmethyl)-1-naphthoyl]amino}pyridine-2-carboxamide

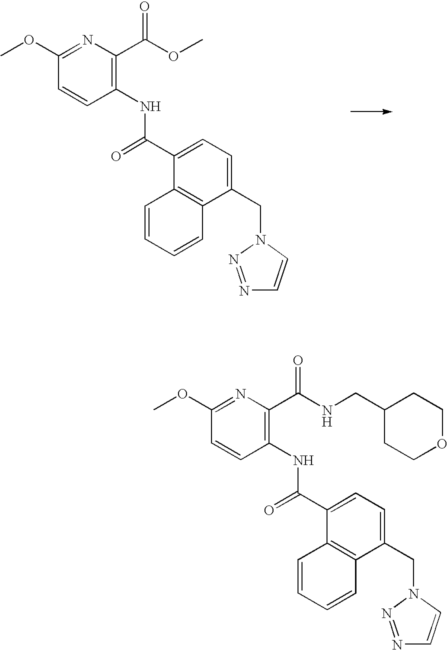

A solution of methyl 6-methoxy-3-{[4-(1H-1,2,3-triazol-1-ylmethyl)-1-naphthoyl]amino}pyridine-2-carboxylate (500 mg, 1.2 mmol, from Example 1D and 1-(tetrahydro-2H-pyran-4-yl)methanamine (395 mg, 3.42 mmol) in DMF (3 ml) was heated at 80° C. for 3 h. The solvent was evaporated under reduced pressure and the residue was purified by preparative HPLC using acetonitrile and ammonium acetate buffer (20:80 to 90:10) as eluent to give 473 mg (79%) of 6-methoxy-N-(tetrahydro-2H-pyran-4-ylmethyl)-3-{[4-(1H-1,2,3-triazol-1-ylmethyl)-1-naphthoyl]amino}pyridine-2-carboxamide.

¹H-NMR (300 MHz, CDCl₃): 1.30-1.41 (m, 2H), 1.60-1.70 (m, 2H), 1.80-1.94 (m, 1H), 3.26-3.43 (m, 4H), 3.96 (s, 3H), 3.96-4.02 (m, 2H), 6.06 (s, 2H), 7.04 (d, J=9.23 Hz, 1H), 7.39 (d, J=0.84 Hz, 1H), 7.43 (d, J=7.22 Hz, 1H), 7.54-7.64 (m, 2H), 7.69 (d, J=0.84 Hz, 1H), 7.85 (d, J=7.21 Hz, 1H), 7.96-8.04 (m, 1H), 8.27 (t, J=6.21 Hz, 1H), 8.51-8.59 (m, 1H), 9.33 (d, J=9.07 Hz, 1H), 12.55 (s, 1H). MS (ESI) (M+H)⁺ 501.12.

### Example 11

### 6-{[(Tetrahydro-2H-pyran-4-ylmethyl)amino]carbonyl}-5-{[4-(1H-1,2,3-triazol-1-ylmethyl)-1-naphthoyl]amino}pyridin-2-yl Acetate

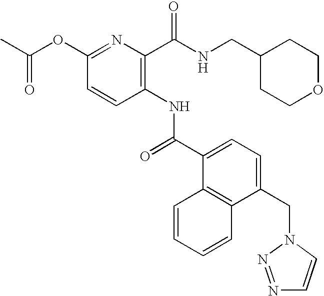

Following the procedure disclosed in Example 1A, using 6-hydroxy-N-(tetrahydro-2H-pyran-4-ylmethyl)-3-{[4-(1H-1,2,3-triazol-1-ylmethyl)-1-naphthoyl]amino}pyridine-2-carboxamide (50 mg, 0.103 mmol), prepared as described in examples 10B-10C, and acetyl chloride (16 mg, 0.21 mmol) provided the title compound after purification by column chromatography on silica gel using CH₂Cl₂/MeOH (100:1.5) as eluent (52 mg, 96%).

¹H NMR (500 MHz, CDCl₃) δ (ppm) 1.32-1.4 (m, 2H), 1.62-1.68 (m, 2H), 1.80-1.90 (m, 1H), 2.39 (s, 3H), 3.29 (t, J=6.6 Hz, 2H), 3.38-3.40 (m, 2H), 3.95-4.01 (m, 2H), 6.08 (s, 2H), 7.32 (d, J=8.9 Hz, 1H), 7.42 (d, J=1 Hz, 1H), 7.44 (d, J=7.0 Hz, 1H), 7.58-7.65 (m, 2H), 7.71 (d, J=1 Hz, 1H), 7.86 (d, J=7.0 Hz, 1H), 8.01-8.06 (m, 1H), 8.21 (t, J=6.6 Hz, 1H), 8.53-8.57 (m, 1H), 9.55 (d, J=8.9 Hz, 1H), 12.8 (s, 1H). MS (ESI) (M+H)⁺ 529.32.

### Example 12

### N-(cyclobutylmethyl)-6-(2-hydroxyethoxy)-3-{[4-(1H-1,2,3-triazol-1-ylmethyl)-1-naphthoyl]amino}pyridine-2-carboxamide

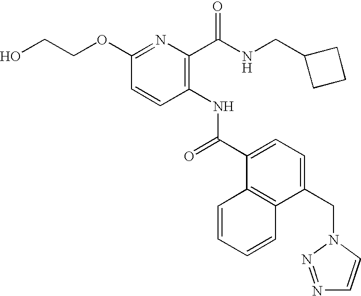

Following the procedure disclosed in Example 1A, using N-(cyclobutylmethyl)-6-hydroxy-3-{[4-(1H-1,2,3-triazol-1-ylmethyl)-1-naphthoyl]amino}pyridine-2-carboxamide (50 mg, 0.11 mmol) prepared in Example 2B, and 2-bromoethanol (41 mg, 0.33 mmol) provided the title compound (27 mg, 49%) and the by-product N-(cyclobutylmethyl)-6-[2-(2-hydroxyethoxy)ethoxy]-3-{[4-(1H-1,2,3-triazol-1-ylmethyl)-1-naphthoyl]amino}pyridine-2-carboxamide (see Example 12A)(1 mg, 2%) after purification by preparative HPLC.

¹H NMR (600 MHz, CDCl₃) δ (ppm) 1.70-1.78 (m, 2H), 1.85-2.0 (m, 2H), 2.05-2.13 (m, 2H), 2.52-2.62 (m, 1H), 3.40 (t, J=6.4 Hz, 2H), 4.0-4.04 (m, 2H), 4.39-4.43 (m, 2H), 6.06 (s, 2H), 7.06 (d, J=9.1 Hz, 1H), 7.40 (s, 1H), 7.46 (d, J=7.2 Hz, 1H), 7.54-7.62 (m, 2H), 7.69 (s, 1H), 7.85 (d, J=7.2 Hz, 1H), 7.99 (d, J=7.6 Hz, 1H), 8.06-8.12 (m, 1H), 8.54 (d, J=8.0 Hz, 1H), 9.34 (d, J=9.1 Hz, 1H), 12.64 (s, 1H). MS (ESI) (M+H)⁺ 501.

### Example 12A

### N-(cyclobutylmethyl)-6-[2-(2-hydroxyethoxy)ethoxy]-3-{[4-(1H-1,2,3-triazol-1-ylmethyl)-1-naphthoyl]amino}pyridine-2-carboxamide

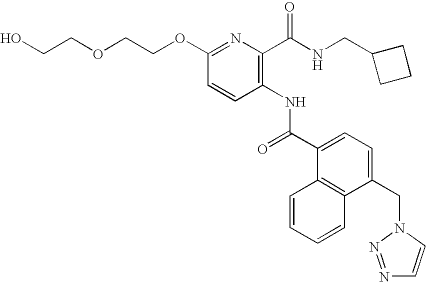

The compound was isolated as a by-product from the synthesis of N-(cyclobutylmethyl)-6-(2-hydroxyethoxy)-3-{[4-(1H-1,2,3-triazol-1-ylmethyl)-1-naphthoyl]amino}pyridine-2-carboxamide (1 mg, 1%) (see Example 12).

¹H NMR (300 MHz, CDCl₃) δ (ppm) 1.70-1.80 (m, 2H), 1.84-1.97 (m, 2H), 2.0-2.16 (m, 2H), 2.52-2.64 (m, 1H), 3.42 (t, J=6.4 Hz, 2H), 3.67-3.72 (m, 2H), 3.77-3.83 (m, 2H), 3.89-3.94 (m, 2H), 4.43-4.49 (m, 2H), 6.06 (s, 2H), 7.07 (d, J=9.2 Hz, 1H), 7.38 (s, 1H), 7.43 (d, J=7.6 Hz, 1H), 7.55-7.65 (m, 2H), 7.67 (s, 1H), 7.85 (d, J=7.4 Hz, 1H), 7.97-8.14 (m, 2H), 8.51-8.58 (m, 1H), 9.35 (d, J=9.1 Hz, 1H), 12.64 (s, 1H). MS (ESI) (M+H)⁺ 545.

### Example 13

### 6-(Benzyloxy)-N-(tetrahydro-2H-pyran-4-ylmethyl)-3-{[4-(1H-1,2,3-triazol-1-ylmethyl)-1-naphthoyl]amino}pyridine-2-carboxamide

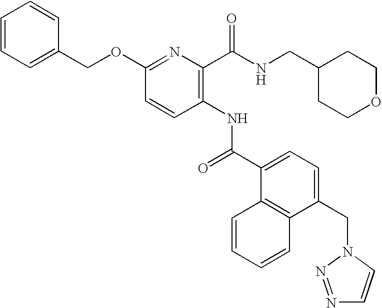

Following the procedure disclosed in Example 1A, using 6-hydroxy-N-(tetrahydro-2H-pyran-4-ylmethyl)-3-{[4-(1H-1,2,3-triazol-1-ylmethyl)-1-naphthoyl]amino}pyridine-2-carboxamide (50 mg, 0.103 mmol), prepared as described in examples 10B-10C, and benzyl bromide (35 mg, 0.21 mmol) provided the title compound (12 mg, 20%) and the by-product 3-benzyl-1-[(4-{[(6-(benzyloxy)-2-{[(tetrahydro-2H-pyran-4-ylmethyl)amino]carbonyl}pyridin-3-yl)amino]carbonyl}-1-naphthyl)methyl]-1H-1,2,3-triazol-3-ium (52 mg, 68%) after purification by column chromatography on silica gel using CH₂Cl₂/MeOH (100:2.5 and 100:15) as eluent.

¹H NMR (600 MHz, CDCl₃) δ (ppm) 1.22-1.34 (m, 2H), 1.51-1.58 (m, 2H), 1.70-1.80 (m, 1H), 3.21 (t, J=6.7 Hz, 2H), 3.29-3.36 (m, 2H), 3.91-3.97 (m, 2H), 5.35 (s, 2H), 6.04 (s, 2H), 7.10 (d, J=9.1 Hz, 1H), 7.28-7.33 (m, 1H), 7.35-7.42 (m, 6H), 7.53-7.60 (m, 2H), 7.66 (s, 1H), 7.81 (d, J=7.3 Hz, 1H), 7.95-8.02 (m, 2H), 8.52 (d, J=8.5 Hz, 1H), 9.33 (d, J=9.1 Hz, 1H), 12.50 (s, 1H). MS (ESI) (M+H)⁺ 577.16

### Example 13A

### 3-Benzyl-1-[(4-{[(6-(benzyloxy)-2-{[(tetrahydro-2H-pyran-4-ylmethyl)amino]carbonyl}pyridin-3-yl)amino]carbonyl}-1-naphthyl)methyl]-1H-1,2,3-triazol-3-ium

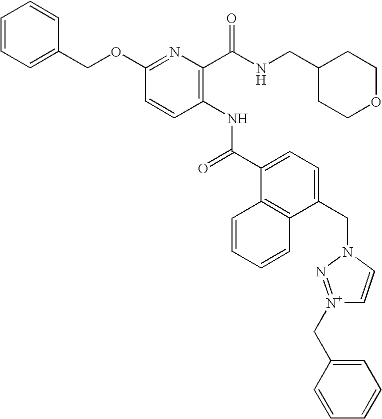

The compound was isolated as a by-product from the synthesis (see Example 13) of 6-(Benzyloxy)-N-(tetrahydro-2H-pyran-4-ylmethyl)-3-{[4-(1H-1,2,3-triazol-1-ylmethyl)-1-naphthoyl]amino}pyridine-2-carboxamide (52 mg, 68%).

¹H NMR (500 MHz, CDCl₃) δ (ppm) 1.20-1.30 (m, 2H), 1.48-1.54 (m, 2H), 1.69-1.77 (m, 1H), 3.17 (t, J=6.7 Hz, 2H), 3.27-3.33 (m, 2H), 3.88-3.94 (m, 2H), 5.34 (s, 2H), 5.91 (s, 2H), 6.53 (s, 2H), 7.08 (d, J=9.2 Hz, 1H), 7.26-7.41 (m, 8H), 7.46-7.49 (m, 2H), 7.54-7.59 (m, 2H), 7.82 (d, J=7.2 Hz, 1H), 7.97 (t, J=6.4 Hz, 1H), 8.04 (d, J=7.3 Hz, 1H), 8.15 (d, J=8.2 Hz, 1H), 8.47 (d, J=7.6 Hz, 1H), 9.28 (d, J=9.2 Hz, 1H), 9.37 (s, 1H), 9.50 (s, 1H), 12.52 (s, 1H).

### Example 14

### N-(cyclobutylmethyl)-6-(pyridin-2-ylmethoxy)-3-{[4-(1H-1,2,3-triazol-1-ylmethyl)-1-naphthoyl]amino}pyridine-2-carboxamide

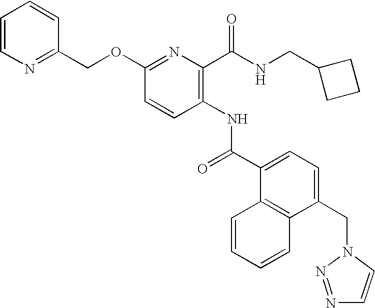

Following the procedure disclosed in Example 1A, using N-(cyclobutylmethyl)-6-hydroxy-3-{[4-(1H-1,2,3-triazol-1-ylmethyl)-1-naphthoyl]amino}pyridine-2-carboxamide (50 mg, 0.11 mmol) prepared in Example 2B, and 2-bromomethyl-pyridine hydrobromide (70 mg, 0.28 mmol) provided the title compound after purification by preparative HPLC (31 mg, 52%).

¹H NMR (300 MHz, CDCl₃) δ (ppm) 1.50-1.79 (m, 2H), 1.81-1.98 (m, 2H), 2.0-2.14 (m, 2H), 2.47-2.62 (m, 1H), 3.35 (t, J=6.9 Hz, 2H), 5.49 (s, 2H), 6.05 (s, 2H), 7.15 (d, J=9.1 Hz, 1H), 7.21-7.28 (m, 1H), 7.37 (s, 1H), 7.39-7.50 (m, 2H), 7.52-7.63 (m, 2H), 7.65-7.76 (m, 2H), 7.82 (d, J=7.2 Hz, 1H), 7.95-8.03 (m, 1H), 8.13 (t, J=6.0 Hz, 1H), 8.50-8.64 (m, 2H), 9.35 (d, J=9.1 Hz, 1H), 12.61 (s, 1H). MS (ESI) (M+H)⁺ 548.21.

### Examples 15 & 16

### N-(cyclobutylmethyl)-3-[(4-{[5-(methoxymethyl)-1H-1,2,3-triazol-1-yl]methyl}-1-naphthoyl)amino]pyridine-2-carboxamide and N-(cyclobutylmethyl)-3-[(4-{[4-(methoxymethyl)-1H-1,2,3-triazol-1-yl]methyl}-1-naphthoyl)amino]pyridine-2-carboxamide

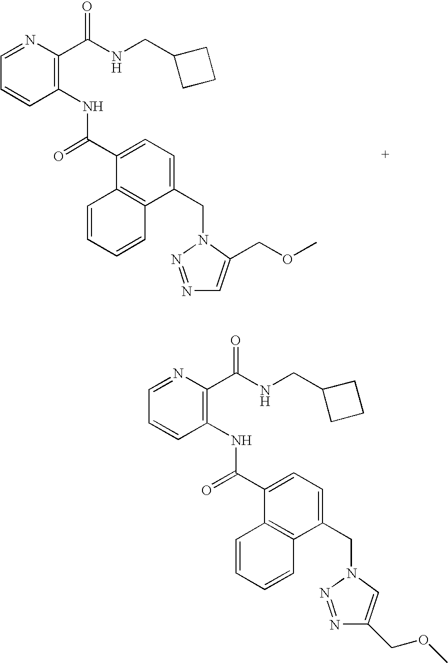

### Examples 15&16:A

### N-(cyclobutylmethyl)-3-[(4-([5-(methoxymethyl)-1H-1,2,3-triazol-1-yl]methyl)-1-naphthoyl)amino]pyridine-2-carboxamide and N-(cyclobutylmethyl)-3-[(4-{[4-(methoxymethyl)-1H-1,2,3-triazol-1-yl]methyl}-1-naphthoyl)amino]pyridine-2-carboxamide

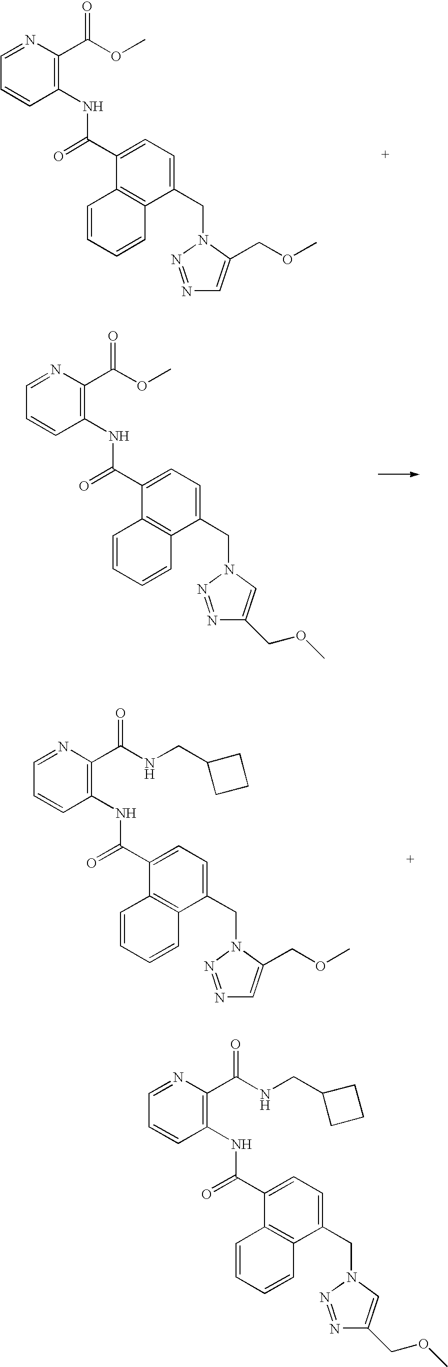

A solution of 1-cyclobutylmethanamine (91 mg, 1.06 mmol) in dry DMF (1 mL) was added to a solution containing a mixture of methyl 3-[(4-{[5-(methoxymethyl)-1H-1,2,3-triazol-1-yl]methyl}-1-naphthoyl)amino]pyridine-2-carboxylate and methyl 3-[(4-{[4-(methoxymethyl)-1H-1,2,3-triazol-1-yl]methyl}-1-naphthoyl)amino]pyridine-2-carboxylate (153 mg), which is obtained from Example 15 & 16:B, and dry DMF (2 mL). The reaction mixture was stirred at 80° C. overnight (18 h) and was then cooled to room temperature and concentrated on a rotary evaporator. Flash column chromatography (toluene/EtOH 15:1 and MTBE) of the residue gave N-(cyclobutylmethyl)-3-[(4-{[5-(methoxymethyl)-1H-1,2,3-triazol-1-yl]methyl}-1-naphthoyl)amino]pyridine-2-carboxamide (46 mg, ˜18% from crude azide):

¹H NMR (400 MHz, CDCl₃) δ (ppm) 1.72-1.77 (m, 2H), 1.83-1.93 (m, 2H), 2.03-2.11 (m, 2H), 2.49-2.61 (m, J=7.6 Hz, 1H), 3.27 (s, 3H), 3.37 (d, J=6.2 Hz, 1H), 3.39 (d, J=7.0 Hz, 1H), 4.28 (s, 2H), 6.12 (s, 2H), 7.10 (d, J=7.3 Hz, 1H), 7.50 (dd, J=4.4, 8.7 Hz, 1H), 7.57-7.63 (m, 2H), 7.66 (s, 1H), 7.79 (d, J=7.3 Hz, 1H), 8.17-8.22 (m, 1H), 8.26 (dd, J=1.4, 4.4 Hz, 1H), 8.35-8.44 (m, 1H), 8.53-8.57 (m, 1H), 9.35 (dd, J=1.4, 8.5 Hz, 1H), 12.87 (br s, 1H); MS (ESI) (M+H)⁺ 485.2;
And N-(cyclobutylmethyl)-3-[(4-{[4-(methoxymethyl)-1H-1,2,3-triazol-1-yl]methyl}-1-naphthoyl)amino]pyridine-2-carboxamide (42 mg, ˜17% from crude azide):

¹H NMR (400 MHz, CDCl₃) δ (ppm) 1.73-1.77 (m, 2H), 1.84-1.96 (m, 2H), 2.04-2.12 (m, 2H), 2.50-2.62 (m, J==7.7 Hz, 1H), 3.34 (s, 3H), 3.39 (d, J=6.8 Hz, 1H), 3.41 (d, J=6.5 Hz, 1H), 4.50 (s, 2H), 6.00 (s, 2H), 7.35 (s, 1H), 7.45 (d, J=7.3 Hz), 7.51 (dd, J=4.5, 8.6 Hz, 1H), 7.54-7.61 (m, 2H), 7.85 (d, J=7.3 Hz, 1H), 7.98-8.02 (m, 1H), 8.27 (dd, J=1.4, 4.4 Hz, 1H), 8.40-8.45 (m, 1H), 8.52-8.56 (m, 1H), 9.37 (dd, J=1.3, 8.6 Hz, 1H), 12.91 (br s, 1H); MS (ESI) (M+H)⁺ 485.2.

### Examples 15&16:B

### Methyl 3-[(4-{[5-(methoxymethyl)-1H-1,2,3-triazol-1-yl]methyl}-1-naphthoyl)amino]pyridine-2-carboxylate and Methyl 3-[(4-{[4-(methoxymethyl)-1H-1,2,3-triazol-1-yl]methyl}-1-naphthoyl)amino]pyridine-2-carboxylate

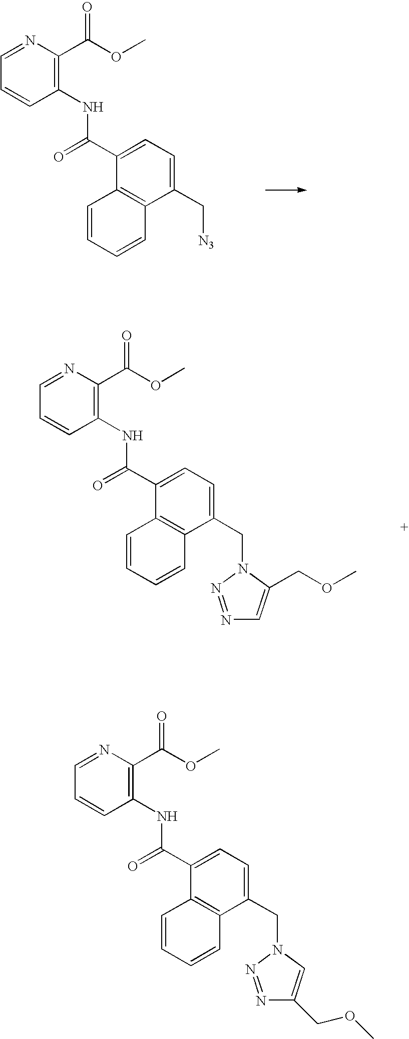

Methyl propargyl ether (0.234 mL, 2.77 mmol) was added to a suspension containing crude methyl 3-{[4-(azidomethyl)-1-naphthoyl]amino}pyridine-2-carboxylate (200 mg, 0.553 mmol), which was obtained from 15 & 16: C, and dry toluene (8 mL). The reaction vessel was sealed and stirred at room temperature for 5 min and then at 130° C. overnight (20 h). The reaction mixture was then cooled to room temperature, concentrated on a rotary evaporator, and subjected to flash column chromatography (CH₂Cl₂/MeOH 30:1) to give a mixture of methyl 3-[(4-{[5-(methoxymethyl)-1H-1,2,3-triazol-1-yl]methyl}-1-naphthoyl)amino]pyridine-2-carboxylate and methyl 3-[(4-{[4-(methoxymethyl)-1H-1,2,3-triazol-1-yl]methyl}-1-naphthoyl)amino]pyridine-2-carboxylate (164 mg): Both compounds had MS (ESI) (M+H)⁺ 432.1.

### Examples 15&16:C

### Methyl 3-{[4-(azidomethyl)-1-naphthoyl]amino}pyridine-2-carboxylate

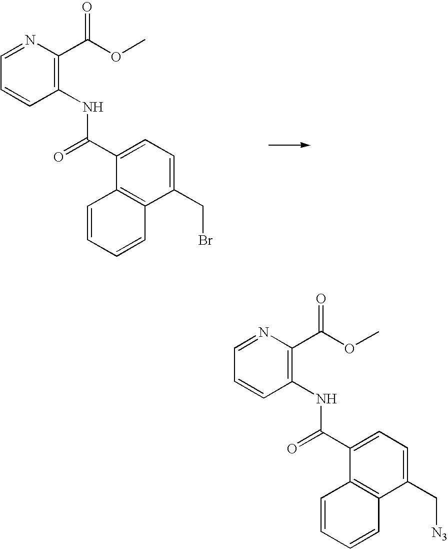

Sodium azide (243 mg, 3.74) was added to a solution of crude methyl 3-{[4-(bromomethyl)-1-naphthoyl]amino}pyridine-2-carboxylate (1.27 g, 3.12 mmol), obtained by using the procedure disclosed in Example 1D isolating the bromo-intermediate by extractive aqueous work up, and dry DMF (15 mL). The reaction mixture was stirred at room temperature for 3 h and then the mixture was partitioned between toluene and water. The aqueous phase was extracted three times with toluene and the combined organic phases were dried (Na₂SO₄) and concentrated on a rotary evaporator. Flash column chromatography (CH₂Cl₂/Et₂O 20:1) of the residue gave crude methyl 3-{[4-(azidomethyl)-1-naphthoyl]amino}pyridine-2-carboxylate (932 mg, ˜83%): MS (ESI) (M+H)⁺ 362.1.

### Examples 17 & 18

### N-(cyclobutylmethyl)-3-[(4-{[5-(1-hydroxyethyl)-1H-1,2,3-triazol-1-yl]methyl}-1-naphthoyl)amino]pyridine-2-carboxamide and N-(cyclobutylmethyl)-3-[(4-{[4-(1-hydroxyethyl)-1H-1,2,3-triazol-1-yl]methyl}-1-naphthoyl)amino]pyridine-2-carboxamide

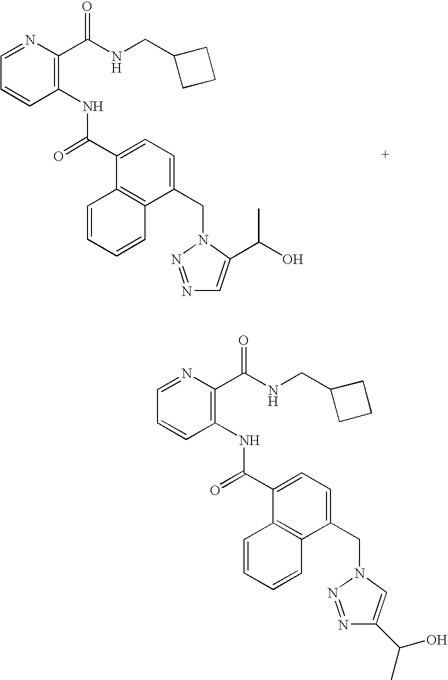

Using procedures analogous to Examples 15&16:A-B, using 3-butyn-2-ol (0.217 mL, 2.77 mmol) and crude methyl 3-{[4-(azidomethyl)-1-naphthoyl]amino}pyridine-2-carboxylate (200 mg, 0.553 mmol), which was obtained from Example 15&16:C, provided:
N-(cyclobutylmethyl)-3-[(4-{[5-(1-hydroxyethyl)-1H-1,2,3-triazol-1-yl]methyl}-1-naphthoyl)amino]pyridine-2-carboxamide (58 mg, 22% from crude azide):

¹H NMR (400 MHz, CDCl₃) δ (ppm) 1.53 (d, J=7.0 Hz, 3H), 1.67-1.76 (m, 2H), 1.82-1.94 (m, 2H), 1.97 (d, J=6.8 Hz, 1H), 2.02-2.12 (m, 2H), 2.49-2.60 (m, 1H), 3.37 (d, J=6.4 Hz, 1H), 3.39 (d, J=6.6 Hz, 1H), 4.70-4.76 (m, 1H), 6.15 (d, J=15.9 Hz, 1H), 6.22 (d, J=15.9 Hz, 1H), 7.05 (d, J=7.4 Hz, 1H), 7.49 (dd, J=4.4, 8.7 Hz, 1H), 7.58-7.63 (m, 3H), 7.77 (d, J=7.4 Hz, 1H), 8.17-8.21 (m, 1H), 8.26 (dd, J=1.4, 4.4 Hz, 1H), 8.38-8.43 (m, 1H), 8.52-8.56 (m, 1H), 9.34 (dd, J=1.2, 8.7 Hz, 1H), 12.85 (s, 1H); MS (ESI) (M+H)⁺ 485.2;
And N-(cyclobutylmethyl)-3-[(4-{[4-(1-hydroxyethyl)-1H-1,2,3-triazol-1-yl]methyl}-1-naphthoyl)amino]pyridine-2-carboxamide (75 mg, 28% from crude azide):

¹H NMR (400 MHz, CDCl₃) δ (ppm) 1.51 (d, J=6.4 Hz, 3H), 1.65-1.76 (m, 2H), 1.81-1.93 (m, 2H), 2.01-2.12 (m, 2H), 2.22 (d, J=4.6 Hz, 1H), 2.50-2.62 (m, 1H), 3.39 (d, J=6.4 Hz, 1H), 3.41 (d, J=6.8 Hz, 1H), 4.97-5.04 (m, 1H), 5.99 (s, 2H), 7.28 (s, 1H), 7.45 (d, J=7.3 Hz, 1H), 7.51 (dd, J=4.4, 8.5 Hz, 1H), 7.55-7.61 (m, 2H), 7.85 (d, J=7.3 Hz, 1H), 8.00-8.04 (m, 1H), 8.27 (dd, J=1.2, 4.4 Hz, 1H), 8.39-8.45 (m, 1H), 8.52-8.56 (m, 1H), 9.37 (dd, J=1.1, 8.5 Hz, 1H), 12.91 (br s, 1H); MS (ESI) (M+H)⁺ 485.2.

### Examples 19 & 20

### 3-[(4-{[5-(aminocarbonyl)-1H-1,2,3-triazol-1-yl]methyl}-1-naphthoyl)amino]-N-(cyclobutylmethyl)pyridine-2-carboxamide and 3-[(4-{[4-(aminocarbonyl)-1H-1,2,3-triazol-1-yl]methyl}-1-naphthoyl)amino]-N-(cyclobutylmethyl)pyridine-2-carboxamide

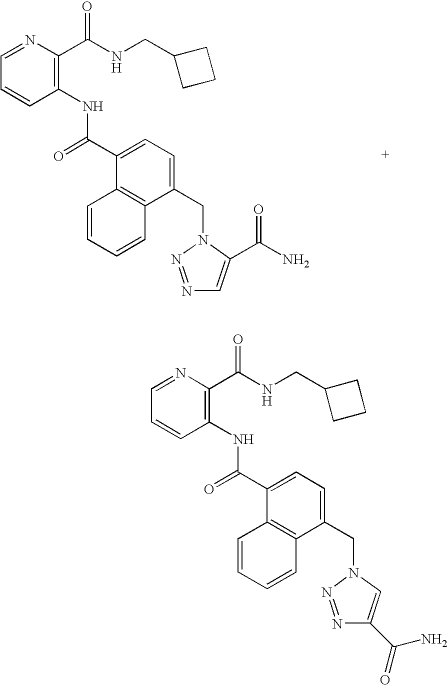

Using procedures analogous to in Examples 15&16:A-B, using propiolamide (191 mg, 2.77 mmol) and crude methyl 3-{[4-(azidomethyl)-1-naphthoyl]amino}pyridine-2-carboxylate (200 mg, 0.553 mmol), which was obtained from Example 15&16:C, provided:
3-[(4-{[5-(aminocarbonyl)-1H-1,2,3-triazol-1-yl]methyl}-1-naphthoyl)amino]-N-(cyclobutylmethyl)pyridine-2-carboxamide (32 mg, 12% from crude azide):

¹H NMR (400 MHz, CDCl₃) δ (ppm) 1.66-1.75 (m, 2H), 1.82-1.93 (m, 2H), 2.02-2.11 (m, 2H), 2.49-2.60 (m, 1H), 3.36 (d, J=6.2 Hz, 1H), 3.38 (d, J=7.0 Hz, 1H), 5.65 (br s, 1H), 5.90 (br s, 1H), 6.45 (s, 2H), 7.22 (d, J=7.6 Hz, 1H), 7.49 (dd, J=4.5, 8.6 Hz, 1H), 7.55-7.64 (m, 2H), 7.76 (d, J=7.3 Hz, 1H), 7.97 (s, 1H), 8.25 (dd, J=1.5, 4.5 Hz, 1H), 8.31-8.35 (m, 1H), 8.38-8.41 (m, 1H), 8.50-8.53 (m, 1H), 9.35 (dd, J=1.4, 8.7 Hz, 1H), 12.81 (s, 1H); MS (ESI) (M+H)⁺ 484.1;
And 3-[(4-{[4-(aminocarbonyl)-1H-1,2,3-triazol-1-yl]methyl}-1-naphthoyl)amino]-N-(cyclobutylmethyl)pyridine-2-carboxamide (32 mg, 12% from crude azide):

¹H NMR (400 MHz, CDCl₃) δ (ppm) 1.68-1.77 (m, 2H), 1.83-1.94 (m, 2H), 2.02-2.12 (m, 2H), 2.50-2.61 (m, 1H), 3.39 (d, J=6.5, 1H), 3.41 (d, J=6.8 Hz, 1H), 5.48 (br s, 1H), 6.04 (s, 2H), 6.97 (br s, 1H), 7.48 (d, J=7.6 Hz, 1H), 7.51 (dd, J=4.6, 8.7 Hz, 1H), 7.56-7.62 (m, 2H), 7.87 (d, J=7.3 Hz, 1H), 7.91 (s, 1H), 7.95-8.00 (m, 1H), 8.27 (dd, J=1.4, 4.4 Hz, 1H), 8.38-8.44 (m, 1H), 8.55-8.59 (m, 1H), 9.36 (dd, J=1.4, 8.5 Hz, 1H), 12.96 (s, 1H); MS (ESI) (M+H)⁺ 484.1.

### Example 21

### 6-(Aminomethyl)-N-(cyclobutylmethyl)-3-{[4-(1H-1,2,3-triazol-1-ylmethyl)-1-naphthoyl]amino}pyridine-2-carboxamide

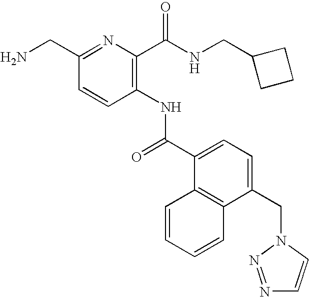

### Example 21A

### 6-(Aminomethyl)-N-(cyclobutylmethyl)-3-{[4-(1H-1,2,3-triazol-1-ylmethyl)-1-naphthoyl]amino}pyridine-2-carboxamide

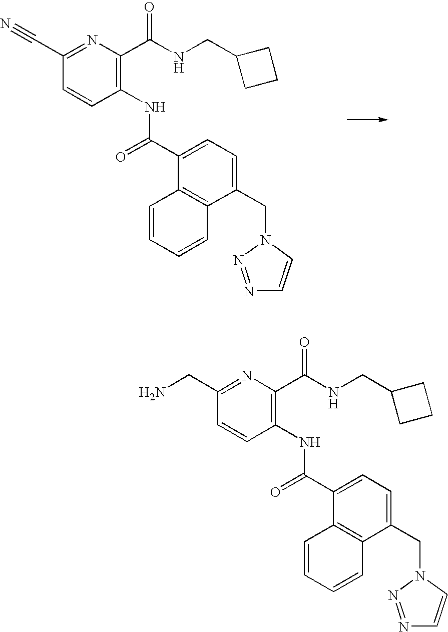

6-Cyano-N-(cyclobutylmethyl)-3-{[4-(1H-1,2,3-triazol-1-ylmethyl)-1-naphthoyl]amino}pyridine-2-carboxamide, obtained from 21B, was hydrogenated in acetic acid (20 ml) catalyzed by 10% Pd/C (70 mg) for 3 h at room temperature. The reaction mixture was filtered over celite and the solvent evaporated under reduced pressure. The residue was purified by column chromatography on silica gel using CH₂Cl₂/MeOH (100:3) and CH₂Cl₂/MeOH/CH₃COOH (100:15:05) as eluent to give the title compound (43 mg, 55%) and N-(cyclobutylmethyl)-6-(hydroxymethyl)-3-{[4-(1H-1,2,3-triazol-1-ylmethyl)-1-naphthoyl]amino}pyridine-2-carboxamide (2 mg, 3%).

¹H NMR (300 MHz, CD₃OD) δ (ppm) 1.70-1.96 (m, 4H), 2.0-2.13 (m, 2H), 2.55-2.68 (m, 1H), 3.38 (d, J=7.18 Hz, 2H), 3.98 (s, 2H), 6.21 (s, 2H), 7.48 (d, J=7.4 Hz, 1H), 7.58 (d, J=8.7 Hz, 1H), 7.60-7.69 (m, 2H), 7.74 (d, J=1 Hz, 1H), 7.89 (d, J=7.2 Hz, 1H), 7.96 (d, J=1 Hz, 1H), 8.20-8.27 (m, 1H), 8.44-8.50 (m, 1H), 9.25 (d, J=8.6 Hz, 1H). MS (ESI) (M+H)⁺ 470.13.

### Example 21B

### 6-Cyano-N-(cyclobutylmethyl)-3-{[4-(1H-1,2,3-triazol-1-ylmethyl)-1-naphthoyl]amino}pyridine-2-carboxamide

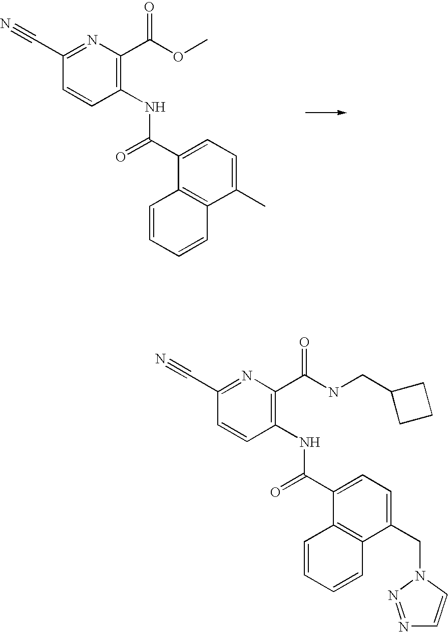

6-Cyano-N-(cyclobutylmethyl)-3-{[4-(1H-1,2,3-triazol-1-ylmethyl)-1-naphthoyl]amino}pyridine-2-carboxamide was formed in a 94% yield (362 mg) following the procedure described in Example 1D and 1C (using cyclobutane methylamine, obtained from 21C).

¹H NMR (500 MHz, CDCl₃) δ (ppm) 1.72-1.82 (m, 2H), 1.89-2.02 (m, 2H), 2.10-2.19 (m, 2H), 2.57-2.68 (m, 1H), 3.47 (t, J=6.6 Hz, 2H), 6.11 (s, 2H), 7.44-7.47 (m, 2H), 7.62-7.69 (m, 2H), 7.74 (s, 1H), 7.89-7.93 (m, 2H), 8.07 (d, J=7.5 Hz, 1H), 8.20-8.28 (m, 1H), 8.58 (d, J=7.0 Hz, 1H), 9.58 (d, J=8.92 Hz, 1H), 13.25 (s, 1H). MS (ESI) (M+H)⁺ 466.05.

### Example 21C

### Methyl 6-cyano-3-[(4-methyl-1-naphthoyl)amino]pyridine-2-carboxylate

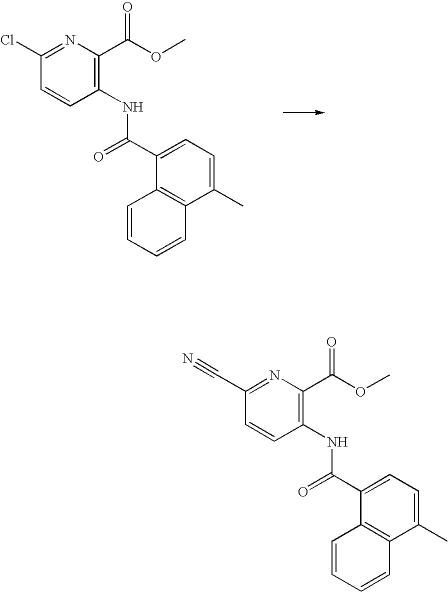

A mixture of methyl 6-chloro-3-[(4-methyl-1-naphthoyl)amino]pyridine-2-carboxylate prepared in Example 21D (500 mg, 1.41 mmol), KCN (75 mg, 1.15 mmol), Pd(AcO)₂ (63 mg, 0.28 mmol), 1,5-bis(diphenylphosphino)pentane (248 mg, 0.56 mmol) and TMEDA (328 mg, 2.82 mmol) in anhydrous toluene (20 ml) was heated in the microwave at 160° C. for 40 min. The reaction mixture was diluted with dichloromethane and then filtered. The solvent was concentrated on a rotary evaporator and the residue was suspended in methanol and heated to reflux, and then allowed to reach room temperature. The crystals was filtered, washed with methanol, dried in vacuo, and purified by column chromatography on silica gel using dichloromethane as eluent to give the title compound (250 mg, 51%). MS (ESI) (M+H)⁺ 346.05.

### Example 21D

### Methyl 6-chloro-3-[(4-methyl-1-naphthoyl)amino]pyridine-2-carboxylate

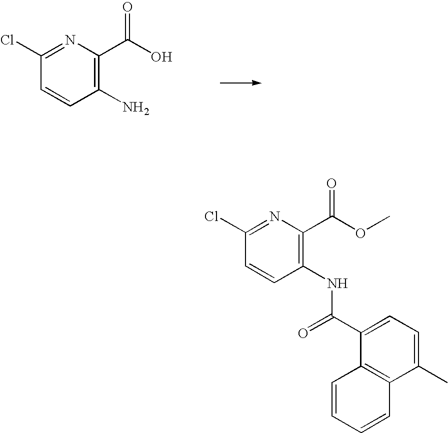

3-Amino-6-chloropyridine-2-carboxylic acid, obtained by the procedure of Goldberg et al. [Besly; Goldberg; JCSOA9; J. Chem. Soc.; 2448, 2455] from 6-chloro-2-methylpyridin-3-amine, was transformed into methyl 6-chloro-3-[(4-methyl-1-naphthoyl)amino]pyridine-2-carboxylate in a 66% yield (11.42 g) using the procedure described in Example 1E. MS (ESI) (M+H)⁺ 355.13.

### Example 21E

### N-(cyclobutylmethyl)-6-(hydroxymethyl)-3-{[4-(1H-1,2,3-triazol-1-ylmethyl)-1-naphthoyl]amino}pyridine-2-carboxamide

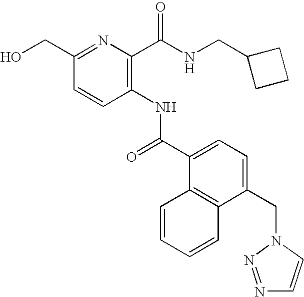

The compound was isolated as a by-product from the synthesis of 6-(Aminomethyl)-N-(cyclobutylmethyl)-3-{[4-(1H-1,2,3-triazol-1-ylmethyl)-1-naphthoyl]amino}pyridine-2-carboxamide (2 mg, 3%) (see Example 21A).

¹H NMR (300 MHz, CDCl₃) δ (ppm) 1.58-1.81 (m, 2H), 1.82-2.0 (m, 2H), 2.02-2.20 (m, 2H), 2.51-2.68 (m, 1H), 3.40-3.48 (m, 2H), 4.81 (s, 2H), 6.07 (s, 2H), 7.41 (s, 1H), 7.44 (d, J=7.4 Hz, 1H), 7.55-7.65 (m, 3H), 7.70 (s, 1H), 7.87 (d, J=7.2 Hz, 1H), 7.98-8.05 (m, 1H), 8.27-8.37 (m, 1H), 8.53-8.60 (m, 1H), 9.42 (d, J=8.7 Hz, 1H), 12.89 (s, 1H). MS (ESI) (M+H)⁺ 471.09.

### Example 22

### N-(cyclobutylmethyl)-6-{[(methylsulfonyl)amino]methyl}-3-{[4-(1H-1,2,3-triazol-1-ylmethyl)-1-naphthoyl]amino}pyridine-2-carboxamide

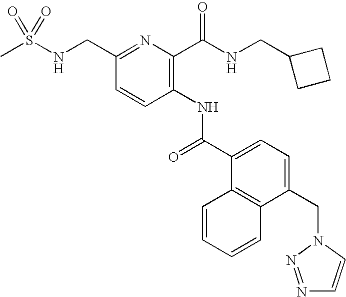

To a solution of 6-(aminomethyl)-N-(cyclobutylmethyl)-3-{[4-(1H-1,2,3-triazol-1-ylmethyl)-1-naphthoyl]amino}pyridine-2-carboxamide (40 mg, 0.084 mmol), which was obtained from Example 21A, and anhydrous dichloromethane (3 ml) was added triethylamine (17 mg, 0.17 mmol) and methanesulphonyl chloride (20 mg, 0.17 mmol) at 0° C. under nitrogen. The reaction mixture was stirred for 35 min at room temperature. After the addition of CH₂Cl₂, the reaction mixture was washed with NaHCO₃ (aq, sat), dried and evaporated under reduced pressure. The residue was purified by column chromatography on silica gel using CH₂Cl₂/MeOH (100:5) as eluent to give the title compound (45 mg, 97%).

¹H NMR (300 MHz, CDCl₃) δ (ppm) 1.69-1.80 (m, 2H), 1.83-1.98 (m, 2H), 2.02-2.18 (m, 2H), 2.51-2.68 (m, 1H), 2.98 (s, 3H), 3.38-3.46 (m, 2H), 4.49 (s, 2H), 5.45 (br s, 1H), 6.06 (s, 2H), 7.41 (s, 1H), 7.43 (d, J=7.0 Hz, 1H), 7.52 (d, J=8.8 Hz, 1H), 7.55-7.64 (m, 2H), 7.70 (s, 1H), 7.86 (d, J=7.2 Hz, 1H), 7.97-8.04 (m, 1H), 8.50-8.62 (m, 2H), 9.39 (d, J=8.7 Hz, 1H), 12.94 (s, 1H). MS (ESI) (M+H)⁺ 548.

### Example 23

### Methyl-6-{[(cyclobutylmethyl)amino]carbonyl}-5-{[4-(1H-1,2,3-triazol-1-yl methyl)-1-naphthoyl]amino}pyridine-2-carboxylate

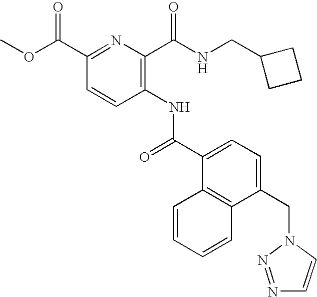

A mixture of 6-cyano-N-(cyclobutylmethyl)-3-{[4-(1H-1,2,3-triazol-1-ylmethyl)-1-naphthoyl]amino}pyridine-2-carboxamide obtained from Example 21 (60 mg, 0.13 mmol) and NaOH (21 mg dissolved in 0.4 ml water) in methanol (3 ml) was heated in the microwave at 80° C. for 7 min. The solution was adjusted to pH 4 with 2 N HCl (aq). The solvents were removed in vacuo. The residue was dissolved in dichloromethane, washed with NaHCO₃ (aq, sat), dried and evaporated under reduced pressure. The residue was purified by column chromatography on silica gel using CH₂Cl₂/MeOH (100:2) as eluent to give the title compound (31 mg, 48%).

¹H NMR (300 MHz, CDCl₃) δ (ppm) 1.63-1.98 (m, 4H), 2.02-2.18 (m, 2H), 2.53-2.63 (m, 1H), 3.38-3.43 (m, 2H), 4.0 (s, 3H), 6.06 (s, 2H), 7.38-7.42 (m, 2H), 7.57-7.63 (m, 2H), 7.72 (s, 1H), 7.9 (d, 1H), 7.96-8.06 (m, 1H), 8.3 (d, 1H), 8.45-8.58 (m, 2H), 9.45 (d, 1H), 13.18 (s, 1H). MS (ESI) (M+H)⁺ 499.

### Example 24

### N²-(cyclobutylmethyl)-3-{[4-(1H-1,2,3-triazol-1-ylmethyl)-1-naphthoyl]amino}pyridine-2,6-dicarboxamide

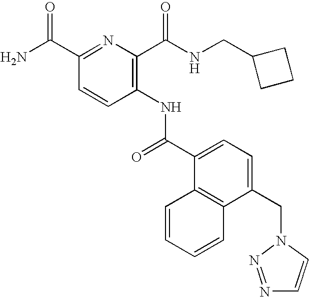

A mixture of 6-cyano-N-(cyclobutylmethyl)-3-{[4-(1H-1,2,3-triazol-1-ylmethyl)-1-naphthoyl]amino}pyridine-2-carboxamide, obtained from Example 21B, (45 mg, 0.097 mmol) and NaOH (37 mg dissolved in 0.8 ml water) and ethanol (2.7 ml) was heated in the microwave at 80° C. for 5 min. The solution was adjusted to pH 4 with 2 N HCl (aq). Ethanol (1 ml) and water (1.5 ml) was added. The formed precipitate was collected, washed with water, ethanol and ether and then dried in vacuo to give the title compound (33 mg, 70%).

¹H NMR (500 MHz, DMSO-d₆) δ (ppm) 1.64-1.73 (m, 2H), 1.74-1.84 (m, 2H), 1.91-2.0 (m, 2H), 2.48-2.58 (m, 1H), 3.32-3.42 (m, 2H), 6.22 (s, 2H), 7.39 (d, J=7.5 Hz, 1H), 7.64-7.74 (m, 3H), 7.78 (s, 1H), 7.91 (d, J=7.5 Hz, 1H), 8.25 (s, 1H), 8.27-8.34 (m, 2H), 8.42 (d, J=8.4 Hz, 1H), 8.80 (s, 1H), 9.34 (d, J=8.4 Hz, 1H), 9.72 (t, J=6.1 Hz, 1H), 13.23 (s, 1H). MS (ESI) (M+H)⁺ 484.08.

### Example 25

### N-(cyclobutylmethyl)-6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxamide

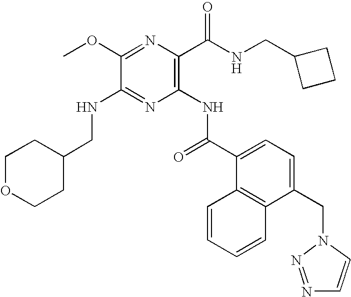

### Example 25A

### N-(cyclobutylmethyl)-6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxamide

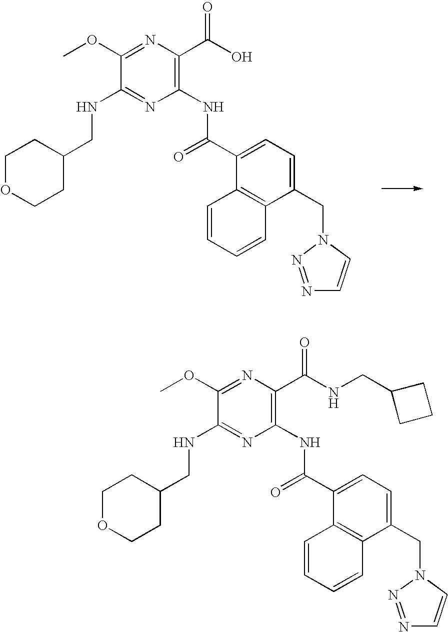

To a mixture of 6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxylic acid prepared in Example 25B (0.034 g, 0.06 mmol, 1 equivalent) and one drop of N,N-dimethylformamide in 6 mL chloroform was added a solution of oxalyl chloride (0.093 g, 0.73 mmol, 11.1 equivalent (eq)) in 2 mL chloroform. The mixture was heated at 90° C. for 3 h until LC/MS showed that the carboxylic acid had completely changed into carbonyl chloride. The solvents were removed in vacuo. To the residue was added a solution of N,N-diisopropylethylamine (0.077 g, 0.59 mmol, 9.1 equivalents) and cyclobutyl methylamine (0.031 g, 0.36 mmol, 5.5 equivalents) in 10 mL anhydrous acetonitrile. The mixture was stirred at room temperature for 5 h. After removal of solvents, the residue was dissolved in 5 mL dimethyl sulfoxide. The resulting solution was purified by reversed-phase preparative HPLC. Freeze drying gave 0.007 g (18%) of the title compound.

¹H NMR (400 MHz, CDCl₃) δ (ppm) 1.22-1.42 (m, 2H), 1.61 (d, J=12.8 Hz, 2H), 1.67-1.80 (m, 3H), 1.83-1.98 (m, 4H), 2.00-2.14 (m, 2H), 2.55 (m, 1H), 3.27-3.43 (m, 4H), 3.97 (m, 2H), 3.98 (s, 3H), 5.59 (br s, 1H), 6.07 (s, 2H), 7.34-7.49 (m, 3H), 7.56 (m, 2H), 7.68 (s, 1H), 7.81 (d, J=7.3 Hz, 1H), 7.97 (m, 1H), 8.55 (m, 1H), 12.29 (s, 1H).

### Example 25B

### 6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxylic Acid

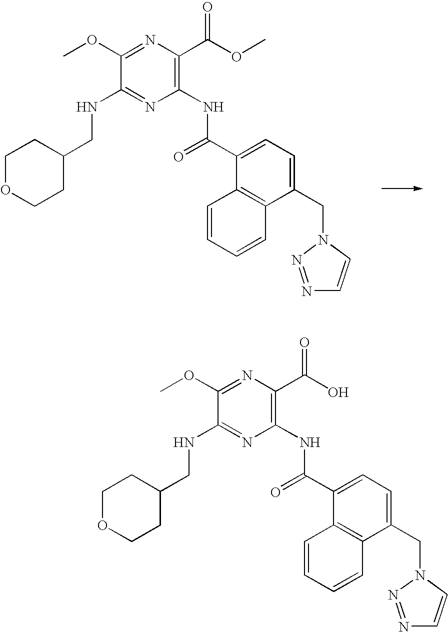

To a mixture of methyl 6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxylate prepared in Example 25C (0.035 g, 0.07 mmol, 1 equivalents) in 3 mL methanol was added an aqueous solution of lithium hydroxide (0.013 mg, 0.54 mmol, 8.2 equivalents) in 2 mL water. The resulting mixture was stirred overnight at room temperature. When LC/MS showed that the hydrolysis was completed, the mixture was neutralized with acetic acid (0.129 g, 2.2 mmol, 30 eq.). The solvents were removed in vacuo. The obtained solid was used directly in the next step without further purification.

### Example 25C

### Methyl-6-methoxy-5-[(tetrahydro-2H-pyran-4-ylmethyl)amino]-3-([4-(1H-1,2,3-triazol-1-ylmethyl)-1-naphthoyl]aminopyrazine-2-carboxylate

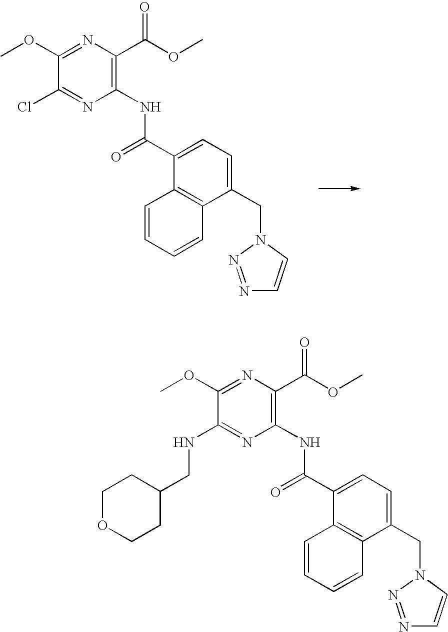

A solution of methyl 5-chloro-6-methoxy-3-{[4-(1H-1,2,3-triazol-1-ylmethyl)-1-naphthoyl]amino}pyrazine-2-carboxylate prepared in Example 25D (0.060 g, 0.13 mmol, 1 eq.) and (tetrahydro-pyran-4-yl)-methylamine (0.064 g, 0.56 mmol, 4.2 eq.) in 5 mL anhydrous dimethylformamide was heated at 120° C. for 3 h until LC/MS showed that the starting material was consumed. The reaction mixture was cooled to room temperature and the solution was purified by reversed-phase preparative HPLC. Freeze drying gave 0.035 g (50%) of title compound.

¹H NMR (400 MHz, DMSO-d₆) δ (ppm) 0.90 (dt, J=9.5, 11.2 Hz, 2H), 1.27 (d, J=12.7 Hz, 2H), 1.64 (m, 1H), 2.75 (br s, 2H), 3.12 (t, J=11.0 Hz, 2H), 3.69-3.79 (m, 2H), 6.17 (s, 2H), 7.37 (d, J=7.3 Hz, 1H), 7.56-7.70 (m, 4H), 7.76 (d, J=0.6 Hz, 1H), 8.20 (d, J=0.6 Hz, 1H), 8.24 (d, J=7.7 Hz, 1H), 8.29 (d, J=7.7 Hz, 1H), 10.84 (s, 1H).

### Example 25D

### Methyl-5-chloro-6-methoxy-3-([4-(1H-1,2,3-triazol-1-ylmethyl)-1-naphthoyl]aminopyrazine-2-carboxylate

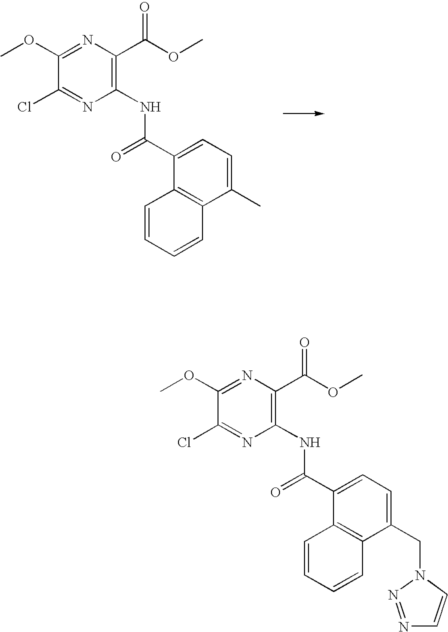

A mixture of methyl 5-chloro-6-methoxy-3-[(4-methyl-1-naphthoyl)amino]pyrazine-2-carboxylate prepared in Example 25E (0.258 g, 0.67 mmol, 1 eq.), N-bromosuccinimide (0.123 g, 0.69 mmol, 1.0 eq.) and benzoyl peroxide (0.018 g, 0.07 mmol, 0.11 eq.) in 100 mL carbon tetrachloride was refluxed for 1 h until LC/MS showed that bromination was complete. The reaction mixture was cooled to 50° C. and 1,2,3-triazole (0.245 g, 3.55 mmol, 5.3 eq.) was added. The resulting mixture was stirred at 80° C. for 2 h until LC/MS is showed that the reaction was complete. The solvents were removed in vacuo. The residue was dissolved in 10 mL dimethyl sulfoxide. The resulting dimethyl sulfoxide solution was purified by reversed-phase preparative HPLC. Freeze drying gave 0.062 g (21%) of title compound.

¹H NMR (400 MHz, DMSO-d₆) δ (ppm) 3.80 (s, 3H), 4.01 (s, 3H), 5.72 (s, 1H), 6.16 (s, 2H), 7.37 (d, J=7.2 Hz, 1H), 7.63 (m, 2H), 7.71 (d, J=7.3 Hz, 1H), 7.73 (s, 1H), 8.18 (s, 1H), 8.26 (m, 2H), 11.41 (br s, 1H).

### Example 25E

### Methyl-5-chloro-6-methoxy-3-[(4-methyl-1-naphthoyl)amino]pyrazine-2-carboxylate

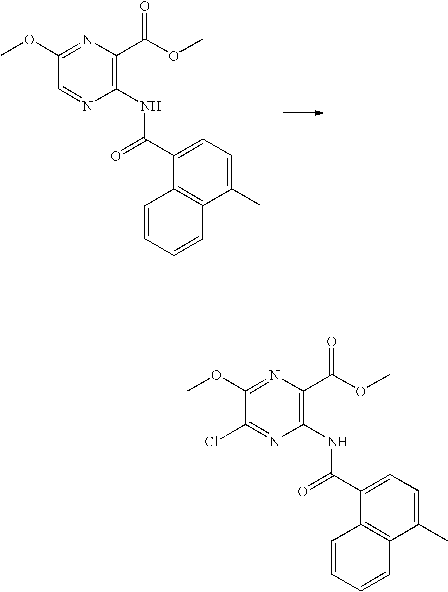

A mixture of methyl 6-methoxy-3-[(4-methyl-1-naphthoyl)amino]pyrazine-2-carboxylate prepared in Example 25F (0.375 g, 1.07 mmol, 1 eq.), N-chlorosuccinimide (0.248 g, 1.86 mmol, 1.7 eq.) in 18 mL anhydrous acetonitrile was heated in the microwave at 120° C. for 10 min. LC/MS showed that the starting material methyl ester was consumed. The resulting solution was purified by reversed-phase preparative HPLC. Freeze drying gave 0.318 g (77%) of the title compound.

¹H NMR (400 MHz, DMSO-d₆) δ (ppm) 3.84 (s, 3H), 3.97 (s, 3H), 6.20 (s, 2H), 7.42 (d, J=7.3 Hz, 1H), 7.67 (m, 2H), 7.74 (d, J=7.3 Hz, 1H), 7.77 (d, J=0.8 Hz, 1H), 8.21 (d, J=0.8 Hz, 1H), 8.30 (m, 2H), 8.43 (s, 1H), 11.29 (br s, 1H).

### Example 25F

### Methyl-6-methoxy-3-[(4-methyl-1-naphthoyl)amino]pyrazine-2-carboxylate

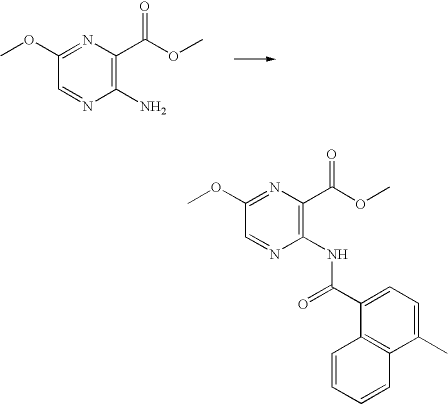

A solution of methyl 3-amino-6-methoxypyrazine-2-carboxylate prepared in Example 25G (1.560 g, 8.52 mmol, 1 eq.), 4-methyl-naphthalene-1-carbonyl chloride (1.830 g, 8.94 mmol, 1.05 eq.), 4-dimethylaminopyridine (0.117 g, 0.95 mmol, 0.11 eq.) and anhydrous pyridine (2.716 g, 34.34 mmol, 4.03 eq.) in 150 mL anhydrous chloroform was stirred at room temperature until there was no starting material left according to LC/MS. The solvents were removed in vacuo. The residue was purified by flash chromatography on silica gel eluted with ethyl acetate/hexane to give the desired product (1.832 g, 61%).

¹H NMR (400 MHz, DMSO-d₆) δ (ppm) 2.72 (s, 3H), 3.83 (s, 3H), 3.98 (s, 3H), 7.48 (d, J=7.4 Hz, 1H), 7.62 (m, 2H), 7.68 (d, J=7.4 Hz, 1H), 8.12 (m, 1H), 8.31 (m, 1H), 8.43 (s, 1H), 11.19 (br s, 1H).

### Example 25G

### Methyl 3-amino-6-methoxypyrazine-2-carboxylate

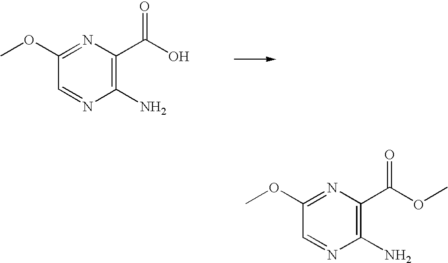

To a solution of 3-amino-6-methoxypyrazine-2-carboxylic acid prepared in Example 25H (1.821 g, 10.77 mmol, 1 eq.) in 40 mL anhydrous 1,4-dioxane, was added 5 mL triethylamine (3.635 g, 35.92 mmol, 3.34 eq.). The mixture was ultrasonicated so that carboxylic acid completely converted into triethylammonium salt. The reaction mixture was cooled in an ice-water bath and ethyl chloroformate (1.725 g, 15.90 mmol, 1.48 eq.) was added. The reaction mixture was stirred at 0° C. for 10 min, and then allowed to reach room temperature. The reaction mixture was continuously stirred for another 20 min until LC/MS showed that there was no starting material remaining. To the mixture was added 20 mL of anhydrous methanol, and the resulting mixture was stirred for 1 h. The solvents were removed in vacuo. The residue was extracted with dichloromethane/water twice. The organic layers were combined, rinsed with water, dried with anhydrous sodium sulfate, and concentrated in vacuo to give a solid. The solid was dissolved in hot methanol and charcoal was added to decolor the product. After filtration, a slightly yellow crystalline solid (1.584 g, 80%) was obtained from the cold methanol solution.

¹H NMR (400 MHz, DMSO-d₆) δ (ppm) 3.77 (s, 3H), 3.79 (s, 3H), 6.92 (s, 2H), 8.09 (s, 1H).

### Example 25H

### 3-Amino-6-methoxypyrazine-2-carboxylic Acid

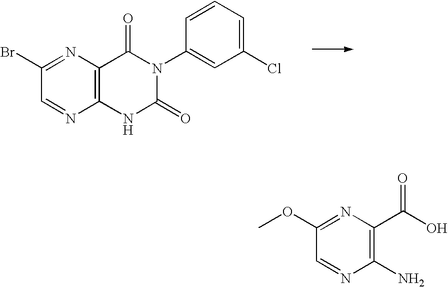

6-Bromo-3-(3-chlorophenyl)pteridine-2,4(1H,3H)-dione prepared in Example 25I (6.750 g, 19.10 mmol, 1 eq.) was mixed with a 30% solution sodium methoxide (3.094 g, 57.27 mmol, 3 eq.) in 84 mL anhydrous methanol. The mixture was heated at 130° C. for 20 h. LC/MS showed that the starting material was consumed. The solvent was removed in vacuo. To the residue, was added an aqueous solution of sodium hydroxide (1.145 g, 28.64 mmol, 1.5 eq. in 150 mL water). The mixture was refluxed for 20 h until the reaction was complete. The reaction mixture was then allowed to reach room temperature and a trace amount of insoluble solid was filtered out. The filtrate was decolored with charcoal and then evaporated to half the volume. The resulting solution was neutralised with 4 N aqueous hydrochloride solution until pH 2-3. After standing at room temperature for 1 h, the solids formed were filtered, rinsed with cold water twice, and dried in vacuo. 2.65 g of solid (82%) was obtained, which was used for methylation without further purification.

¹H NMR (400 MHz, DMSO-d₆) δ (ppm) 3.83 (s, 3H), 8.10 (s, 1H).

### Example 25I

### 6-Bromo-3-(3-chlorophenyl)pteridine-2,4(1H,3H)-dione

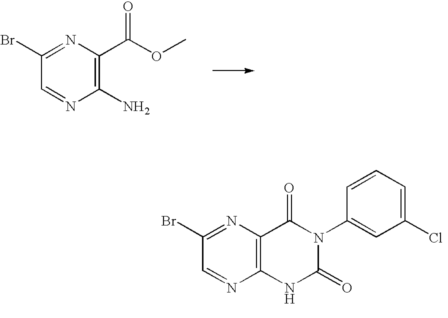

To a solution of methyl 3-amino-6-bromopyrazine-2-carboxylate prepared in Example 25J (11.476 g, 49.45 mmol, 1 eq.) in 60 mL anhydrous pyridine was added 3-chlorophenylisocyanate (8.82 g, 57.43 mmol, 1.2 eq.). The mixture was refluxed at 150° C. for 4 h. The solvent was removed in vacuo. To the residue was added brine. The mixture was extracted with three portions of dichloromethane. The organic phase layers were combined, rinsed with aqueous saturated sodium hydrogencarbonate solution, and evaporated in vacuo. The resulting residue was dissolved in hot ethyl acetate. After standing overnight, the crude product was obtained as a solid. Additional product was extracted from the mother solution. Thus, 8.012 g of the desired product was obtained. The yield from methyl 3-amino-2-pyrazinecarboxylate is 57% in two steps.

¹H NMR (400 MHz, CDCl₃) δ (ppm) 7.19 (dt, J=2.0, 4.5 Hz, 1H), 7.31 (s, 1H), 7.48 (d, J=4.9 Hz, 1H), 8.69 (s, 1H).

### Example 25J

### Methyl 3-amino-6-bromopyrazine-2-carboxylate

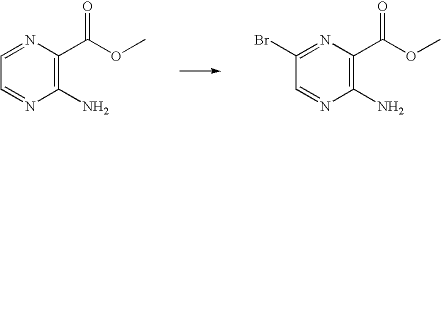

A mixture of methyl 3-aminopyrazine-2-carboxylate (6.30 g, 41.14 mmol, 1 eq.) and N-bromosuccinimide (7.322 g, 41.14 mmol, 1 eq.) in 100 mL acetonitrile was refluxed for 2 h until there was no starting material left according to LC/MS. The solvent was removed in vacuo. To the residue was added isopropanol. After filtration, the crude product was collected as a solid. Additional product could be collected from the mother solution. Thus, 12.136 g of crude product was obtained (127%). The crude product was used directly in the next step without further purification.

¹H NMR (400 MHz, DMSO-d₆) δ (ppm) 3.85 (s, 3H), 7.55 (br s, 2H), 8.42 (s, 1H).

### Example 26

### 6-(2-Morpholin-4-yl-2-oxo-ethoxy)-3-[(4-[1,2,3]triazol-1-ylmethyl-naphthalene-1-carbonyl)-amino]-pyridine-2-carboxylic Acid Cyclobutylmethyl-amide

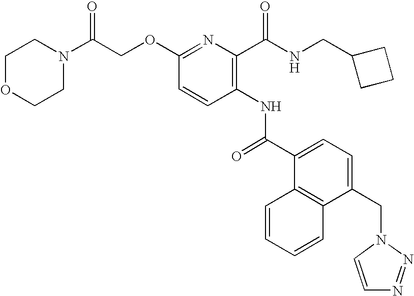

Following the procedure described in Example 4, using [(6-{[(cyclobutylmethyl)amino]carbonyl}-5-{[4-(1H-1,2,3-triazol-1-ylmethyl)-1-naphthoyl]amino}pyridin-2-yl)oxy]acetic acid, which was prepared in Example 3, (44 mg, 0.086 mmol), and morpholine (20 mg, 0.23 mmol) provided the title compound after workup (47 mg, 94%).

¹H NMR (300 MHz, CDCl₃) δ (ppm) 1.70-2.0 (m, 4H), 2.02-2.20 (m, 2H), 2.56-2.72 (m, 1H), 3.37-3.44 (m, 2H), 3.51-3.67 (m, 8H), 3.84 (s, 2H), 6.50 (s, 2H), 7.06 (d, 1H), 7.39 (s, 1H), 7.42 (d, 1H), 7.52-7.64 (m, 2H), 7.68 (s, 1H), 7.84 (d, 1H), 7.95-8.04 (m, 1H), 8.45 (“t”, 1H), 8.50-8.59 (m, 1H), 9.39 (d, 1H), 12.71 (s, 1H). MS (ESI) (M+H)⁺ 584.09.

### Example 27

### 6-(Benzylcarbamoyl-methoxy)-3-[(4-[1,2,3]triazol-1-ylmethyl-naphthalene-1-carbonyl)-amino]-pyridine-2-carboxylic Acid Cyclobutylmethyl-amide

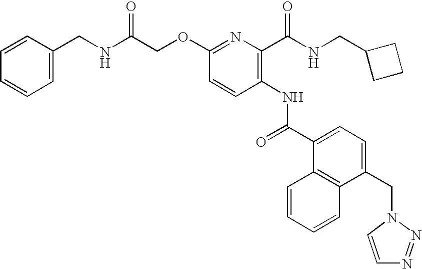

Following the procedure described in Example 4, using [(6-{[(cyclobutylmethyl)amino]carbonyl}-5-{[4-(1H-1,2,3-triazol-1-ylmethyl)-1-naphthoyl]amino}pyridin-2-yl)oxy]acetic acid, which was prepared in Example 3, (40 mg, 0.078 mmol), and benzylamine (25 mg, 0.23 mmol) provided the title compound after purification by column chromatography on silica gel using CH₂Cl₂/MeOH (100:2.25) as eluent (22 mg, 47%).

¹H NMR (500 MHz, CDCl₃) δ (ppm) 1.70-181 (m, 2H), 1.82-2.0 (m, 2H), 2.07-2.16 (m, 2H), 2.56-2.64 (m, 1H), 3.40 (“f”, 2H), 4.52 (d, 2H), 4.82 (s, 2H), 6.10 (s, 2H), 6.60 (t, 1H), 7.11 (d, 1H), 7.20 (d, 2H), 7.25-7.34 (m, 3H), 7.43 (s, 1H), 7.47 (d, 1H), 7.59-7.66 (m, 2H), 7.73 (s, 1H), 7.89 (s, 1H), 8.0-8.06 (m, 2H), 8.58 (d, 1H), 9.42 (d, 1H), 12.74 (s, 1H). MS (ESI) (M+H)⁺ 604.

### Example 28

### {6-(Cyclobutylmethyl-carbamoyl)-5-[(4-[1,2,3]triazol-1-ylmethyl-naphthalene-1-carbonyl)-amino]-pyridin-2-yloxy}-acetic Acid 2,2-dimethyl-propyl Ester

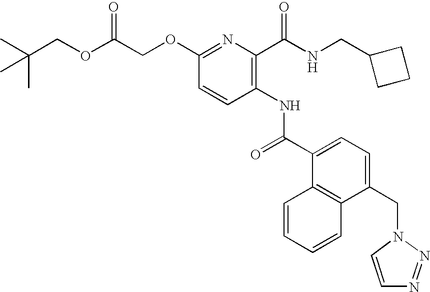

To a mixture of [(6-{[(cyclobutylmethyl)amino]carbonyl}-5-{[4-(1H-1,2,3-triazol-1-ylmethyl)-1-naphthoyl]amino}pyridin-2-yl)oxy]acetic acid, which was prepared in Example 3, (40 mg, 0.078 mmol), in dichloromethane (3 ml) was added triethylamine (32 mg, 0.31 mmol), neopentyl chloroformate (24 mg, 0.16 mmol) and 4-dimethylaminopyridine (6 mg, 0.05 mmol) at 0° C. under nitrogen. The reaction mixture was stirred at 0° C. for 50 min, and then diluted with dichloromethane. The solution was washed with NH₄Cl (aq, sat), dried and evaporated in vacuo. The residue was purified by column chromatography on silica gel using CH₂Cl₂/MeOH (100:2) as eluent to give the title compound (42 mg, 92%).

¹H NMR (500 MHz, CDCl₃) δ (ppm) 0.84 (s, 9H), 1.72-1.84 (m, 2H), 1.86-2.0 (m, 2H), 2.09-2.18 (m, 2H), 2.55-2.65 (m, 1H), 3.41 (“t”, 2H), 3.83 (s, 2H), 4.83 (s, 2H), 6.09 (s, 2H), 7.18 (d, 1H), 7.41 (s, 1H), 7.45 (d, 1H), 7.58-7.65 (m, 2H), 7.71 (s, 1H), 7.87 (d, 1H), 7.90 (t, 1H), 8.01-8.05 (m, 1H), 8.58-8.60 (m, 1H), 9.43 (d, 1H), 12.70 (s, 1H). MS (ESI) (M+H)⁺ 585.08.

### Example 29

### {6-(Cyclobutylmethyl-carbamoyl)-5-[(4-[1,2,3]triazol-1-ylmethyl-naphthalene-1-carbonyl)-amino]-pyridin-2-yloxy}-acetic Acid Isopropyl Ester

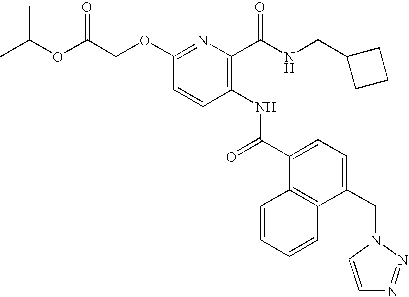

Following the procedure described in Example 28, using [(6-{[(cyclobutylmethyl)amino]carbonyl}-5-{[4-(1H-1,2,3-triazol-1-ylmethyl)-1-naphthoyl]amino}pyridin-2-yl)oxy]acetic acid, which was prepared in Example 3, (40 mg, 0.078 mmol), and isopropyl chloroformate (0.12 ml 1M in toluene, 0.12 mmol) provided the title compound after purification by column chromatography on silica gel using CH₂Cl₂/MeOH (100:2.0) as eluent (41 mg, 94%).

¹H NMR (500 MHz, CDCl₃) δ (ppm) 1.21 (d, 6H), 1.72-1.82 (m, 2H), 1.85-2.02 (m, 2H), 2.10-2.18 (m, 2H), 2.56-2.66 (m, 1H), 3.42 (“t”, 2H), 4.77 (s, 2H), 5.07-5.16 (m, 1H), 6.09 (s, 2H), 7.18 (d, 1H), 7.43 (s, 1H), 7.47 (d, 1H), 7.58-7.66 (m, 2H), 7.73 (s, 1H), 7.88 (d, 1H), 7.95 (t, 1H), 8.0-8.05 (m, 1H), 8.56-8.60 (m, 1H), 9.43 (d, 1H), 12.70 (s, 1H). MS (ESI) (M+H)⁺ 557.

### Example 30

### 6-Hydroxycarbamoylmethoxy-3-[(4-[1,2,3]triazol-1-ylmethyl-naphthalene-1-carbonyl)-amino]-pyridine-2-carboxylic Acid Cyclobutylmethyl-amide

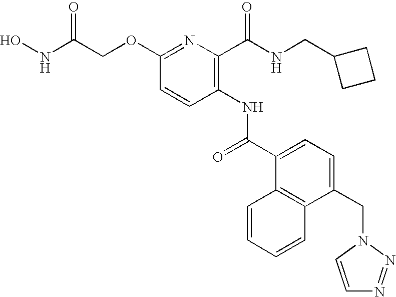

Following the procedure described in Example 4, using [(6-{[(cyclobutylmethyl)amino]carbonyl}-5-{[4-(1H-1,2,3-triazol-1-ylmethyl)-1-naphthoyl]amino}pyridin-2-yl)oxy]acetic acid, which was prepared in Example 3, (40 mg, 0.078 mmol), and hydroxylamine hydrochloride (16 mg, 0.23 mmol) provided the title compound after purification by column chromatography on silica gel using CH₂Cl₂/MeOH (100:5 and 100:15) as eluent (25 mg, 61%).

¹H NMR (500 MHz, CD₃OD) δ (ppm) 1.69-1.79 (m, 2H), 1.82-1.95 (m, 2H), 2.03-2.12 (m, 2H), 2.54-2.64 (m, 1H), 3.36 (d, 2H), 4.80 (s, 2H), 6.12 (s, 2H), 7.16 (d, 1H), 7.45 (d, 1H), 7.56 (s, 1H), 7.58-7.63 (m, 2H), 7.74 (d, 2H), 7.84 (d, 1H), 8.07-8.12 (m, 1H), 8.44-8.49 (m, 1H). MS (ESI) (M+H)⁺ 530.02.

### Example 31

### 6-(Methoxycarbamoyl-methoxy)-3-[(4-[1,2,3]triazol-1-ylmethyl-naphthalene-1-carbonyl)-amino]-pyridine-2-carboxylic Acid Cyclobutylmethyl-amide

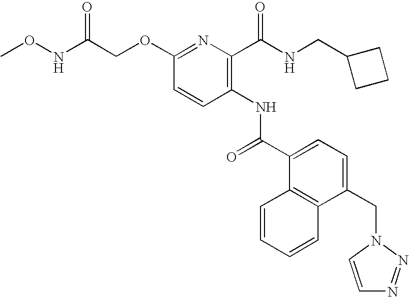

Following the procedure described in Example 4, using [(6-{[(cyclobutylmethyl)amino]carbonyl}-5-{[4-(1H-1,2,3-triazol-1-ylmethyl)-1-naphthoyl]amino}pyridin-2-yl)oxy]acetic acid, which was prepared in Example 3, (40 mg, 0.078 mmol), and methoxyamine hydrochloride (20 mg, 0.23 mmol) provided the title compound after workup (40 mg, 94%).

¹H NMR (500 MHz, CDCl₃) δ (ppm) 1.73-1.82 (m, 2H), 1.87-2.0 (m, 2H), 2.1-2.18 (m, 2H), 2.58-2.68 (m, 1H), 3.45 (“t”, 2H), 3.82 (s, 3H), 4.81 (s, 2H), 6.1 (s, 2H), 7.14 (d, 1H), 7.43 (s, 1H), 7.46 (d, 1H), 7.59-7.66 (m, 2H), 7.73 (s, 1H), 7.88 (d, 1H), 8.0-8.08 (m, 2H), 8.57 (d, 1H), 8.81 (br s, 1H), 9.46 (d, 1H), 12.72 (s, 1H). MS (ESI) (M+H)⁺ 544.

### Example 32

### {5-[(4-Methyl-naphthalene-1-carbonyl)-amino]-6-[(tetrahydro-pyran-4-ylmethyl)-carbamoyl]-pyridin-2-yloxy}-acetic Acid Methyl Ester

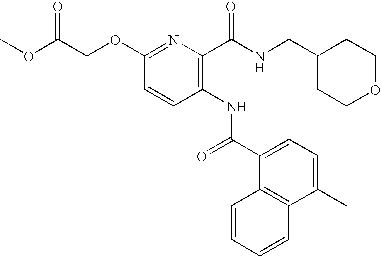

### Example 32A

### {5-[(4-Methyl-naphthalene-1-carbonyl)-amino]-6-[(tetrahydro-pyran-4-ylmethyl)-carbamoyl]-pyridin-2-yloxy}-acetic Acid Methyl Ester

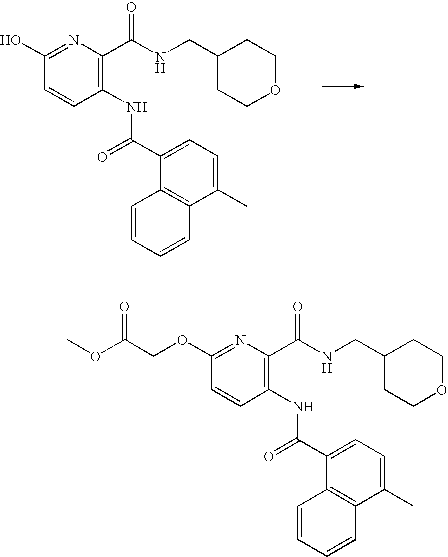

Following the procedure in described in Example 1A, using 6-Hydroxy-3-[(4-methyl-naphthalene-1-carbonyl)-amino]-pyridine-2-carboxylic acid (tetrahydro-pyran-4-ylmethyl)-amide (obtained from Example 32B) (150 mg, 0.36 mmol), and methyl bromoacetate (164 mg, 1.07 mmol) provided the title compound after purification by column chromatography on silica gel using CH₂Cl₂/MeOH (100:1) as eluent (110 mg, 63%). MS (ESI) (M+H)⁺ 492.01.

### Example 32B

### 6-Hydroxy-3-[(4-methyl-naphthalene-1-carbonyl)-amino]-pyridine-2-carboxylic Acid (tetrahydro-pyran-4-ylmethyl)-amide

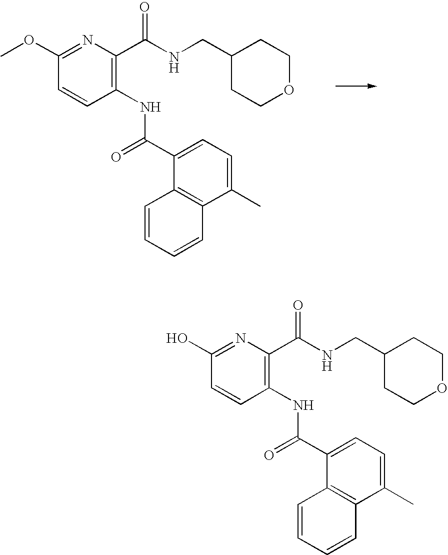

Following the procedure described in Example 2B, using 6-Methoxy-3-[(4-methyl-naphthalene-1-carbonyl)-amino]-pyridine-2-carboxylic acid (tetrahydro-pyran-4-ylmethyl)-amide (obtained from Example 32C) (685 mg, 1.58 mmol), and pyridine hydrochloride (14.5 g, 0.126 mol) provided the title compound after purification by column chromatography on silica gel using CH₂Cl₂/MeOH (100:2.5 and 100:5) as eluent (460 mg, 69%). MS (ESI) (M+H)⁺ 420.01.

### Example 32C

### 6-Methoxy-3-[(4-methyl-naphthalene-1-carbonyl)-amino]-pyridine-2-carboxylic Acid (tetrahydro-pyran-4-ylmethyl)-amide

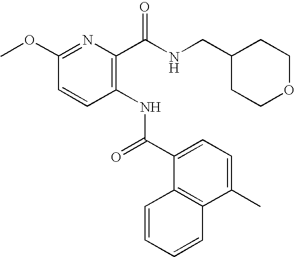

Following the procedure described in Example 2C, using methyl 6-methoxy-3-[(4-methyl-1-naphthoyl)amino]pyridine-2-carboxylate prepared in Example 1E (570 mg, 1.63 mmol), and 1-(tetrahydro-2H-pyran-4-yl)methanamine (1.75 g, 15.19 mmol) provided the title compound after workup (690 mg, 98%). MS (ESI) (M+H)⁺ 534.01.

### Example 33

### 6-Carbamoylmethoxy-3-[(4-methyl-naphthalene-1-carbonyl)-amino]-pyridine-2-carboxylic Acid (tetrahydro-pyran-4-ylmethyl)-amide

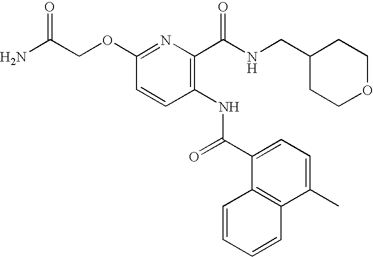

### Example 33A

### 6-Carbamoylmethoxy-3-[(4-methyl-naphthalene-1-carbonyl)-amino]-pyridine-2-carboxylic Acid (tetrahydro-pyran-4-ylmethyl)-amide

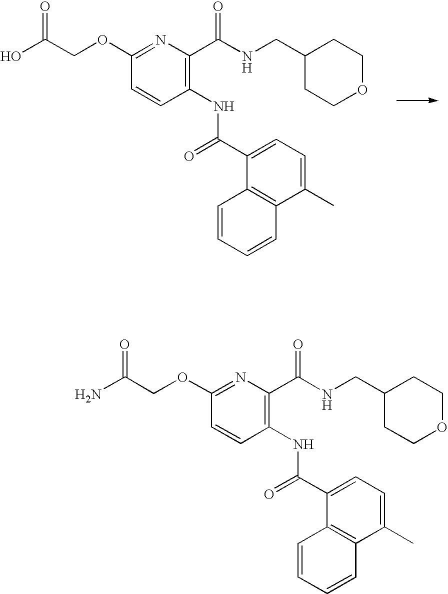

Following the procedure described in Example 4, using {5-[(4-Methyl-naphthalene-1-carbonyl)-amino]-6-[(tetrahydro-pyran-4-ylmethyl)-carbamoyl]-pyridin-2-yloxy}-acetic acid (obtained from Example 33B) (92 mg, 0.19 mmol), and ammonium chloride (52 mg, 0.96 mmol) provided the title compound after workup (87 mg, 95%).

¹H NMR (300 MHz, CDCl₃) δ (ppm) 1.3-1.46 (m, 2H), 1.6-1.72 (m, 2H), 1.77-1.93 (m, 1H), 2.74 (s, 3H), 3.29 (“t”, 2H), 3.38 (“t”, 2H), 3.91-4.04 (m, 2H), 4.74 (s, 2H), 5.59 (br s, 1H), 6.24 (br s, 1H), 7.12 (d, 1H), 7.39 (d, 1H), 7.53-7.62 (m, 2H), 7.79 (d, 1H), 8.0-8.16 (m, 2H), 8.52-8.60 (m, 1H), 9.44 (d, 1H), 12.53 (s, 1H). MS (ESI) (M+H)⁺ 477.02.

### Example 33B

### {5-[(4-Methyl-naphthalene-1-carbonyl)-amino]-6-[(tetrahydro-pyran-4-ylmethyl)-carbamoyl]-pyridin-2-yloxy}-acetic Acid

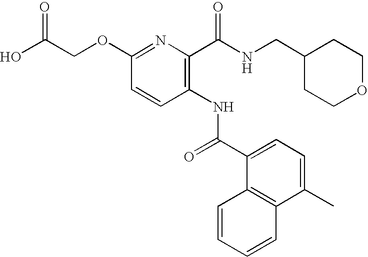

Following the procedure described in Example 3, using {5-[(4-Methyl-naphthalene-1-carbonyl)-amino]-6-[(tetrahydro-pyran-4-ylmethyl)-carbamoyl]-pyridin-2-yloxy}-acetic acid methyl ester (obtained from Example 32) (110 mg, 0.22 mmol), and NaOH (27 mg, 0.67 mmol) provided the title compound after workup (94 mg, 86%). MS (ESI) (M+H)⁺ 488.02.

### Example 34

### 6-(2-Hydroxy-ethoxy)-3-[(4-methyl-naphthalene-1-carbonyl)-amino]-pyridine-2-carboxylic Acid (tetrahydro-pyran-4-ylmethyl)-amide

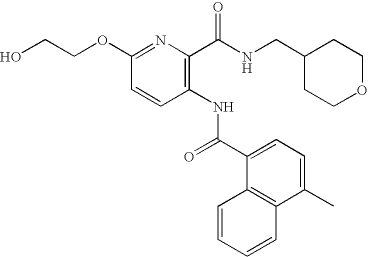

Following the procedure described in Example 1A, using 6-Hydroxy-3-[(4-methyl-naphthalene-1-carbonyl)-amino]-pyridine-2-carboxylic acid (tetrahydro-pyran-4-ylmethyl)-amide (obtained from Example 32B) (148 mg, 0.35 mmol), and 2-bromoethanol (220 mg, 1.75 mmol) provided the title compound after purification by column chromatography on silica gel using CH₂Cl₂/MeOH (100:2) as eluent (84 mg, 51%).

¹H NMR (500 MHz, CDCl₃) δ (ppm) 1.35-1.45 (m, 2H), 1.62-1.70 (m, 2H), 1.83-1.92 (m, 1H), 2.77 (s, 3H), 3.2 (“t”, 2H), 3.4 (“t”, 2H), 3.97-4.02 (m, 2H), 4.03-4.07 (m, 2H), 4.42-4.46 (m, 2H), 7.09 (d, 1H), 7.42 (d, 1H), 7.57-7.62 (m, 2H), 7.82 (d, 1H), 8.02-8.10 (m, 1H), 8.20 (t, 1H), 8.57-8.63 (m, 1H), 9.40 (d, 1H), 12.49 (s, 1H). MS (ESI) (M+H)⁺ 464.

### Example 35

### 6-(2-Hydroxy-ethoxy)-3-[(4-methoxymethyl-naphthalene-1-carbonyl)-amino]-pyridine-2-carboxylic Acid (tetrahydro-pyran-4-ylmethyl)-amide

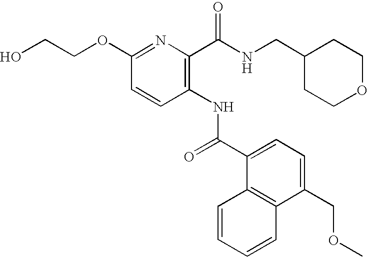

To a mixture of 6-(2-Hydroxy-ethoxy)-3-[(4-methyl-naphthalene-1-carbonyl)-amino]-pyridine-2-carboxylic acid (tetrahydro-pyran-4-ylmethyl)-amide (obtained from Example 34) (84 mg, 0.18 mmol) in CCl₄ (10 ml) was added N-bromosuccinimide (35 mg, 0.2 mmol) and benzoyl peroxide (9 mg, 0.04 mmol). The reaction mixture was refluxed for 3 h and then filtered. The filtrate was diluted with dichloromethane, washed with NaHCO₃ (aq, sat), dried and evaporated under reduced pressure. The residue was suspended in methanol (5 ml) and sodium thiomethoxide (50 mg) was added. The reaction mixture was stirred at room temperature for 72 h, and then the solvent was evaporated in vacuo. The residue was dissolved in dichloromethane, washed with 4 M HCl (aq), dried and evaporated under reduced pressure. The residue was purified by preparative HPLC using acetonitrile and ammonium acetate buffer (20:80 to 80:20) as eluent to give the title compound (4 mg, 5%).

¹H NMR (500 MHz, CDCl₃) δ (ppm) 1.25-1.45 (m, 2H), 1.55-1.7 (m, 2H), 1.80-1.90 (m, 1H), 3.27-3.32 (m, 2H), 3.33-4.0 (m, 2H), 3.48 (s, 3H), 3.94-4.05 (m, 4H), 3.39-4.43 (m, 2H), 4.95 (s, 2H), 7.07 (d, 1H), 7.55-7.61 (m, 3H), 7.84 (d, 1H), 8.10-8.14 (m, 1H), 8.17 (t, 1H), 8.51-8.57 (m, 1H), 9.38 (d, 1H), 12.50 (s, 1H). MS (ESI) (M+H)⁺ 494.02.

### Example 36 & 37

### 6-Methanesulfonyl-3-[(4-methyl-naphthalene-1-carbonyl)-amino]-pyridine-2-carboxylic Acid (tetrahydro-pyran-4-ylmethyl)-amide and 6-Methanesulfinyl-3-[(4-methyl-naphthalene-1-carbonyl)-amino]-pyridine-2-carboxylic Acid (tetrahydro-pyran-4-ylmethyl)-amide

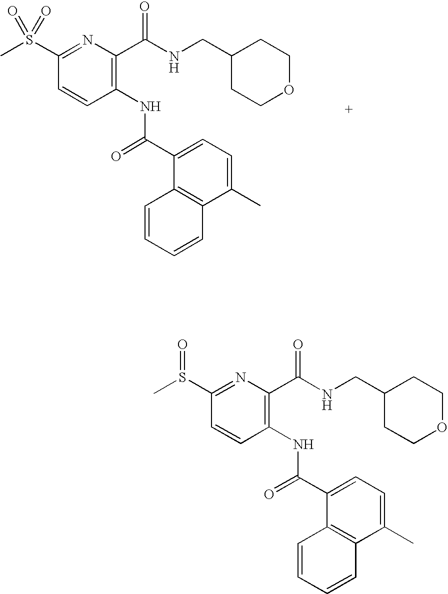

### Examples 36 & 37:A

### 6-Methanesulfonyl-3-[(4-methyl-naphthalene-1-carbonyl)-amino]-pyridine-2-carboxylic Acid (tetrahydro-pyran-4-ylmethyl)-amide and 6-Methanesulfinyl-3-[(4-methyl-naphthalene-1-carbonyl)-amino]-pyridine-2-carboxylic Acid (tetrahydro-pyran-4-ylmethyl)-amide

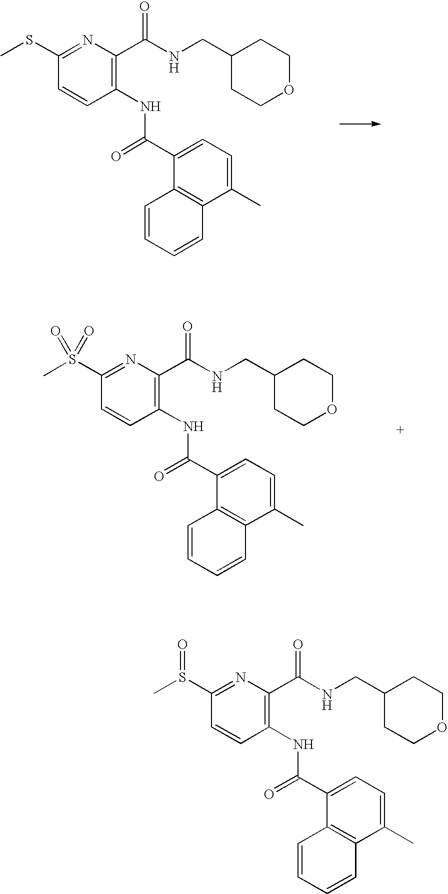

To a mixture of 3-[(4-Methyl-naphthalene-1-carbonyl)-amino]-6-methylsulfanyl-pyridine-2-carboxylic acid (tetrahydro-pyran-4-ylmethyl)-amide (obtained from Example 36 & 37:B) (0.5 g, 1.11 mmol) and K₂CO₃ (0.46 g, 3.34 mmol) in dichloromethane (10 ml) was added a solution of 3-chloroperbenzoic acid (0.39 g dissolved in 6 ml CH₂Cl₂) dropwise. The reaction mixture was stirred at room temperature for 70 min. More 3-chloroperbenzoic acid (70 mg dissolved in 3 ml CH₂Cl₂) was added and the reaction mixture was stirred for additional 20 min. The reaction mixture was diluted with dichloromethane. After addition of water, the organic phase was separated, washed with NaHCO₃ (aq, sat), dried and evaporated under reduced pressure. The residue was purified by column chromatography on silica gel using CH₂Cl₂/MeOH (100:2.5) as eluent and the title compounds were obtained:
6-Methanesulfonyl-3-[(4-methyl-naphthalene-1-carbonyl)-amino]-pyridine-2-carboxylic acid (tetrahydro-pyran-4-ylmethyl)-amide (191 mg, 36%):

¹H NMR (300 MHz, CDCl₃) δ (ppm) 1.30-1.46 (m, 2H), 1.60-1.70 (m, 2H), 1.80-1.96 (m, 1H), 2.77 (s, 3H), 3.19 (s, 3H), 3.30-3.43 (m, 4H), 3.93-4.02 (m, 2H), 7.44 (d, 1H), 7.57-7.65 (m, 2H), 7.83 (d, 1H), 8.04-8.12 (m, 1H), 8.24-8.34 (m, 2H), 8.55-8.63 (m, 1H), 9.69 (d, 1H), 13.02 (s, 1H).
And 6-Methanesulfinyl-3-[(4-methyl-naphthalene-1-carbonyl)-amino]-pyridine-2-carboxylic acid (tetrahydro-pyran-4-ylmethyl)-amide (312 mg, 60%):

¹H NMR (300 MHz, CDCl₃) δ (ppm) 1.24-1.48 (m, 2H), 1.58-1.72 (m, 2H), 1.78-1.96 (m, 1H), 2.77 (s, 3H), 2.85 (s, 3H), 3.29-3.43 (m, 4H), 3.93-4.03 (m, 2H), 7.43 (d, 1H), 7.56-7.65 (m, 2H), 7.83 (d, 1H), 8.04-8.12 (m, 1H), 8.16-8.27 (m, 2H), 8.55-8.64 (m, 1H), 9.69 (d, 1H), 12.84 (s, 1H). MS (ESI) (M+H)⁺ 466.

### Examples 36 & 37:B

### 3-[(4-Methyl-naphthalene-1-carbonyl)-amino]-6-methylsulfanyl-pyridine-2-carboxylic Acid (tetrahydro-pyran-4-ylmethyl)-amide

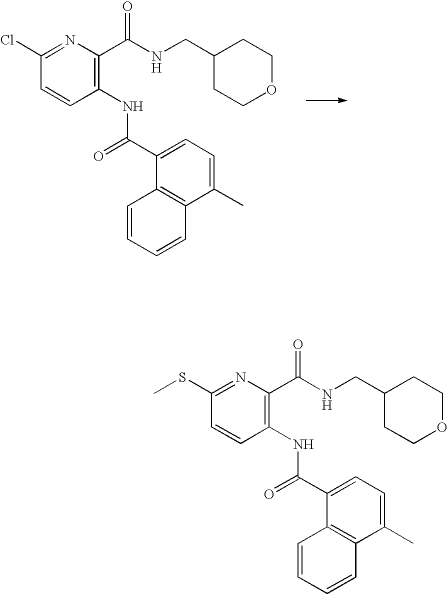

A mixture of 6-Chloro-3-[(4-methyl-naphthalene-1-carbonyl)-amino]-pyridine-2-carboxylic acid (tetrahydro-pyran-4-ylmethyl)-amide, obtained from Example 36 & 37:C (1 g, 2.28 mmol) and sodium thiomethoxide (0.48 g, 6.85 mmol) in anhydrous DMF (15 ml) was heated in the microwave at 100° C. for 15 min. Water was added at room temperature. The formed precipitate was collected, washed with water and air-dried. The solid was suspended in ether, and then removed by filtration and dried in vacuo to give the title compound (0.9 g, 94%). MS (ESI) (M+H)⁺ 449.97.

### Examples 36 & 37:C

### 6-Chloro-3-[(4-methyl-naphthalene-1-carbonyl)-amino]-pyridine-2-carboxylic Acid (tetrahydro-pyran-4-ylmethyl)-amide

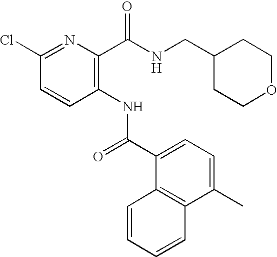

Following the procedure described in Example 2C, using methyl 6-chloro-3-[(4-methyl-1-naphthoyl)amino]pyridine-2-carboxylate prepared in Example 21D (3 g, 8.46 mmol), and 1-(tetrahydro-2H-pyran-4-yl)methanamine (4.87 g, 42.28 mmol) provided the title compound after workup (3.14 g, 85%). MS (ESI) (M+H)⁺ 438.

### Example 38

### 6-[2-(2-Hydroxy-ethoxy)-ethoxy]-3-[(4-[1,2,3]triazol-1-ylmethyl-naphthalene-1-carbonyl)-amino]-pyridine-2-carboxylic Acid (tetrahydro-pyran-4-ylmethyl)-amide

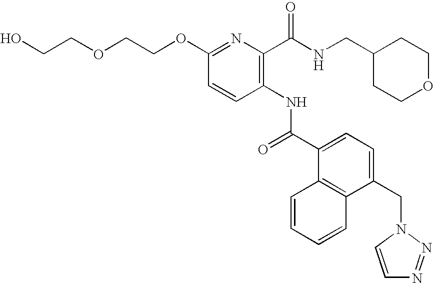

A mixture of 6-hydroxy-N-(tetrahydro-2H-pyran-4-ylmethyl)-3-{[4-(1H-1,2,3-triazol-1-ylmethyl)-1-naphthoyl]amino}pyridine-2-carboxamide prepared in Example 10B (62 mg, 0.13 mmol), 2-(2-chloro-ethoxy)-ethanol (116 mg, 0.93 mmol) and silver carbonate (171 mg, 0.62 mmol) in DMF (3 ml) was heated in microwave at 120° C. for 2.5 h. More 2-(2-chloro-ethoxy)-ethanol (800 mg) was added and the reaction mixture was heated in microwave at 130° C. for additional 4 h. The reaction mixture was diluted with dichloromethane and then filtered. The solvents were evaporated under reduced pressure. The residue was dissolved in dichloromethane, washed with water, dried, evaporated in vacuo, and then purified by preparative HPLC using acetonitrile and ammonium acetate buffer (20:80 to 70:30) as eluent to give the title compound (28 mg, 38%).

¹H NMR (300 MHz, CDCl₃) δ (ppm) 1.22-1.48 (m, 2H), 1.58-1.72 (m, 2H), 1.76-1.93 (m, 1H), 3.25-3.44 (m, 4H), 3.66-3.83 (m, 4H), 3.88-4.02 (m, 4H), 4.42-4.50 (m, 2H), 6.07 (s, 2H), 7.08 (d, 1H), 7.41 (s, 1H), 7.42 (d, 1H), 7.54-7.66 (m, 2H), 7.73 (s, 1H), 7.84 (d, 1H), 8.0 (d, 1H), 8.20 (t, 1H), 8.54 (d, 1H), 9.35 (d, 1H), 12.57 (s, 1H). MS (ESI) (M+H)⁺ 575.12.

### Example 39

### 6-methoxy-3-({-4-[(4-methylpiperazin-1-yl)methyl]-1-naphthoyl}amino)-N-(tetrahydro-2H-pyran-4-ylmethyl)pyridine-2-carboxamide

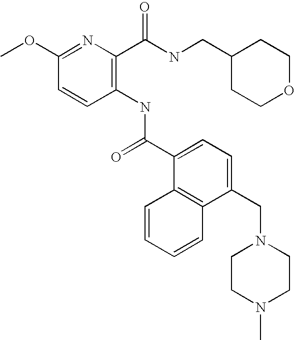

### Example 39A

### 6-methoxy-3-({4-[(4-methylpiperazin-1-yl)methyl]-1-naphthoyl}amino)-N-(tetrahydro-2H-pyran-4-ylmethyl)pyridine-2-carboxamide

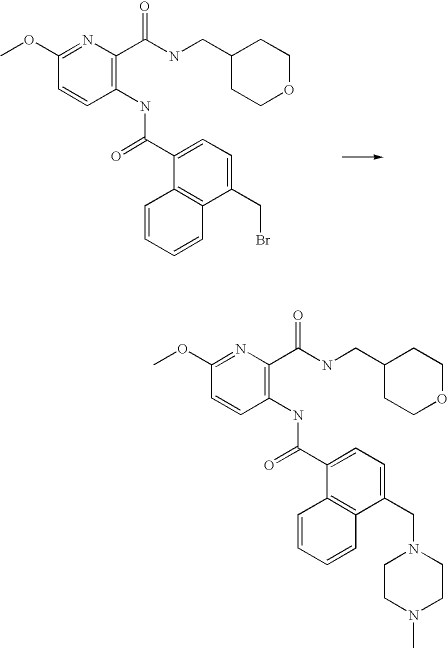

3-{[4-(Bromomethyl)-1-naphthoyl]amino}-6-methoxy-N-(tetrahydro-2H-pyran-4-ylmethyl)pyridine-2-carboxamide prepared in Example 39B (38.5 mg, 0.075 mmol) was added to a solution of NaH (7 mg, 0.29 mmol) and 1-methylpiperazine (15.6 mg, 0.16 mol) in acetonitrile (2 ml) and the reaction was stirred under nitrogen for 2 h. The mixture was diluted with water and DCM, dried over MgSO₄, filtered and concentrated under reduced pressure. The crude product was purified by flash silica gel chromatography using a 12+M Biotage column with Toluene:EtOH 30:1 with 10% Et₃N as eluent and thereafter preparative HPLC to give 1.2 mg (3%) of 6-methoxy-3-({4-[(4-methylpiperazin-1-yl)methyl]-1-naphthoyl}amino)-N-(tetrahydro-2H-pyran-4-ylmethyl)pyridine-2-carboxamide.

¹H NMR (400 MHz, CDCl₃) δ (ppm) 1.20-1.40 (m, 4H), 1.60-1.66 (m, 1H), 1.77-1.88 (m, 2H), 2.30-2.60 (m, 10H), 3.27-3.39 (m, 4H), 3.90-3.98 (m, 6H), 7.10 (d, 1H), 7.47-7.55 (m, 3H), 7.76 (s, 1H), 8.21-8.27 (m, 1H), 8.30-8.34 (m, 1H), 8.47-8.52 (m, 1H), 8.33 (d, 1H). MS (ESI) (M+H)⁺ 532.09.

### Example 39B

### 3-{[4-(bromomethyl)-1-naphthoyl]amino}-6-methoxy-N-(tetrahydro-2H-pyran-4-ylmethyl)pyridine-2-carboxamide

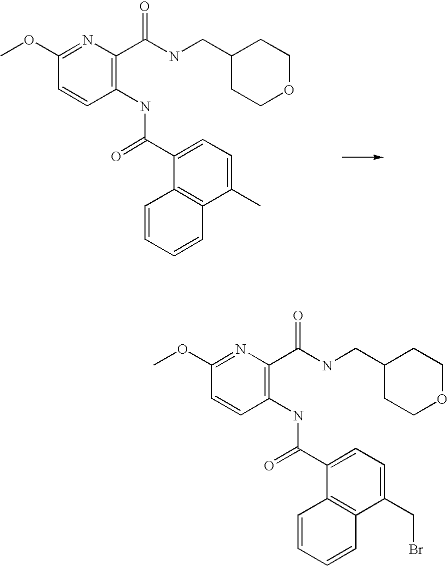

To a mixture of 3-{[4-(methyl)-1-naphthoyl]amino}-6-methoxy-N-(tetrahydro-2H-pyran-4-ylmethyl)pyridine-2-carboxamide prepared in Example 39C (144 mg, 0.33 mmol) in CCl₄ (7 ml) was added N-bromosuccinimide (65 mg, 0.36 mmol) and benzoyl peroxide (8 mg, 0.033 mmol). The reaction mixture was refluxed for 1.5 h under nitrogen. The organic solvent was removed under reduced pressure and the crude product was dissolved in DCM and filtered through a pad of silica by using Heptane:EtOAc 2:1 as eluent to give 143 mg (84%) of 3-{[4-(bromomethyl)-1-naphthoyl]amino}-6-methoxy-N-(tetrahydro-2H-pyran-4-ylmethyl)pyridine-2-carboxamide. MS (ESI) (M−H)⁻ 511.76.

### Example 39C

### 3-{[4-(methyl)-1-naphthoyl]amino}-6-methoxy-N-(tetrahydro-2H-pyran-4-ylmethyl)pyridine-2-carboxamide

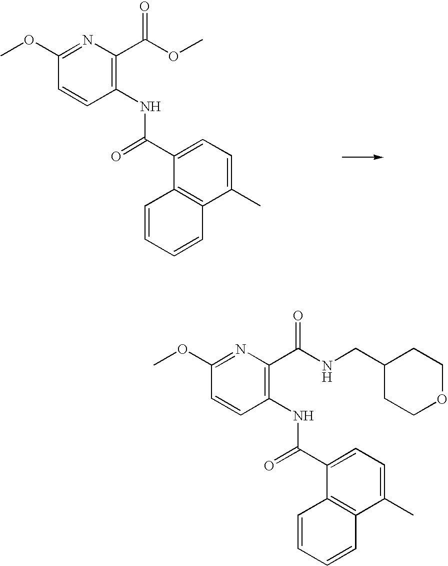

To a mixture of methyl 6-methoxy-3-[(4-methyl-1-naphthoyl)amino]pyridine-2-carboxylate prepared in Example 1E (1.0 g, 2.85 mmol) in dry DMF (20 ml) was 4-aminomethyltetrahydropyran (1.31 g, 11.42 mmol) added. The reaction mixture was stirred at 80° C. for 1.5 h under nitrogen. The reaction mixture was diluted with EtOAc and water. The organic phase was washed with 1M HCl, dried over anhydrous MgSO₄ and then filtered and concentrated under reduced pressure. The crude product was purified by flash silica gel chromatography using a 25+M Biotage column with Heptane:EtOAc 3:1 as eluent to give 1.2 g (97%) of 3-{[4-(methyl)-1-naphthoyl]amino}-6-methoxy-N-(tetrahydro-2H-pyran-4-ylmethyl)pyridine-2-carboxamide.

¹H NMR (400 MHz, CDCl₃) δ (ppm) 1.30-1.41 (m, 3H), 1.60-1.66 (m, 2H), 1.75-1.88 (m, 2H), 2.71 (s, 3H), 3.26-3.40 (m, 4H), 3.98 (s, 3H), 7.00 (d, 1H), 7.36 (d, 1H), 7.52-7.56 (m, 2H), 7.76 (d, 1H), 8.00-8.05 (m, 1H), 8.20-8.25 (m, 1H), 8.53-8.58 (m, 1H), 9.33 (s, 1H), 12.43 (s, 1H). MS (ESI) (+H)⁺ 434.05

### Example 40

### 6-methoxy-3-{[4-(morpholin-4-ylmethyl)-1-naphthoyl]amino}-N-(tetrahydro-2H-pyran-4-ylmethyl)pyridine-2-carboxamide

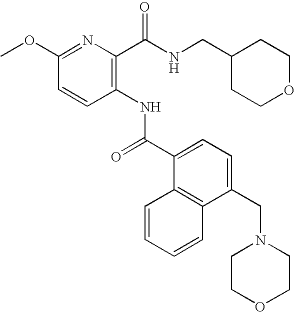

### Example 40A

### 6-methoxy-3-{[4-(morpholin-4-ylmethyl)-1-naphthoyl]amino}-N-(tetrahydro-2H-pyran-4-ylmethyl)pyridine-2-carboxamide

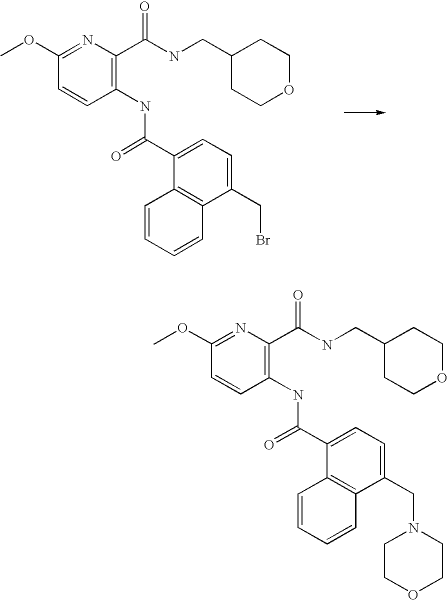

3-{[4-(Bromomethyl)-1-naphthoyl]amino}-6-methoxy-N-(tetrahydro-2H-pyran-4-ylmethyl)pyridine-2-carboxamide prepared in Example 39B (38.5 mg, 0.075 mmol) was added to a solution of NaH (7 mg, 0.29 mmol) and morpholine (13.6 mg, 0.16 mmol) in acetonitrile (2 ml) and the reaction was stirred under nitrogen for 2 h. The mixture was diluted with water and DCM, dried over MgSO₄, filtered and concentrated under reduced pressure. The crude product was purified by flash silica gel chromatography using a 12+M Biotage column with Toluene:EtOH 20:1 as eluent to give 34.5 mg (89%) of 6-methoxy-3-{[4-(morpholin-4-ylmethyl)-1-naphthoyl]amino}-N-(tetrahydro-2H-pyran-4-ylmethyl)pyridine-2-carboxamide.

¹H NMR (400 MHz, CDCl₃) δ (ppm) 0.97-1.04 (m, 1H), 1.32-1.44 (m, 3H), 1.71-1.78 (m, 2H), 1.95-2.07 (m, 1H), 2.66 (br s, 3H), 3.36-3.45 (m, 3H), 3.60 (s, 3H), 3.74-3.79 (m, 3H), 3.95-4.01 (m, 2H), 4.14 (s, 1H), 4.16 (s, 2H), 7.36 (d, 1H), 7.76-7.82 (m, 2H), 8.06 (d, 1H), 8.60-8.70 (m, 2H), 9.22-9.29 (m, 1H), 9.44 (d, 1H), 12.95 (s, 1H). MS (ESI) (M+H)⁺ 519.06.

### Example 41

### 6-[(ethylamino)sulfonyl]-3-{[4-(methoxymethyl)-1-naphthoyl]amino}-3-N-(tetrahydro-2H-pyran-4-ylmethyl)pyridine-2-carboxamide

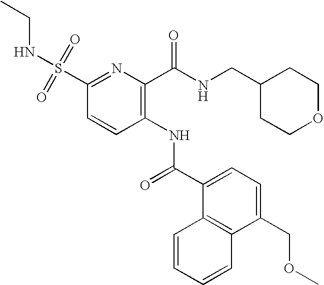

### Example 41A

### 6-[(ethylamino)sulfonyl]-3-{[4-(methoxymethyl)-1-naphthoyl]amino}-N-(tetrahydro-2H-pyran-4-ylmethyl)pyridine-2-carboxamide

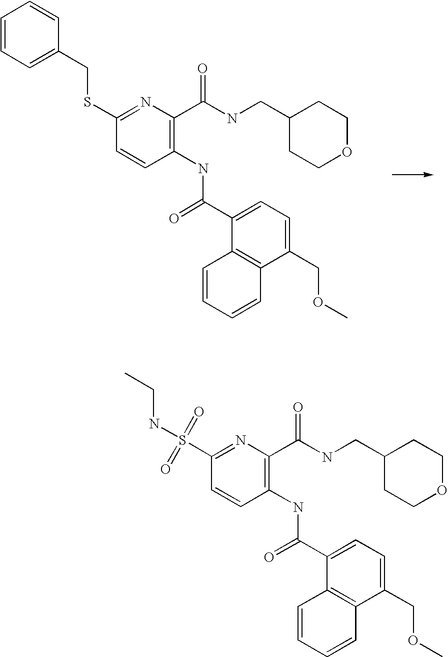

A solution of 6-(benzylthio)-3-{[4-(methoxymethyl)-1-naphthoyl]amino}-N-(tetrahydro-2H-pyran-4-ylmethyl)pyridine-2-carboxamide prepared from Example 41B (43 mg, 0.077 mmol) in DCM (1 ml), water (0.16 ml) and concentrated HCl (20 μl) was cooled to 0° C. Sodium hypochlorite (5%, 20 μl) was added dropwise to the solution and the reaction was stirred at 0° C. for 40 minutes to give the corresponding sulfonyl chloride. The resulting mixture was filtered through a phase separator into a reaction flask containing excess of ethylamine in DCM (1 ml). The organic solvent was reduced under reduced pressure and the crude product was purified by preparative HPLC to give 3.7 mg (9%) of 6-[(ethylamino)sulfonyl]-3-{[4-(methoxymethyl)-1-naphthoyl]amino}-N-(tetrahydro-2H-pyran-4-ylmethyl)pyridine-2-carboxamide.

¹H NMR (400 MHz, CDCl₃) δ (ppm) 1.13 (t, 3H), 1.29-1.40 (m, 2H), 1.59-1.65 (m, 2H), 1.80-1.90 (m, 1), 3.08-3.17 (m, 2H), 3.28-3.38 (m, 3H), 3.48 (s, 3H), 3.91-3.98 (m, 2H), 4.78-4.82 (m, 1H), 4.94 (s, 2H), 7.56-7.62 (m, 3H), 7.86 (d, 1H), 8.36-8.8.41 (m, 2H), 8.52-8.55 (m, 1H), 9.61 (d, 1H). MS (ESI) (M+H)⁺541.14.

### Example 41B

### 6-(benzylthio)-3-([4-(methoxymethyl)-1-naphthoyl]amino)-N-(tetrahydro-2H-pyran-4-ylmethyl)pyridine-2-carboxamide

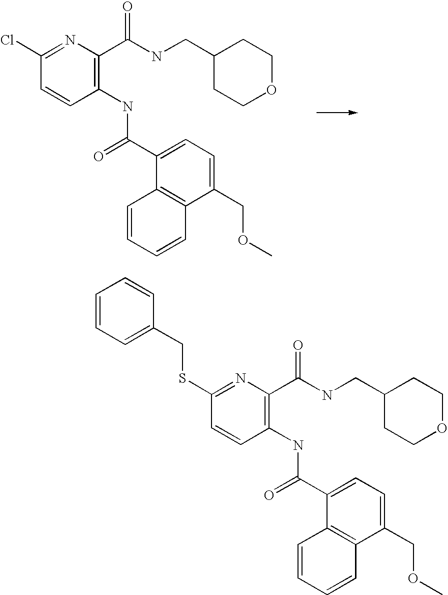

A solution of NaH (71.5 mg, 2.98 mmol) in DMF (25 ml) and benzyl mercaptan (370 mg, is 2.98 mmol) was added dropwise. After the addition was complete, 6-chloro-3-{[4-(methoxymethyl)-1-naphthoyl]amino}-N-(tetrahydro-2H-pyran-4-ylmethyl)pyridine-2-carboxamide prepared from Example 41C (465 mg, 0.99 mmol) was added. The reaction mixture was stirred at room temperature for 2 h. Water and DCM was added, the solution was filtered through a phase separator, concentrated under reduced pressure, purified by preparative HPLC to give 335 mg (61%) of 6-(benzylthio)-3-{[4-(methoxymethyl)-1-naphthoyl]amino}-N-(tetrahydro-2H-pyran-4-ylmethyl)pyridine-2-carboxamide.

¹H NMR (400 MHz, CDCl₃) δ (ppm) 1.19-1.30 (m, 3H), 1.60-1.75 (m, 1H), 3.15 (t, 2H), 3.25-3.39 (m, 3H), 3.45 (s, 3H), 3.87-3.95 (m, 2H), 4.35 (s, 2H), 4.92 (s, 2H), 7.12-7.17 (m, 1H), 7.29-7.34 (m, 2H), 7.38-7.43 (m, 3H), 7.54-57 (m, 3H), 7.80 (s, 1H), 7.96-8.02 (m, 1H), 8.08-8.13 (m, 1H), 8.47-8.54 (m, 1H), 9.24 (d, 1H), 12.54 (s, 1H). MS (ESI) (M−H)⁻ 554.04.

### Example 41C

### 6-chloro-3-{[4-(methoxymethyl)-1-naphthoyl]amino}-N-(tetrahydro-2H-pyran-4-ylmethyl)pyridine-2-carboxamide

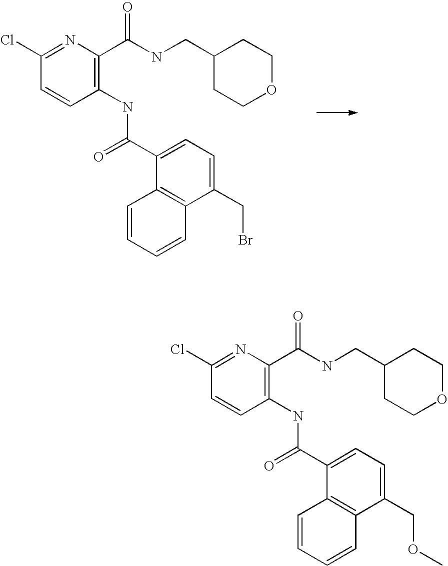

A solution of 3-{[4-(bromomethyl)-1-naphthoyl]amino}-6-chloro-N-(tetrahydro-2H-pyran-4-ylmethyl)pyridine-2-carboxamide prepared from Example 41D (1.27 g, 2.47 mmol) in sodium methoxide was stirred at room temperature over night. The organic solvent was removed under reduced pressure and the residue was diluted in DCM and water, the organic phase was dried, concentrated and purified by flash silica gel chromatography using a 25+M Biotage column with Toluene:EtOAc 9:1 as eluent to give 0.47 g (40%) of 6-chloro-3-{[4-(methoxymethyl)-1-naphthoyl]amino}-N-(tetrahydro-2H-pyran-4-ylmethyl)pyridine-2-carboxamide.

¹H NMR (400 MHz, CDCl₃) δ (ppm) 1.29-1.41 (m, 2H), 1.60-1.67 (m, 2H), 1.80-1.90 (m, 1H), 3.25-3.39 (m, 4H), 3.46 (s, 3H), 3.92-3.99 (m, 2H), 4.94 (s, 3H), 7.50 (d, 1H), 7.55-7.60 (m, 3H), 7.83 (d, 1H), 8.09-8.14 (m, 1H), 8.21-8.27 (m, 1H), 8.46-8.54 (m, 1H), 8.49-8.55 (m, 1H), 9.42 (d, 1H), 12.68 (s, 1H). MS (ESI) (M+H)⁺ 468.21.

### Example 41D

### 3-{[4-(bromomethyl)-1-naphthoyl]amino}-6-chloro-N-(tetrahydro-2H-pyran-4-ylmethyl)pyridine-2-carboxamide

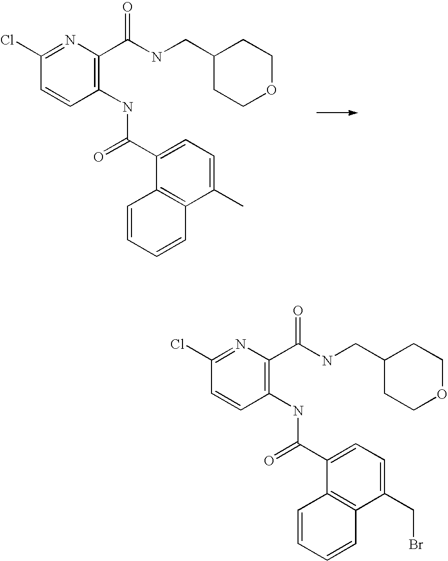

3-{[4-(bromomethyl)-1-naphthoyl]amino}-6-chloro-N-(tetrahydro-2H-pyran-4-ylmethyl)pyridine-2-carboxamide was prepared in the same way as in Example 39B in 100% yield. MS (ESI) (M+H)⁺ 518.00.

### Example 41E

### 6-chloro-3-[(4-methyl-1-naphthoyl)amino]-N-(tetrahydro-2H-pyran-4-ylmethyl)pyridine-2-carboxamide

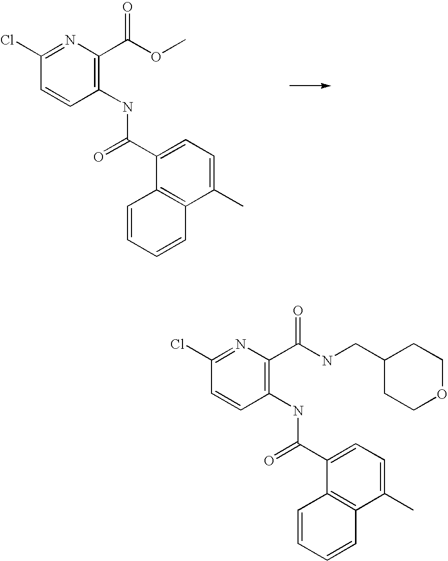

Methyl 6-chloro-3-[(4-methyl-1-naphthoyl)amino]pyridine-2-carboxylate was prepared in the same way as Example 21D and 6-chloro-3-[(4-methyl-1-naphthoyl)amino]-N-(tetrahydro-2H-pyran-4-ylmethyl)pyridine-2-carboxamide was prepared in the same way as in Example 39C. The crude compound was purified by flash silica gel chromatography using a Biotage column with Heptane:EtOAc 2:1 as eluent to give 93% yield.

¹H NMR (400 MHz, CDCl₃) δ (ppm) 1.29-1.41 (m, 2H), 1.60-1.66 (m, 2H), 1.80-1.90 (m, 1H), 2.73 (s, 3H), 3.26-3.40 (m, 4H), 3.92-3.99 (m, 2H), 7.38 (s, 1H), 7.50 (d, 1H), 7.54-7.59 (m, 1H), 7.78 (d, 2H), 8.02-8.07 (m, 1H), 8.20-8.27 (m, 1H), 8.51-8.57 (m, 1H), 9.41 (d, 1H), 12.68 (s, 1H). MS (ESI) (M+H)⁺ 438.07.

### Example 42 & 43

### 6-(benzylsulfonyl)-3-{[4-(methoxymethyl)-1-naphthoyl]amino}-N-(tetrahydro-2H-pyran-4-ylmethyl)pyridine-2-carboxamide and 6-(benzylsulfonyl)-3-{[4-(methoxymethyl)-1-naphthoyl]amino}-N-(tetrahydro-2H-pyran-4-ylmethyl)pyridine-2-carboxamide

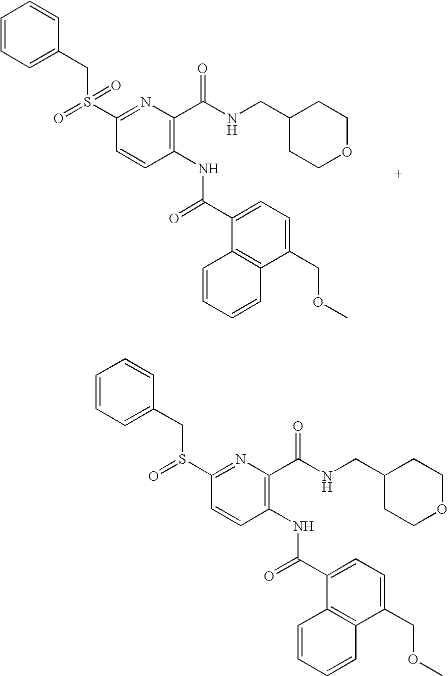

### Examples 42 & 43:A

### 6-(benzylsulfonyl)-3-{[4-(methoxymethyl)-1-naphthoyl]amino}-N-(tetrahydro-2H-pyran-4-ylmethyl)pyridine-2-carboxamide and 6-(benzylsulfonyl)-3-{[4-(methoxymethyl)-1-naphthoyl]amino}-N-(tetrahydro-2H-pyran-4-ylmethyl)pyridine-2-carboxamide

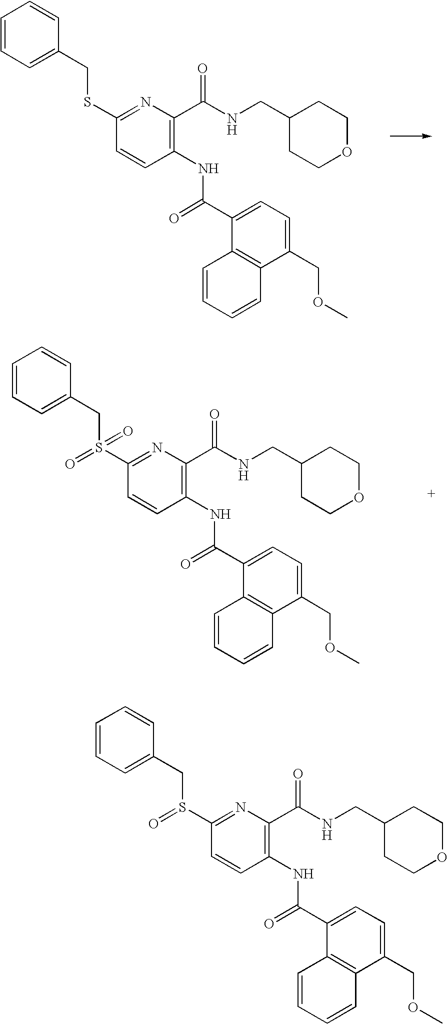

A solution of 6-(benzylthio)-3-{[4-(methoxymethyl)-1-naphthoyl]amino}-N-(tetrahydro-2H-pyran-4-ylmethyl)pyridine-2-carboxamide prepared from Example 41B (124 mg, 0.22 mmol) in DCM (3 ml) was 3-chloroperbenzoic acid (58 mg, 0.33 mmol) in chloroform (1 ml) added drop wise at 0° C. The reaction mixture was stirred at 0° C. for 30 min. Water was added and the solution was filtered through a phase separator, concentrated under reduced pressure and purified by preparative HPLC to give 65 mg (51%) of 6-(benzylsulfinyl)-3-{[4-(methoxymethyl)-1-naphthoyl]amino}-N-(tetrahydro-2H-pyran-4-ylmethyl)pyridine-2-carboxamide and 15 mg (12%) of 6-(benzylsulfonyl)-3-{[4-(methoxymethyl)-1-naphthoyl]amino}-N-(tetrahydro-2H-pyran-4-ylmethyl)pyridine-2-carboxamide. 6-(benzylsulfinyl)-3-{[4-(methoxymethyl)-1-naphthoyl]amino}-N-(tetrahydro-2H-pyran-4-ylmethyl)pyridine-2-carboxamide;

¹H NMR (400 MHz, CDCl₃) δ (ppm) 1.27-1.40 (m, 3H), 1.58-1.62 (m, 1H), 1.75-1.86 (m, 1H), 3.13-3.21 (m, 1H), 3.30-3.40 (m, 3H), 3.48 (s, 3), 3.92-4.00 (m, 2H), 4.14-4.24 (m, 2H), 4.93 (s, 3H), 6.97-7.02 (m, 2H), 7.25-7.30 (m, 3H), 7.55-7.61 (m, 3H), 7.84 (dd, 2H), 7.99-8.05 (m, 1H), 8.09-8.14 (m, 1H), 8.51-8.57 (m, 1H), 9.95 (d, 1H), 12.84 (s, 1H). MS (ESI) (M+H)⁺ 572.33.
And 6-(benzylsulfonyl)-3-{[4-(methoxymethyl)-1-naphthoyl]amino}-N-(tetrahydro-2H-pyran-4-ylmethyl)pyridine-2-carboxamide;

¹H NMR (400 MHz, CDCl₃) δ (ppm) 1.28-1.40 (m, 3H), 1.58-1.62 (m, 1H), 1.87-1.89 (m, 1H), 3.25-3.40 (m, 4H), 3.48 (s, 3H), 3.92-3.99 (m, 2H), 4.49 (s, 2H), 4.94 (s, 2H), 7.12-7.17 (m, 2H), 7.25-7.35 (m, 3H), 7.55-7.63 (m, 3H), 7.90 (dd, 2H), 8.09-8.18 (m, 2H), 8.50-8.56 (m, 1H), 9.51 (d, 2H), 13.02 (s, 1H). MS (ESI) (M+H)⁺ 588.34.

### Example 44

### 6-[(Tetrahydro-2H-pyran-4-ylmethyl)carbamoyl]-5-{[4-(1H-1,2,3-triazol-1-ylmethyl)-1-naphthoyl]amino}pyrazin-2-yl 3,3,3-trifluoropropane-1-sulfonate

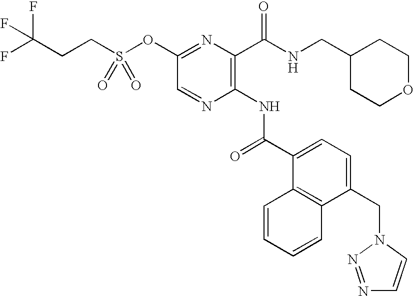

### Example 44A

### 6-[Tetrahydro-2H-pyran-4-ylmethyl)carbamoyl]-5-{[4-(1H-1,2,3-triazol-1-ylmethyl)-1-naphthoyl]amino}pyrazin-2-yl 3,3,3-trifluoropropane-1-sulfonate

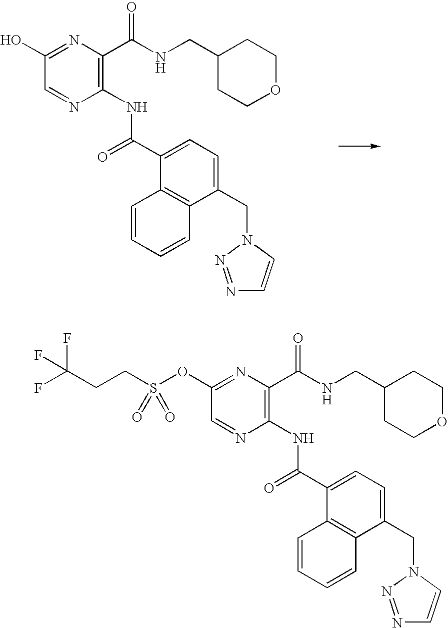

Silver carbonate (30 mg, 0.108 mmol) was added to a solution of 6-hydroxy-N-(tetrahydro-2H-pyran-4-ylmethyl)-3-{[4-(1H-1,2,3-triazol-1-ylmethyl)-1-naphthoyl]amino}pyrazine-2-carboxamide (15 mg, 0.031 mmol), prepared in Example 44B, in acetonitrile (10 ml) and the mixture was stirred for 5 min. Then, 3,3,3-trifluoropropane-1-sulfonyl chloride (18 mg, 0.092 mmol) was added and the reaction mixture was heated at reflux for 2.5 h. The reaction was quenched by the addition of a mixture of CH₂Cl₂/MeOH (1:1, 10 ml), the solid material was filtered off, and the filtrate was evaporated. The residue was dissolved in CH₂Cl₂, washed with saturated aqueous NaHCO₃ and saturated aqueous NaCl, dried (Na₂SO₄), and evaporated. Flash column chromatography (CH₂Cl₂/MeOH 40:1) of the residue gave 6-[(tetrahydro-2H-pyran-4-ylmethyl)carbamoyl]-5-{[4-(1H-1,2,3-triazol-1-ylmethyl)-1-naphthoyl]amino}pyrazin-2-yl 3,3,3-trifluoropropane-1-sulfonate (20 mg, quantitative):

¹H NMR (600 MHz, CDCl₃) δ (ppm) 1.33-1.40 (m, 2H), 1.60-1.64 (m, 2H), 1.80-1.88 (m, 1H), 2.89-2.97 (m, 2H), 3.31-3.39 (m, 4H), 3.68-3.72 (m, 2H), 3.95-3.99 (m, 2H), 6.09 (s, 2H), 7.41 (d, 1H), 7.43 (s, 1H), 7.60-7.65 (m, 2H), 7.71 (s, 1H), 7.79 (br t, 1H), 7.90 (d, 1H), 8.03-8.05 (m, 1H), 8.61-8.63 (m, 1H), 8.69 (s, 1H), 12.63 (s, 1H); MS (ESI) (M+H)⁺ 648.0.

### Example 44B

### 6-Hydroxy-N-(tetrahydro-2H-pyran-4-ylmethyl)-3-{[4-(1H-1,2,3-triazol-1-ylmethyl)-1-naphthoyl]amino}pyrazine-2-carboxamide

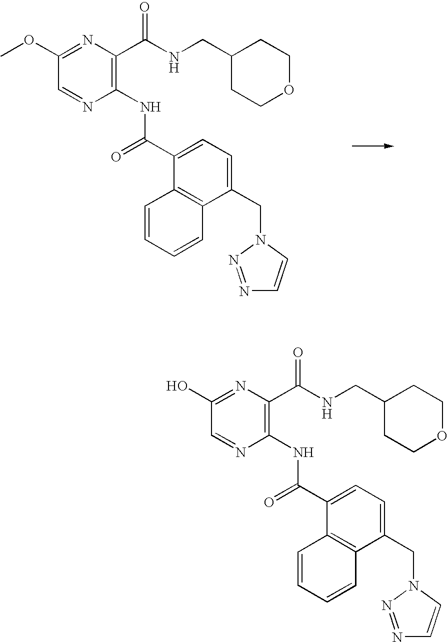

A mixture of 6-methoxy-N-(tetrahydro-2H-pyran-4-ylmethyl)-3-{[4-(1H-1,2,3-triazol-1-ylmethyl)-1-naphthoyl]amino}pyrazine-2-carboxamide prepared in Example 44C (142 mg, 0.283 mmol) and pyridine hydrochloride (2.78 g, 24.1 mmol) was heated at 150° C. for 2.5 h. Water was added at room temperature and the formed precipitate was collected, washed with water and dried. The filtrate was cooled to 4° C. over night and the additionally formed precipitate was collected, washed with water and dried. The combined precipitate was purified by revered-phase HPLC (20-100% MeCN in 0.1M aqueous NH₄OAc) to give 6-hydroxy-N-(tetrahydro-2H-pyran-4-ylmethyl)-3-{[4-(1H-1,2,3-triazol-1-ylmethyl)-1-naphthoyl]amino}pyrazine-2-carboxamide (15 mg, 11%):

MS (ESI) (+H)⁺ 488.0.

### Example 44C

### 6-Methoxy-N-(tetrahydro-2H-pyran-4-ylmethyl)-3-{[4-(1H-1,2,3-triazol-1-ylmethyl)-1-naphthoyl]amino}pyrazine-2-carboxamide

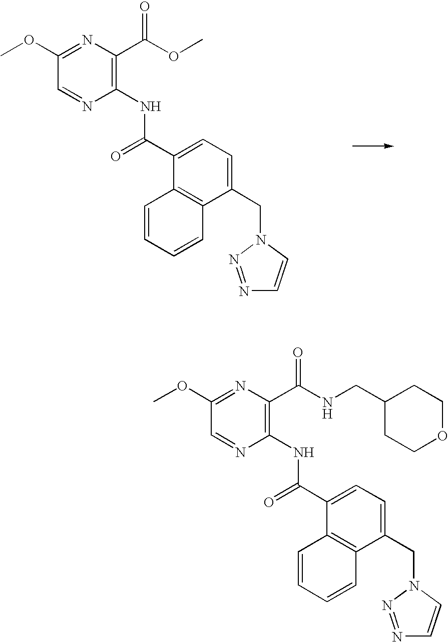

A solution of the corresponding TFA salt of methyl 6-methoxy-3-{[4-(1H-1,2,3-triazol-1-ylmethyl)-1-naphthoyl]amino}pyrazine-2-carboxylate prepared in Example 44D (185 mg, 0.295 mmol) and 1-(tetrahydro-2H-pyran-4-yl)methanamine (258 mg, 2.24 mmol) in DMF (3 ml) was heated at 120° C. over night (14 h). Additional 1-(tetrahydro-2H-pyran-4-yl)methanamine (150 mg, 1.30 mmol) was added and the reaction mixture was stirred at 90° C. for a further 16 h. The solution was then evaporated under reduced pressure, and the residue purified by revered-phase HPLC (30→100% MeCN in 0.1M aqueous NH₄OAc) to give 6-methoxy-N-(tetrahydro-2H-pyran-4-ylmethyl)-3-{[4-(1H-1,2,3-triazol-1-ylmethyl)-1-naphthoyl]amino}pyrazine-2-carboxamide (158 mg, quantitative):

¹H NMR (400 MHz, CDCl₃) δ (ppm) 1.30-1.41 (m, 2H), 1.59-1.65 (m, 2H), 1.78-1.89 (m, 1H), 3.29-3.39 (m, 4H), 3.93-4.00 (m, 2H), 4.00 (s, 3H), 6.05 (s, 2H), 7.37 (s, 1H), 7.40 (d, 1H), 7.54-7.61 (m, 2H), 7.67 (s, 1H), 7.87 (d, 1H), 7.94-8.00 (m, 2H), 8.44 (s, 1H), 8.59-8.63 (m, 1H), 12.19 (s, 1H); MS (ESI) (M+H)⁺ 502.0.

### Example 44D

### Methyl 6-methoxy-3-{[4-(1H-1,2,3-triazol-1-ylmethyl)-1-naphthoyl]amino}pyrazine-2-carboxylate

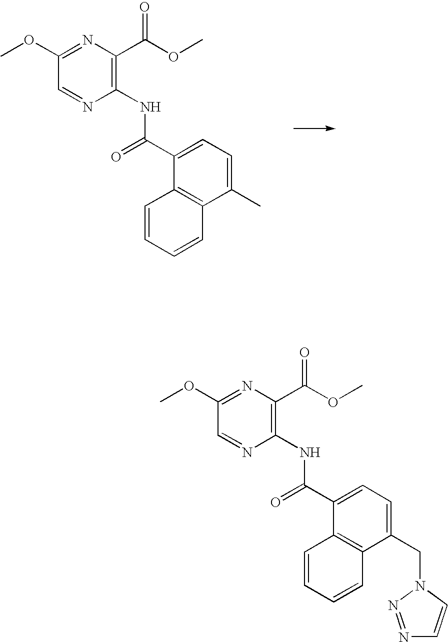

N-Bromosuccinimine (456 mg, 2.56 mmol) and benzoylperoxide (61 mg, 0.25 mmol) was added to a warm (˜77° C.) suspension of methyl 6-methoxy-3-[(4-methyl-1-naphthoyl)amino]pyrazine-2-carboxylate, prepared in Example 25F or alternatively in Example 44E, (883 mg, 2.51 mmol) in CCl₄ (60 ml). The resulting reaction mixture was heated at reflux for 2.5 h. Additional amount of benzoylperoxide (catalytic, tip of a spatula) was added and reaction mixture was heated at reflux for a further 12 h. After removal of solvents, the residue was dissolved in EtOAc, washed with water and saturated aqueous NaCl, dried (Na₂SO₄), and evaporated to give crude benzyl bromide (1.14 g). To this crude benzyl bromide (1.13 g) dissolved in acetonitrile (50 ml) at reflux was added 1,2,3-triazole (0.487 ml, 8.40 mmol) and the resulting mixture was heated at reflux over night (16 h). The solution was then evaporated under reduced pressure, and the residue was purified by revered-phase HPLC (30→100% MeCN in 0.1M aqueous NH₄OAc; followed by 30→100% MeCN in 0.15% aqueous TFA) to give methyl 6-methoxy-3-{[4-(1H-1,2,3-triazol-1-ylmethyl)-1-naphthoyl]amino}pyrazine-2-carboxylate as the corresponding TFA salt (188 mg—pure and 333 mg—slightly contaminated, ˜39% overall yield): MS (ESI) (M+H)⁺ 418.9.

### Example 44E

### Methyl-6-methoxy-3-[(4-methyl-1-naphthoyl)amino]pyrazine-2-carboxylate

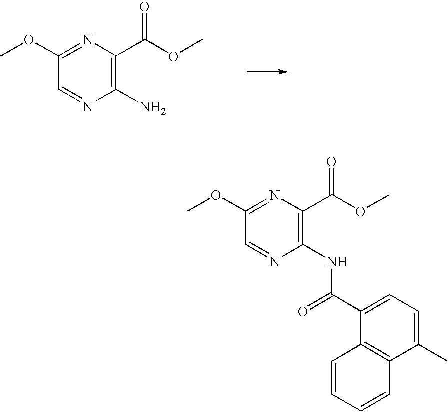

A solution of 4-methyl-1-naphthoyl chloride (3.37 g, 16.5 mmol) in CHCl₃ (6 ml) was added to a mixture of methyl-3-amino-6-methoxypyrazine-2-carboxylate (604 mg, 3.30 mmol) obtained from Example 25G and 4-dimethylaminopyridine (40 mg, 0.33 mg) in pyridine (10 ml). The resulting reaction mixture was stirred at 50° C. over night (14 h). NaHCO₃(s) (1.39 g, 16.5 mmol) was then added and after the evolution of gas had ceased the reaction mixture was evaporated. The residue was partitioned between CH₂Cl₂ and water and the organic phase was then washed with saturated aqueous NaHCO₃ and water, dried Na₂SO₄), and evaporated under reduced pressure. Flash column chromatography (toluene/EtOH 30:1) of the residue gave the diacylated pyrazine derivative (1.48 g): MS (ESI) (M+H)⁺ 519.9.

This diacylated pyrazine derivative (1.48 g) was dissolved in 1,4-dioxane (10 ml) and 2-propanol (6 ml) at ˜100° C. Hydrazine monohydrate (0.138 ml, 2.85 mmol) was added and the reaction mixture heated at reflux for 30 min. The reaction mixture was then evaporated under reduced pressure and the residue subjected to flash column chromatography (toluene/EtOH 15:1) to give methyl 6-methoxy-3-[(4-methyl-1-naphthoyl)amino]pyrazine-2-carboxylate (461 mg, overall yield 76%): MS (ESI) (M+H)⁺ 352.1.

### Example 45 & 46

### N-(Cyclobutylmethyl)-3-{[4-({5-[(dimethylamino)methyl]-1H-1,2,3-triazol-1-yl}methyl)-1-naphthoyl]amino}pyridine-2-carboxamide and N-(Cyclobutylmethyl)-3-{[4-({4-[(dimethylamino)methyl]-1H-1,2,3-triazol-1-yl}methyl)-1-naphthoyl]amino}pyridine-2-carboxamide

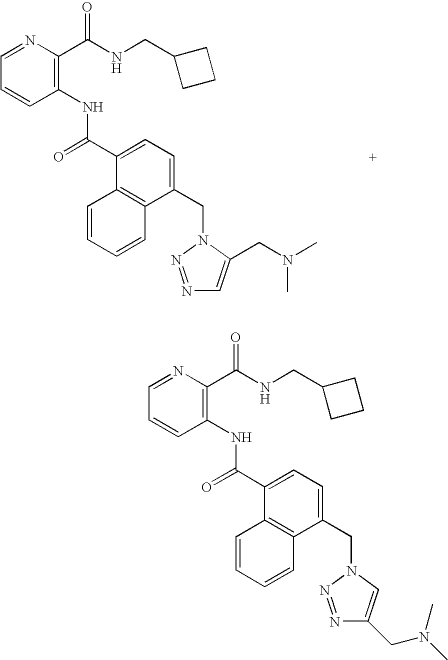

Using procedures analogous to Examples 15&16:A-B, using N,N-dimethylprop-2-yn-1-amine (0.298 ml, 2.77 mmol) and crude methyl 3-{[4-(azidomethyl)-1-naphthoyl]amino}pyridine-2-carboxylate (200 mg, 0.553 mmol), which was obtained from Example 15&16C, provided:
N-(cyclobutylmethyl)-3-{[4-({5-[(dimethylamino)methyl]-1H-1,2,3-triazol-1-yl}methyl)-1-naphthoyl]amino}pyridine-2-carboxamide (41 mg, 15% from crude azide):

¹H NMR (500 MHz, CD₃CN) δ ppm) 1.73-1.81 (m, 2H), 1.84-1.95 (m, 2H), 2.20 (s, 6H), 2.55-2.65 (m, 1H), 3.37-3.41 (m, 2H), 3.47 (s, 2H), 6.20 (s, 2H), 7.23 (d, 1H), 7.63-7.73 (m, 4H), 7.86 (d, 1H), 8.33-8.37 (m, 1H), 8.53 (d, 1H), 8.61 (br s, 1H), 9.33 (d, 1H), 13.00 (br s, 1H); MS (ESI) (M+H)⁺ 498.1;
And N-(cyclobutylmethyl)-3-{[4-({4-[(dimethylamino)methyl]-1H-1,2,3-triazol-1-yl}methyl)-1-naphthoyl]amino}pyridine-2-carboxamide (50 mg, 18% from crude azide):

¹H NMR (400 MHz, CD₃CN) δ (ppm) 1.68-1.79 (m, 2H), 1.81-1.90 (m, 2H), 1.98-2.06 (m, 2H, 2.18 (s, 6H), 3.33-3.37 (m, 2H), 3.56 (s, 2H), 6.08 (s, 2H), 7.43 (d, 1H), 7.59-7.69 (m, 4H), 7.86 (d, 1H), 8.17-8.21 (m, 1H), 8.32 (dd, 1H), 8.47-8.51 (m, 1H), 8.58 (br s, 1H), 9.29 (dd, 1H), 12.98 (br s, 1H); MS (ESI) (M+H)⁺ 498.1.

### Example 47

### N-(Cyclobutylmethyl)-3-[(4-{[4-(trifluoromethyl)-1H-1,2,3-triazol-1-yl]methyl}-1-naphthoyl)amino]pyridine-2-carboxamide

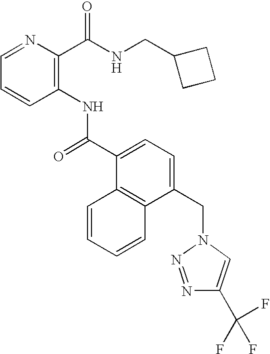

Using procedures analogous to Examples 15&16:A-B, using excess 3,3,3-trifluoroprop-1-yne (˜1 ml, condensed at −78° C.) and crude methyl 3-{[4-(azidomethyl)-1-naphthoyl]amino}pyridine-2-carboxylate (119 mg, 0.329 mmol), which was obtained from Example 15&16C, provided: N-(cyclobutylmethyl)-3-[(4-{[4-(trifluoromethyl)-1H-1,2,3-triazol-1-yl]methyl}-1-naphthoyl)amino]pyridine-2-carboxamide (20 mg, 12% from crude azide):

¹H NMR (400 MHz, CDCl₃) 1.68-1.77 (m, 2H), 1.83-1.94 (m, 2H), 2.03-2.12 (m, 2H), 2.50-2.62 (m, 1H), 3.37-3.42 (m, 2H), 6.06 (s, 2H), 7.49-7.54 (m, 2H), 7.57-7.64 (m, 3H), 7.88 (d, 1H), 7.93-7.98 (m, 1H), 8.28 (d, 1H), 8.40-8.48 (m, 1H), 8.53-8.57 (m, 1H), 9.36 (d, 1H), 12.95 (br s, 1H); MS (ESI) (+H)⁺ 509.0.

### Example 48 & 49

### N-(Cyclobutylmethyl)-3-[(4-{[5-(phenylsulfonyl)-1H-1,2,3-triazol-1-yl]methyl}-1-naphthoyl)amino]pyridine-2-carboxamide and N-(cyclobutylmethyl)-3-[(4-{[4-(phenylsulfonyl)-1H-1,2,3-triazol-1-yl]methyl}-1-naphthoyl)amino]pyridine-2-carboxamide

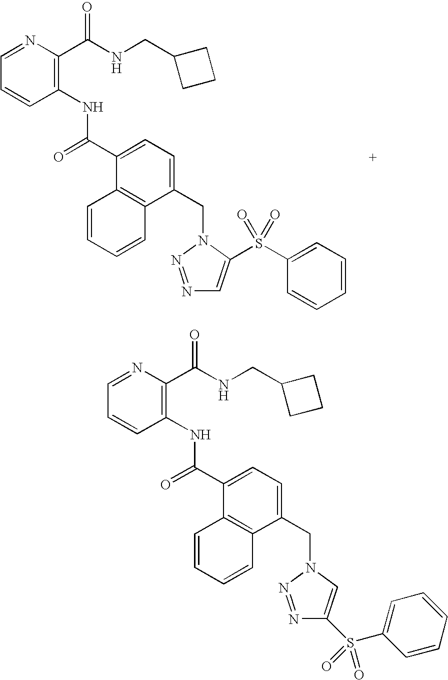

Using procedures analogous to Examples 15&16:A-B, using 1-fluorovinyl phenyl sulfone, obtained according to the procedure of Matthews et al. [Matthews; McCarthy; J. Org. Chem.; 1990, 55, 2973-2975] (235 mg, 1.26 mmol) and crude methyl 3-{[4-(azidomethyl)-1-naphthoyl]amino}pyridine-2-carboxylate (228 mg, 0.631 mmol), which was obtained from Example 15&16:C, provided:
N-(Cyclobutylmethyl)-3-[(4-{[5-(phenylsulfonyl)-1H-1,2,3-triazol-1-yl]methyl}-1-naphthoyl)amino]pyridine-2-carboxamide (75 mg, 20% from crude azide):

¹H NMR (500 MHz, CDCl₃) δ (ppm) 1.73-1.81 (m, 2H), 1.87-1.99 (m, 2H), 2.08-2.15 (m, 2H), 2.56-2.65 (m, 1H), 3.43-3.46 (m, 2H), 6.30 (d, 1H), 6.37 (br s, 2H), 7.14-7.17 (m, 2H), 7.38 (d, 1H), 7.41-7.45 (m, 1H), 7.45-7.48 (m, 2H), 7.55 (dd, 1H), 7.66-7.72 (m, 2H), 8.04-8.06 (m, 1H), 8.31 (dd, 1H), 8.36 (s, 1H), 8.43-8.47 (m, 1H), 8.55-8.58 (m, 1H), 9.39 (dd, 1H), 12.86 (br s, 1H); MS (ESI) (M+H)⁺ 581.0;
And N-(cyclobutylmethyl)-3-[(4-{[4-(phenylsulfonyl)-1H-1,2,3-triazol-1-yl]methyl}-1-naphthoyl)amino]pyridine-2-carboxamide (39 mg, 11% from crude azide):

¹H NMR (500 MHz, CDCl₃) δ (ppm) 1.69-1.77 (m, 2H), 1.84-1.96 (m, 2H), 2.05-2.12 (m, 2H), 2.52-2.61 (m, 1H), 3.39-3.43 (m, 2H), 6.02 (s, 2H), 7.48-7.54 (m, 3H), 7.55-7.63 (m, 3H), 7.87-7.92 (m, 2H), 8.01-8.04 (m, 2H), 8.29 (dd, 1H), 8.41-8.46 (m, 1H), 8.57 (d, 1H), 9.37 (dd, 1H), 12.98 (br s, 1H); MS (ESI) (M+H)⁺ 581.0.

### Example 50

### N-(Cyclobutylmethyl)-3-({4-[(4-fluoro-1H-1,2,3-triazol-1-yl)methyl]-1-naphthoyl}amino)pyridine-2-carboxamide

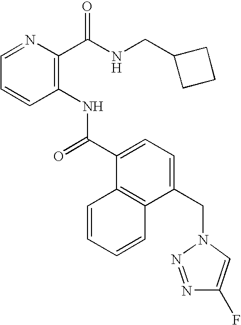

The compound was isolated as a by-product from the synthesis (see Example 47 & 48) of N-(cyclobutylmethyl)-3-[(4-{[5-(phenylsulfonyl)-1H-1,2,3-triazol-1-yl]methyl}-1-naphthoyl)amino]pyridine-2-carboxamide and N-(cyclobutylmethyl)-3-[(4-{[4-(phenylsulfonyl)-1H-1,2,3-triazol-1-yl]methyl}-1-naphthoyl)amino]pyridine-2-carboxamide (2 mg, ˜0.7% from crude azide):

¹H NMR (500 MHz, CDCl₃) δ (ppm) 1.64-1.72 (m, 2H), 1.79-1.91 (m, 2H), 2.00-2.08 (m, 2H), 2.47-2.56 (m, 1H), 3.33-3.37 (m, 2H), 5.91 (s, 2H), 6.99 (d, 1H), 7.44 (d, 1H), 7.47 (dd, 1H), 7.52-7.58 (m, 2H), 7.82 (d, 1H), 7.92-7.95 (m, 1H), 8.24 (dd, 1H), 8.37-8.42 (m, 1H), 8.48-8.51 (m, 1H), 9.32 (dd, 1H), 12.88 (br s, 1H); MS (ESI) (M+H)⁺ 459.0.

### Example 51

### N-{2-[(cyclobutylmethyl)carbamoyl]pyridin-3-yl}-1-methyl-1H-indole-3-carboxamide

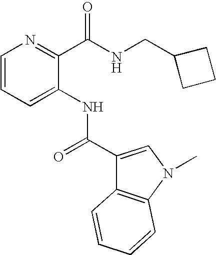

### Example 51A

### N-{2-[(cyclobutylmethyl)carbamoyl]pyridin-3-yl}-1-methyl-1H-indole-3-carboxamide

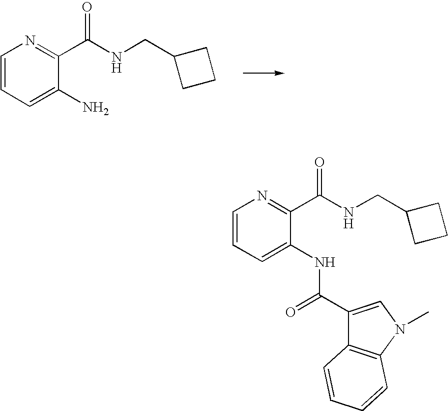

The title compound was prepared by applying general procedure 3 to 3-amino-N-(cyclobutylmethyl)pyridine-2-carboxamide, obtained from Example 51B. The acid chloride of 1H-indole-3-carboxylic acid was generated according general procedure 2. The reaction mixture was subjected to aqueous work-up (NaHCO₃) and the organic layer was separated and dried. The crude product was purified using silica based chromatography with an eluent system containing EtOAc/heptane (1:2) to afford the title compound (93%) as a colorless solid.

1H NMR (CDCl₃, 400 MHz) δ (ppm) 1.75-1.86 (m, 2H), 1.89-2.00 (m, 2H), 2.10-2.19 (m, 2H), 2.57-2.70 (m, 1H), 3.47-3.53 (m, 2H), 3.86 (s, 1H), 7.28-7.36 (m, 4H), 7.42 (d, 1H), 7.83 (s, 3H), 8.17 (dd, 1H), 8.41-8.50 (m, 2H), 9.31 (dd, 1H), 12.71 (bs, 1H);

### Example 51B

### 3-amino-N-(cyclobutylmethyl)pyridine-2-carboxamide

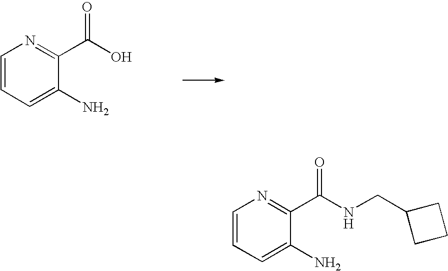

The title compound was prepared by applying general procedure 5 to 3-aminopyridine-2-carboxylic acid. The reaction mixture was subjected to aqueous work-up (NaHCO₃) and the organic layer was separated and dried. The crude product was purified using silica based chromatography with an eluent system containing EtOAc/heptane (1:1) to afford the title compound (28%).

¹H NMR (CDCl₃, 400 MHz) δ (ppm) 1.71 (m, 2H), 1.86-1.96 (m, 2H), 2.06-2.15 (m, 2H), 2.53-2.65 (m, 1H), 3.41-3.46 (m, 2H), 5.95 (bs, 2H), 6.98 (d, 1H), 7.14 (dd, 1H), 7.85 (d, 1H), 8.09 (bs, 1H).

### Example 52

### N-{2-[(cyclobutylmethyl)carbamoyl]pyridin-3-yl}-1-methyl-1H-indole-2-carboxamide

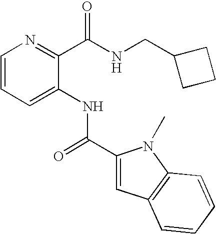

The title compound was prepared by applying general procedure 3 to 3-amino-N-(cyclobutylmethyl)pyridine-2-carboxamide. The acid chloride was prepared according general procedure 2 starting with 1-methyl-1H-indole-2-carboxylic acid. The reaction was subjected to aqueous work-up (NaHCO₃) and purification was accomplished using silica based chromatography (EtOAc/heptane 1:2) to afford the title compound (39%) as a colorless solid.

¹H NMR (CDCl₃, 400 MHz) δ (ppm) 1.74-1.87 (m, 2H), 1.89-1.99 (m, 2H), 2.10-2.20 (m, 2H), 2.58-2.72 (m, 1H), 4.13 (s, 3H), 7.14-7.19 (m, 1H), 7.32-7.47 (m, 4H), 7.74 (d, 1H), 8.23 (dd, 1H), 8.47 (bs, 1H), 9.23 (dd, 1H), 13.15 (bs, 1H); MS (ESI) (M+H)⁺ 363.1, MS (ESI) (M−H)⁻ 361.0.

### Example 53

### N-{2-[(cyclobutylmethyl)carbamoyl]pyridin-3-yl}-1H-indole-3-carboxamide

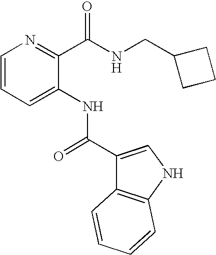

The title compound was prepared by applying general procedure 3 to 3-amino-N-(cyclobutylmethyl)pyridine-2-carboxamide. The acid chloride was prepared from 1H-indole-3-carboxylic acid following general procedure 2. Purification was made by running reversed phase HPLC (CH₃CN/water with CH₃COOH as buffer). The fractions containing the title compound were evaporated under reduced pressure and the remaining water phase was made basic with NaHCO₃ (s) and extracted with CH₂Cl₂. The organic phase was dried and concentrated to afford the title compound (23%) as a colorless solid.

¹H NMR (CDCl₃, 400 MHz) δ (ppm) 1.74-1.85 (m, 2H), 1.88-1.98 (m, 2H), 2.09-2.19 (m, 2H), 2.57-2.69 (m, 1H), 3.48-3.52 (m, 2H), 7.27-7.33 (m, 2H), 7.40-7.47 (m, 2H), 8.00 (d, 1H), 8.19 (dd, 1H), 8.41-8.45 (m, 1H), 8.50 (bs, 1H), 8.78 (bs, 1H), 9.32 (dd, 1H), 12.82 (bs, 1H); MS (ESI) (M+H)⁺ 349.1, MS (ESI) (M−H)⁻ 347.0.

### Example 54

### N-{2-[(cyclobutylmethyl)carbamoyl]-4-methoxyphenyl}quinoline-4-carboxamide

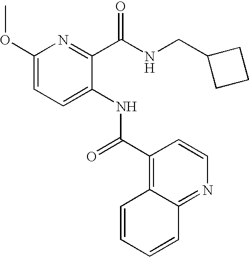

### Example 54A

### N-[2-{(cyclobutylmethyl)carbamoyl]-4-methoxyphenyl}quinoline-4-carboxamide

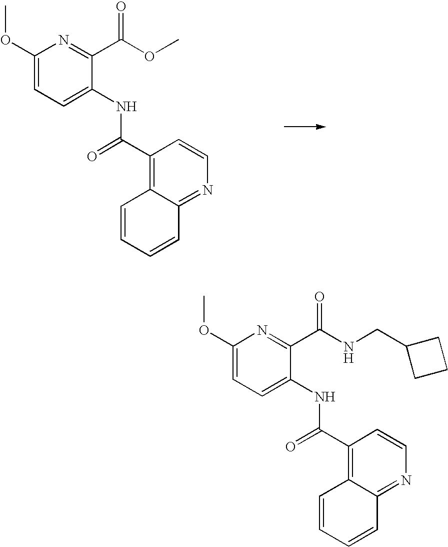

The title compound was prepared by applying general procedure 4 to methyl-5-methoxy-2-[(quinolin-4-ylcarbonyl)amino]benzoate, obtained from Example 54B, and by using cyclobutylmethylamine. The reaction mixture was directly chromatographed on a silica based system with EtOAc/heptane (1:4→1:1) as eluent to afford the title compound (80%) as a colorless solid.

¹H NMR (CDCl₃, 400 MHz) δ (ppm) 1.69-1.81 (m, 2H), 1.85-1.98 (m, 2H), 2.05-2.15 (m, 2H), 2.52-2.65 (m, 1H), 3.39-3.45 (m, 2H), 3.95 (s, 3H), 7.03 (d, 1H), 7.59-7.65 (m, 1H), 7.71 (d, 1H), 7.74-7.80 (m, 1H), 8.12-8.20 (m, 2H), 8.47 (d, 1H), 9.04 (d, 1H), 9.30 (d, 1H), 12.82 (bs, 1H); MS (ESI) (M+H)⁺ 391.2, MS (ESI) (M−H)⁻ 389.1.

### Example 54B

### Methyl-5-methoxy-2-[(quinolin-4-ylcarbonyl)amino]benzoate

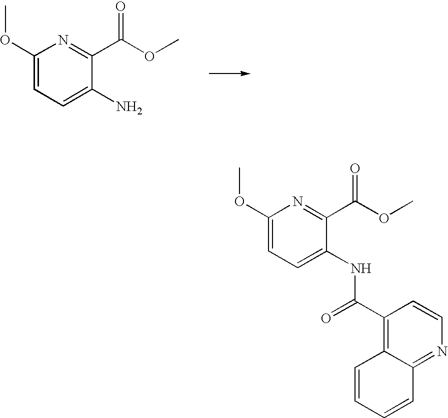

The title compound was prepared by applying general procedure 3 to methyl-3-amino-6-methoxypyridine-2-carboxylate obtained by applying general procedure 6b on 3-Amino-6-methoxy-pyridine-2-carboxylic acid from Example 1E. The acid chloride was prepared by applying general procedure 2 to quinoline-4-carboxylic acid. The reaction mixture was subjected to aqueous work-up (NaHCO₃) and the organic phase was separated and dried. The crude product was purified using silica based chromatography with an eluent system containing CH₂Cl₂/EtOAc (1:0→4:1) to afford the title compound (39%) as a colorless solid.

¹H NMR (CDCl₃, 400 MHz) δ (ppm) 3.96 (s, 3H), 4.01 (s, 3H), 7.10 (d, 1H), 7.65 (ddd, 1H), 7.69 (d, 1H), 7.80 (ddd, 1H), 8.20 (d, 1H), 8.45 (d, 1H), 9.07 (d, 1H), 9.25 (d, 1H), 11.55 (bs, 1H); MS (ESI) (M+H)⁺ 338.1.

### Example 55

### N-{2-[(cyclobutylmethyl)carbamoyl]-6-methoxypyridin-3-yl}-1-methyl-1H-indazole-3-carboxamide

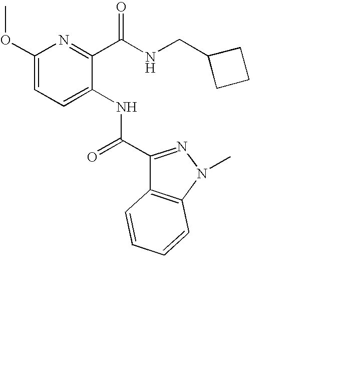

### Example 55A

### N-{2-[(cyclobutylmethyl)carbamoyl]-6-methoxypyridin-3-yl}-1-methyl-1H-indazole-3-carboxamide

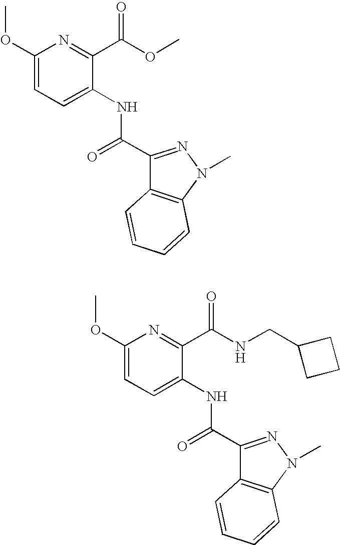

The title compound was prepared by applying general procedure 4 to methyl-6-methoxy-3-{[(1-methyl-1H-indazol-3-yl)carbonyl]amino}pyridine-2-carboxylate (obtained from 55B) and by using cyclobutylmethylamine. After 6 h at 90° C. the reaction mixture was also heated to 150° C. using microwave irradiation for 30 minutes. Purification on a silica based system was run with an isocratic system, EtOAc/heptane (2:3), to afford the title compound (12%) as a colorless solid.

¹H NMR (CDCl₃, 400 MHz) δ (ppm) 1.75-1.86 (m, 2H), 1.90-2.01 (m, 2H), 2.09-2.19 (m, 2H), 2.59-2.70 (m, 1H), 3.51-3.57 (m, 2H), 3.94 (s, 3H), 4.22 (s, 3H), 6.97 (d, 1H), 7.29-7.34 (m, 1H), 7.43-7.46 (m, 2H), 8.16 (bs, 1H), 8.41 (d, 1H), 9.32 (d, 1H), 13.07 (bs, 1H); MS (ESI) (M+H)⁺ 394.2, MS (ESI) (M−H)⁻ 392.1.

### Example 55B

### Methyl-6-ethoxy-3-{[(1-methyl-1H-indazol-3-yl)carbonyl]amino}pyridine-2-carboxylate

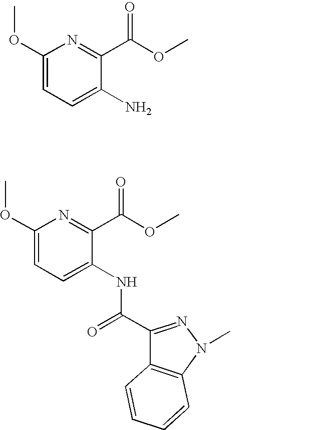

The title compound was prepared by applying general procedure 3 to methyl-3-amino-6-methoxypyridine-2-carboxylate obtained by applying general procedure 6b on 3-Amino-6-methoxy-pyridine-2-carboxylic acid from Example 1E. The acid chloride was prepared by applying general procedure 2 to 1-methyl-1H-indazole-3-carboxylic acid. The reaction was subjected to aqueous work-up (NaHCO₃) and purification was accomplished using silica based chromatography (CH₂Cl₂/EtOAc 1:0-4:1) to afford the title compound (67%) as a colorless solid.

¹H NMR (CDCl₃, 400 MHz) δ (ppm) 3.99 (s, 3H), 4.06 (s, 3H), 4.22 (s, 3H), 7.04 (d, 1H), 7.31-7.36 (m, 1H), 7.45-7.48 (m, 2H), 8.41 (d, 1H), 9.27 (d, 1H), 12.06 (bs, 1H); MS (ESI) (M+H)⁺ 341.1.

### Example 56

### 3-[(1-benzothien-3-yl-carbonyl)amino]-N-(tetrahydro-2H-pyran-4-ylmethyl)pyridine-2-carboxamide

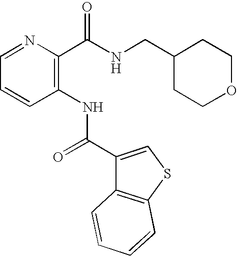

### Example 56A

### 3-[(1-benzothien-3-ylcarbonyl)amino]-N-(tetrahydro-2H-pyran-4-ylmethyl)pyridine-2-carboxamide

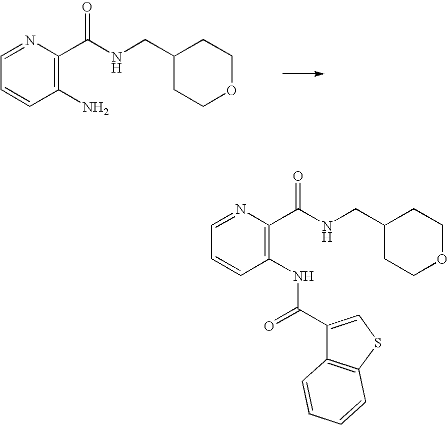

Following the general procedure 6, using benzothiophene-3-carboxylic acid chloride (prepared from benzothiophene-3-carboxylic acid (178 mg, 1 mmol) using general procedure 2) and 3-amino-N-(tetrahydro-2H-pyran-4-ylmethyl)pyridine-2-carboxamide (74 mg, 0.3 mmol), obtained from Example 566B, provided the title compound (102 mg, 86% yield) after purification.

¹H NMR (400 MHz, CD₃OD) δ (ppm) 1.27-1.41 (m, 2H), 1.67 (bd, 2H), 1.86-1.98 (m, 1H), 3.31 (d, 2H), 3.38 (dd, 2H), 3.93 (dd, 2H), 7.39-7.50 (m, 2H), 7.55 (dd, 1H), 7.95 (d, 1H), 8.31 (dd, 1H), 8.40 (s, 1H), 8.58 (d, 1H), 9.18 (dd, 1H); MS (ESI) (M+H)⁺: 396

### Example 56B

### 3-amino-N-(tetrahydro-2H-pyran-4-ylmethyl)pyridine-2-carboxamide

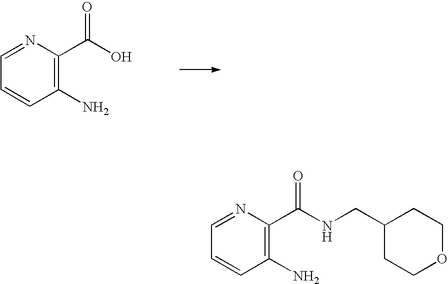

Following the general procedure 5 using 3-aminopyridine-2-carboxylic acid (6.7 g, 48.6 mmol) and 1-(4-tetrahydropyranyl)-methylamine (5.6 g, 48.6 mmol) provided the title compound (7.4 g, 65% yield) after recrystallisation from hexane.

¹H NMR (400 MHz, CDCl₃) δ (ppm) 1.29-1.42 (m, 2H), 1.65 (bd, 2H), 1.75-1.89 (m, 1H), 3.28 (t, 2H), 3.34 (dd, 2H), 3.95 (dd, 2H), 5.94 (bs, 2H), 6.96 (d, 1H), 7.11 (dd, 1H), 7.80 (d, 1H), 8.20 (bs, 1H); MS (ESI) (M+H)⁺: 236

### Example 57

### 3-[(5,6,7,8-tetrahydronaphthalen-1-ylcarbonyl)amino]-N-(tetrahydro-2H-pyran-4-ylmethyl)pyridine-2-carboxamide

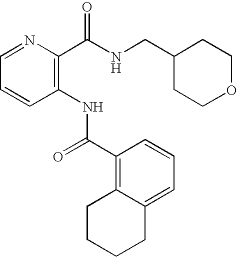

Following the general procedure 6, using 5,6,7,8-tetrahydronaphtalen-1-carboxylic acid chloride (prepared from 5,6,7,8-tetrahydronaphtalen-1-carboxylic acid (176 mg, 1 mmol) using general procedure 2) and 3-amino-N-(tetrahydro-2H-pyran-4-ylmethyl)pyridine-2-carboxamide, obtained from Example 56B, (74 mg, 0.3 mmol) provided the title compound (57 mg, 48.5% yield) after purification.

¹H NMR (400 MHz, CD₃OD) δ (ppm) 1.21-1.34 (m, 2H), 1.60 (bd, 2H), 1.77-1.88 (m, 1H partly hidden behind water peak), 2.72-2.83 (m, 4H), 2.90 (bt, 2H), 3.00 (bt, 2H), 3.21 (d, 2H), 3.32 (dd, 2H), 3.88 (dd, 2H), 7.07 (t, 1H), 7.13-7.20 (m, 2H), 7.31-7.36 (m, 1H), 7.50 (dd, 1H), 7.59 (d, 1H), 8.27 (d, 1H), 9.12 (d, 1H); MS (ESI) (M+H)⁺: 394

### Example 58

### N-{2-[(tetrahydro-2H-pyran-4-ylmethyl)carbamoyl]pyridin-3-yl}-1H-indazole-3-carboxamide

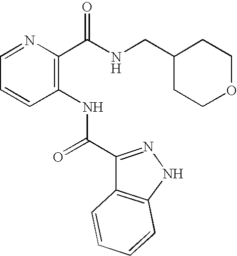

Following the general procedure 6, using 1H-indazole-3-carboxylic acid chloride (prepared from 1H-indazole-3-carboxylic acid (162 mg, 1 mmol) using general procedure 2) and 3-amino-N-(tetrahydro-2H-pyran-4-ylmethyl)pyridine-2-carboxamide, obtained from Example 56B, (74 mg, 0.3 mmol) provided the title compound (7 mg, 6.2% yield) after purification.

¹H NMR (400 MHz, CD₃OD) δ (ppm) 1.29-1.42 (m, 2H), 1.69 (bd, 2H), 1.86-2.01 (m, 1H), 3.33 (d, 2H), 3.39 (dd, 2H), 3.93 (dd, 2H), 7.28 (t, 1H), 7.43 (t, 1H), 7.55 (dd, 1H), 8.25-8.33 (s+t, 2H), 9.29 (d, 1H); MS (ESI) (M+H)⁺: 380

### Example 59

N-{2-[(tetrahydro-2H-pyran-4-ylmethyl)carbamoyl]pyridin-3-yl}-1H-indole-3-carboxamide

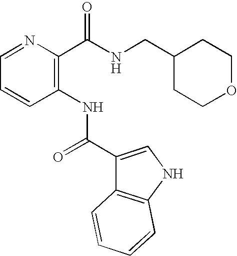

Following the general procedure 6, using 1H-indole-3-carboxylic acid chloride (prepared from 1H-indole-3-carboxylic acid (161 mg, 1 mmol) using general procedure 2) and 3-amino-N-(tetrahydro-2H-pyran-4-ylmethyl)pyridine-2-carboxamide, obtained from Example 56B (74 mg, 0.3 mmol) provided the title compound (89 mg, 78.5% yield) after purification.

¹H NMR (400 MHz, CD₃OD) δ (ppm) 1.34-1.48 (m, 2H), 1.70 (d, 2H), 1.83-1.96 (m, 1H), 3.33-3.44 (d+dd, 4H), 3.99 (dd, 2H), 7.28 (t, 2H), 7.37-7.49 (m, 2H), 7.99 (s, 1H), 8.17 (d, 1H), 8.40 (d, 1H), 8.59-8.71 (2s, 2H), 9.32 (d, 1H), 12.74 (s, 1H); MS (ESI) (M+H)⁺: 379

### Example 60

### 1-methyl-N-{2-[(tetrahydro-2H-pyran-4-ylmethyl)carbamoyl]pyridin-3-yl}-1H-indole-3-carboxamide

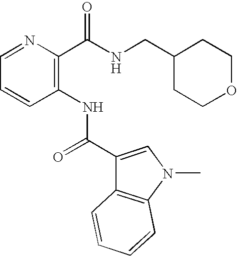

Following the general procedure 6, using N-methyl-indole-3-carboxylic acid chloride (prepared from N-methyl-indole-3-carboxylic acid (175 mg, 1 mmol) using general procedure 2) and 3-amino-N-(tetrahydro-2H-pyran-4-ylmethyl)pyridine-2-carboxamide, obtained from Example 56B (125 mg, 0.5 mmol) provided the title compound (121 mg, 62% yield) after purification.

¹H: NMR (400 MHz, CD₃OD) δ (ppm) 1.30-1.43 (m, 2H), 1.69 (bd, 2H), 1.85-1.98 (m, 1H), 3.33 (d, 2H), 3.39 (dd, 2H), 3.89 (s, 3H), 3.94 (dd, 2H), 7.19-7.31 (2t, 2H), 7.43 (d, 1H), 7.47 (dd, 1H), 7.92 (s, 1H), 8.20-8.28 (s+d, 2H), 9.14 (d, 1H); MS (ESI) (M+H)⁺: 393

### Example 61

### N-{2-[(tetrahydro-2H-pyran-4-ylmethyl)carbamoyl]pyridin-3-yl}-1,3-benzothiazole-6-carboxamide

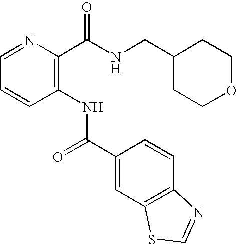

Following the general procedure 6, using 1,3-benzothiazole-6-carboxylic acid chloride (prepared from 1,3-benzothiazole-6-carboxylic acid (179 mg, 1 mmol) using general procedure 2) and 3-amino-N-(tetrahydro-2H-pyran-4-ylmethyl)pyridine-2-carboxamide, obtained from Example 56B (74 mg, 0.3 mmol) provided the title compound (15.4 mg, 12.9% yield) after purification.

¹H NMR (400 MHz, CDCl₃) δ (ppm) 1.35-1.49 (m, 2H), 1.71 (bd, 2H), 1.84-1.98 (m, 1H), 3.34-3.44 (m, 4H), 3.99 (dd, 2H), 7.50 (dd, 1H), 8.17-8.28 (m, 3H), 8.67 (bt, 1H), 8.71 (s, 1H), 9.14 (s, 1H), 9.34 (d, 1H), 13.30 (s, 1H); MS (ESI) (M+H)⁺: 397

### Example 62

### N-{2-[(tetrahydro-2H-pyran-4-ylmethyl)carbamoyl]pyridin-3-yl}-1,6-naphthyridine-5-carboxamide

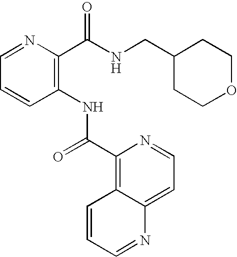

Following the general procedure 6, using 1,6-naphtpyridine-5-carboxylic acid chloride (prepared from 1,6-naphtpyridine-5-carboxylic acid (174 mg, 1 mmol) using general procedure 2) and 3-amino-N-(tetrahydro-2H-pyran-4-ylmethyl)pyridine-2-carboxamide, obtained from Example 56B (125 mg, 0.5 mmol) provided the title compound (57 mg, 29% yield) after purification.

¹H NMR (400 MHz, CD₃OD) δ (ppm) 1.30-1.43 (m, 2H), 1.70 (bd, 2H), 1.86-1.99 (m, 1H), 3.31-3.44 (2t, 4H), 3.93 (bdd, 2H), 7.55 (dd, 1H), 7.71 (dd, 1H), 8.09 (d, 1H), 8.32 (d, 1H), 8.91 (d, 1H), 9.05-9.14 (m, 2H), 9.32 (d, 1H), 9.89 (d, 1H), 13.88 (s, 1H); MS (ESI) (M+H)⁺: 392

### Example 63

### 3-{[(6-fluoro-4H-1,3-benzodioxin-8-yl)carbonyl]amino}-N-(tetrahydro-2H-pyran-4-ylmethyl)pyridine-2-carboxamide

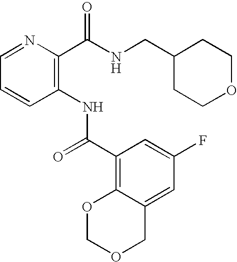

Following the general procedure 6b, using 6-fluoro-4H-1,3-benzodioxin-8-carboxylic acid chloride (prepared from 6-fluoro-4H-1,3-benzodioxin-8-carboxylic acid (99 mg, 0.5 mmol) using general procedure 2) and 3-amino-N-(tetrahydro-2H-pyran-4-ylmethyl)pyridine-2-carboxamide, obtained from Example 56B, (118 mg, 0.5 mmol) provided the title compound (143 mg, 69% yield) after purification.

¹H NMR (400 MHz, CD₃OD) δ (ppm) 1.27-1.40 (m, 2H), 1.62-1.71 (bd, 2H), 1.82-1.94 (m, 1H), 3.28-3.32 (d, 2H partly hidden in solvent), 3.38 (dt, 2H), 3.88-3.96 (bd, 2H), 4.97 (s, 2H), 5.46 (s, 2H), 7.05 (dd, 1H), 7.51 (dd, 1H), 7.57 (dd, 1H), 8.31 (d, 1H), 9.20 (d, 1H); MS (ESI) (M+H)⁺: 416

### Example 64

### N-{2-[(cyclobutylmethyl)carbamoyl]pyridin-3-yl}-1H-indazole-3-carboxamide

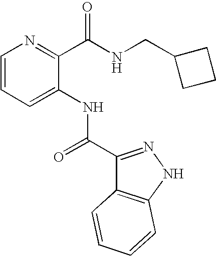

Following the general procedure 6, using 1H-indazole-3-carboxylic acid chloride (prepared from 1H-indazole-3-carboxylic acid (1.19 g, 7.3 mmol) using general procedure 2) and 3-amino-N-(cyclobutylmethyl)pyridine-2-carboxamide, obtained from Example 51B, (0.5 g, 2.4 mmol) provided the title compound (20 mg, 0.8% yield) after purification.

¹H NMR (400 MHz, CDCl₃) δ (ppm) 1.68-1.80 (m, 2H), 1.81-1.94 (m, 2H), 2.03-2.13 (m, 2H), 2.53-2.67 (m, 1H), 3.51 (dd, 2H), 7.29 (t, 1H), 7.39 (t, 1H), 7.47 (dd, 1H), 7.55 (d, 1H), 8.24 (dd, 1H), 8.41 (d, 1H), 8.50 (bd, 1H), 9.30 (dd, 1H), 11.37 (s, 1H), 13.26 (s, 1H); MS (ESI) (M+H)⁺: 350

### Example 65

### 3-[(4-{[(5-methylisoxazol-3-yl)methoxy]methyl}-1-naphthoyl)amino]-N-(tetrahydro-2H-pyran-4-ylmethyl)pyridine-2-carboxamide

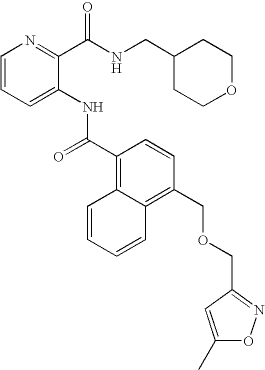

3-{[4-(bromomethyl)-1-naphthoyl]amino}-N-(tetrahydro-2H-pyran-4-ylmethyl)pyridine-2-carboxamide, prepared according to Example 129 and 130 of document WO 2005/115986 (20 mg, 0.042 mmol), was added to a solution of NaH (1.5 mg, 0.06 mmol) and (5-s methylisoxazol-3-yl)methanol (7 mg, 0.06 mmol) in acetonitrile (0.5 ml). The reaction was stirred for 2 h under nitrogen in room temperature. The mixture was diluted with water and DCM, dried over MgSO₄, filtered and concentrated under reduced pressure. The crude product was purified by preparative HPLC to give 1.3 mg (6.1%) of 3-[(4-{[(5-methylisoxazol-3-yl)methoxy]methyl}-1-naphthoyl)amino]-N-(tetrahydro-2H-pyran-4-ylmethyl)pyridine-2-carboxamide.

¹H NMR (400 MHz, CDCl₃) δ (ppm) 1.32-1.44 (m, 1H), 1.62-1.90 (m, 2H), 2.45 (s, 3H), 2.61 (s, 2H), 3.28-3.40 (m, 4H), 3.95-4.02 (m, 2H), 4.68 (s, 2H), 5.04 (s; 2H), 7.50-7.63 (m, 4H), 7.84-7.89 (m, 1H), 8.09-8.15 (m, 2H), 8.25-8.30 (m, 1H), 8.52-8.60 (m, 1H), 9.38-9.43 (m, 1H).

1. A compound of formula (I) 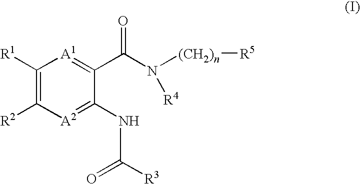
wherein:
at least one of A¹ and A² is N and if both are not N, then the other is CH;
R¹ is selected from hydrogen, cyano, halogen, hydroxy, NR⁶R⁷, C₂₋₆alkenyl, C₂₋₆alkynyl, C₁₋₉alkyl, C₃₋₆cycloalkyl and C₁₋₆haloalkoxy, wherein said C₂₋₆alkenyl, C₂₋₆alkynyl, C₁₋₉alkyl, C₃₋₆cycloalkyl or C₁₋₆haloalkoxy is optionally substituted by hydroxy, NR^{6a}R^{7a}, C₃₋₆cycloalkyl, aryl and heteroaryl;
R² is selected from hydrogen, cyano, halogen, hydroxy, NR⁶R⁷, C₂₋₆alkenyl, C₂₋₆alkynyl, C₁₋₆alkyl, C₃₋₆cycloalkyl and C₁₋₆haloalkoxy, wherein said C₂₋₆alkenyl, C₂₋₆alkynyl, C₁₋₆alkyl, C₃₋₆cycloalkyl or C₁₋₆haloalkoxy is optionally substituted by hydroxy, NR^{6a}R^{7a}, C₃₋₆cycloalkyl, aryl and heteroaryl;
R³ is selected from 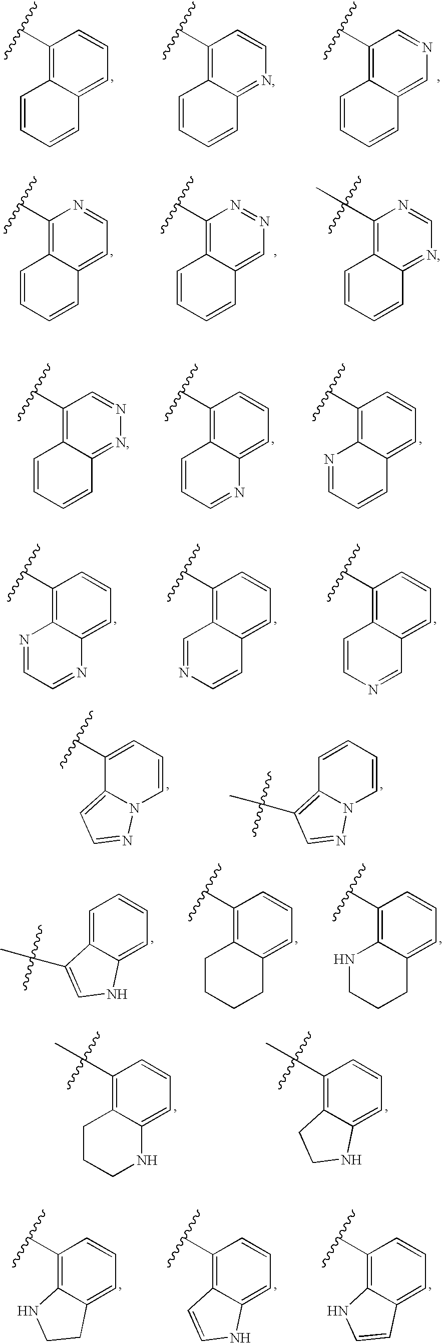 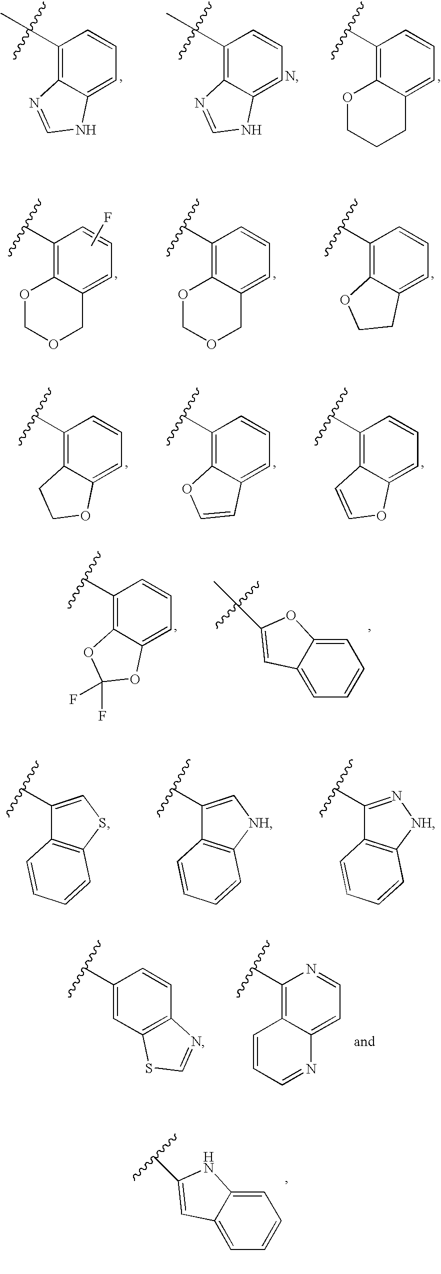
and wherein R³ is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl, wherein said C₁₋₆alkyl, C₃₋₆cycloalkyl, aryl or heteroaryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl, heteroaryl or a saturated ring system consisting of 4 to 7 atoms selected from C, N and O, and wherein said C₁₋₆alkyl, C₃₋₆cycloalkyl, aryl, heteroaryl or ring system is optionally substituted by C₁₋₄alkyl, and wherein said C₁₋₄alkyl is optionally substituted by NR⁶R⁷, aryl, hydroxy or C₁₋₄alkoxy;
R⁴ is selected from hydrogen and C₁₋₆alkyl;
R⁵ is selected from C₁₋₆alkyl, C₃₋₆cycloalkyl, C₁₋₆alkoxy, C₁₋₆haloalkoxy, heteroaryl and aryl, wherein said C₁₋₆alkyl, C₃₋₆cycloalkyl, heteroaryl or aryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
n is selected from 0, 1, 2, 3, 4 and 5;
or R⁴ and R⁵ together form a saturated, unsaturated or partly saturated ring system consisting of 3 to 7 atoms selected from C, O and N;
or R⁴ and R⁵ together form a saturated, unsaturated or partly saturated condensed ring system consisting of 7 to 13 atoms selected from C, O and N;
wherein said ring system is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl, and wherein said C₁₋₆alkyl, C₃₋₆cycloalkyl, aryl or heteroaryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
R⁶, R^{6a}, R⁷ and R^{7a} are each and independently selected from hydrogen, C₁₋₆alkyl, C₃₋₆cycloalkyl, C₁₋₆alkoxy, C₁₋₆haloalkoxy, C₂₋₆alkenyl, C₂₋₆alkynyl, aryl and heteroaryl; or
R^{6a} and R^{7a} may together form a saturated ring system consisting of 4 to 7 atoms selected from C, O and N, which ring system is optionally substituted with C₁₋₆alkyl, C₁₋₆alkoxy, halogen or hydroxy;
wherein one or more carbon atom(s) of each alkyl or cycloalkyl group as defined for R¹, may be substituted for O, NH, C(O), SO or SO₂, wherein none of the N or O is in a position adjacent to any other O or N and wherein none of the SO or SO₂ is in a position adjacent to any other SO or SO₂;
wherein one or more carbon atom(s) of each alkyl or cycloalkyl group as defined for R², R³, R⁴, R⁵, R⁶, R^{6a}, R⁷ and R^{7a} may be substituted for O, NH, C(O) or SO₂, wherein none of the N or O is in a position adjacent to any other O or N;
wherein one or more carbon atom(s) of each alkyl or cycloalkyl group as defined for R¹, R², R³, R⁴, R⁵, R⁶, R^{6a}, R⁷ and R^{7a} may be substituted by fluoro; and
with the proviso that R¹ is not hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl or NR⁶R⁷; at the same time as R² is hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl or NR⁶R⁷;
unless R³ is substituted by a C₁₋₄ alkyl, which C₁₋₄alkyl is substituted by a heteroaryl, C₃₋₆cycloalkyl, aryl or a saturated ring system consisting of 4 to 7 atoms selected from C, O and N, wherein said heteroaryl, C₃₋₆cycloalkyl or aryl is further substituted by C₁₋₄alkyl or halogen, wherein said C₁₋₄alkyl is optionally substituted by NR⁶R⁷, aryl, hydroxy or C₁₋₄alkoxy, and wherein said ring system is optionally substituted by C₁₋₄alkyl, wherein said C₁₋₄ alkyl is optionally substituted by NR⁶R⁷, aryl, hydroxy or C₁₋₄alkoxy;
or unless R³ is selected from: 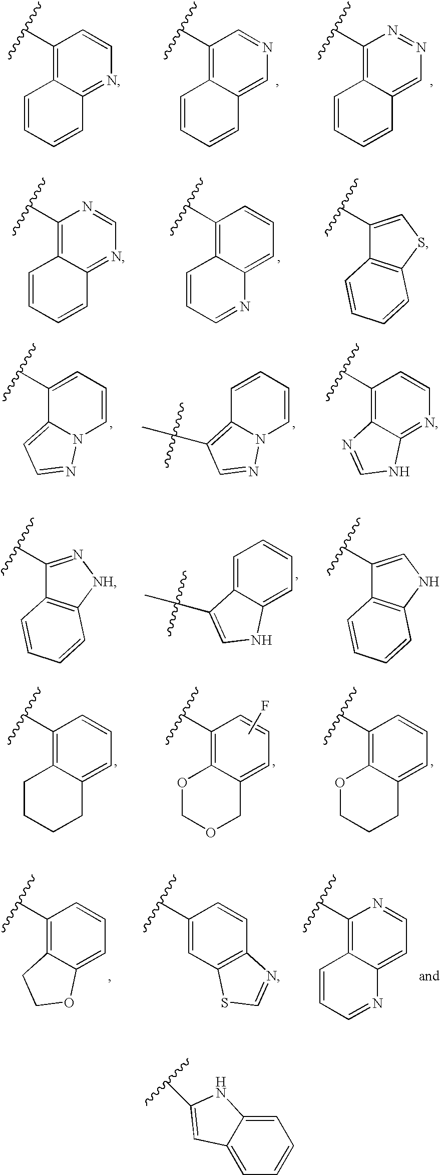
optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
or a pharmaceutically acceptable salt thereof, or diastereomers, or enantiomers, or mixtures thereof.

2. A compound of formula (I) 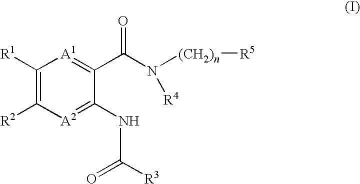
wherein:
at least one of A¹ and A² is N and if both are not N, then the other is CH;
R¹ is selected from hydrogen, cyano, halogen, hydroxy, NR⁶R⁷, C₂₋₆alkenyl, C₂₋₆alkynyl, C₁₋₆alkyl, C₃₋₆cycloalkyl and C₁₋₆haloalkoxy, wherein said C₂₋₆alkenyl, C₂₋₆alkynyl, C₁₋₆alkyl, C₃₋₆cycloalkyl or C₁₋₆haloalkoxy is optionally substituted by hydroxy, NR^{6a}R^{7a}, C₃₋₆cycloalkyl, aryl or heteroaryl;
R² is selected from hydrogen, cyano, halogen, hydroxy, NR⁶R⁷, C₂₋₆alkenyl, C₂₋₆alkynyl, C₁₋₆alkyl, C₃₋₆cycloalkyl and C₁₋₆haloalkoxy, wherein said C₂₋₆alkenyl, C₂₋₆alkynyl, C₁₋₆alkyl, C₃₋₆cycloalkyl or C₁₋₆haloalkoxy is optionally substituted by hydroxy, NR^{6a}R^{7a}, C₃₋₆cycloalkyl, aryl or heteroaryl;
R³ is selected from 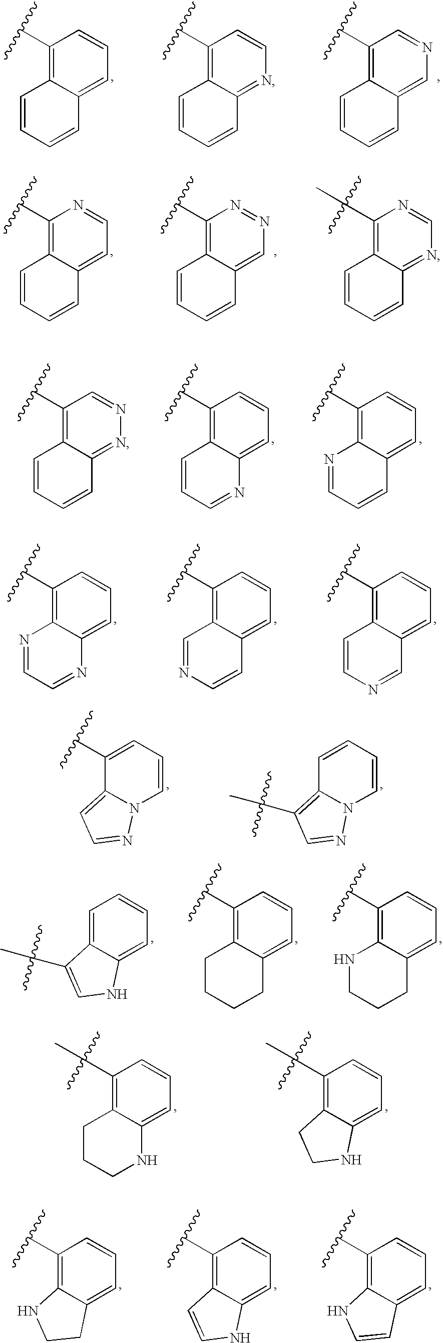 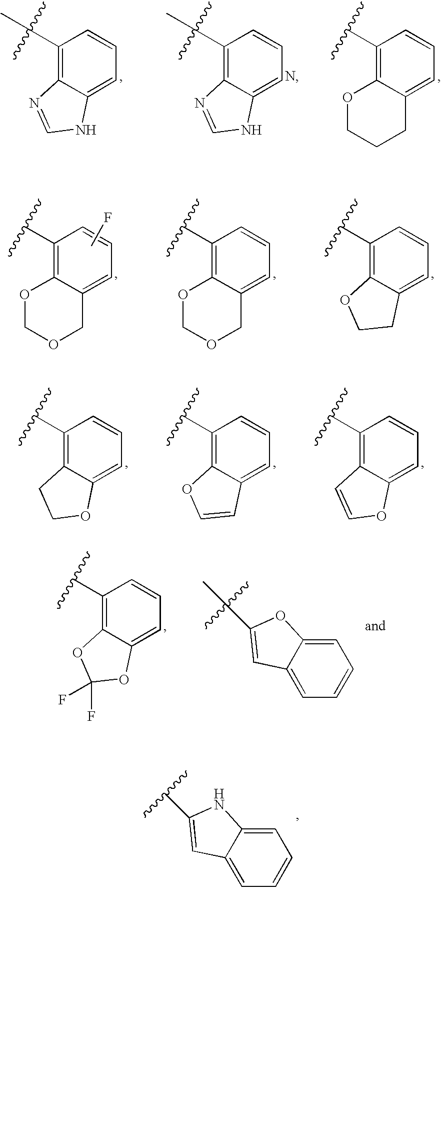
and wherein R³ is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl, wherein said C₁₋₆alkyl, C₃₋₆cycloalkyl, aryl or heteroaryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl; and wherein said C₁₋₆alkyl, C₃₋₆cycloalkyl, aryl or heteroaryl is optionally substituted by C₁₋₄alkyl and wherein said C₁₋₄alkyl is optionally substituted by NR⁶R⁷, aryl, hydroxy or C₁₋₄alkoxy;
R⁴ is selected from hydrogen and C₁₋₆alkyl;
R⁵ is selected from C₁₋₆alkyl, C₃₋₆cycloalkyl, C₁₋₆alkoxy, C₁₋₆haloalkoxy, heteroaryl and aryl, wherein said C₁₋₆alkyl, C₃₋₆cycloalkyl, heteroaryl or aryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
n is selected from 0, 1, 2, 3, 4 and 5;
or R⁴ and R⁵ together form a saturated, unsaturated or partly saturated ring system consisting of 3 to 7 atoms selected from C, O and N;
or R⁴ and R⁵ together form a saturated, unsaturated or partly saturated condensed ring system consisting of 7 to 13 atoms selected from C, O and N;
wherein said ring system is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl, and wherein said C₁₋₆alkyl, C₃₋₆cycloalkyl, aryl or heteroaryl is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
R⁶, R^{6a}, R⁷ and R^{7a} are each and independently selected from hydrogen, C₁₋₆alkyl, C₃₋₆cycloalkyl, C₁₋₆alkoxy, C₁₋₆haloalkoxy, C₂₋₆alkenyl, C₂₋₆alkynyl, aryl and heteroaryl;
wherein one or more carbon atom(s) of each alkyl or cycloalkyl group as defined for R¹, R², R³, R⁴, R⁵, R⁶, R^{6a}, R⁷ and R^{7a} may be substituted for O, NH, C(O) or SO₂;
wherein one or more carbon atom(s) of each alkyl or cycloalkyl group as defined for R¹, R², R³, R⁴, R⁶, R^{6a}, R⁷ and R^{7a} may be substituted by fluoro; and
with the proviso that R¹ is not hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl or NR⁶R⁷; at the same time as R² is hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl or NR⁶R⁷; unless R³ is substituted by a C₁₋₄ alkyl, which C₁₋₄alkyl is substituted by a heteroaryl, C₃₋₆cycloalkyl or aryl that is further substituted by C₁₋₄alkyl wherein said C₁₋₄alkyl is optionally substituted by NR⁶R⁷, aryl, hydroxy or C₁₋₄alkoxy;
or unless R³ is selected from: 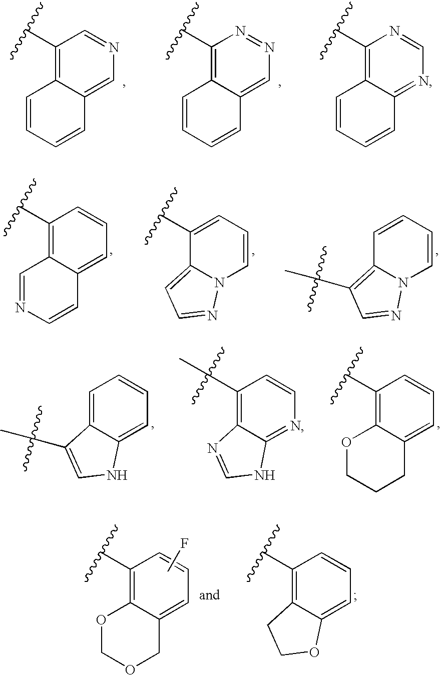
or a pharmaceutically acceptable salt thereof, or diastereomers, or enantiomers, or mixtures thereof.

3. A compound according to claim 1, wherein A¹ and A² are N.

4. A compound according to claim 1, wherein A¹ is N and A² is CH.

5. A compound according to claim 1, wherein R⁴ is hydrogen.

6. A compound according to claim 1, wherein n is 1.

7. A compound according to claim 1, wherein R² is hydrogen.

8. A compound according to claim 1, wherein R² is C₁₋₆alkyl, optionally substituted by hydroxy, NR^{6a}R^{7a}, C₃₋₆cycloalkyl, aryl or heteroaryl.

9. A compound according to claim 1, wherein R¹ is hydrogen.

10. A compound according to claim 1, wherein R¹ is selected from cyano, halogen, NR⁶R⁷, C₁₋₉alkyl, C₃₋₆cycloalkyl and C₁₋₆haloalkoxy, wherein said C₁₋₉alkyl, C₃₋₆cycloalkyl or C₁₋₆haloalkoxy is optionally substituted by hydroxy, NR^{6a}R^{7a}, C₃₋₆cycloalkyl, aryl or heteroaryl.

11-19. (canceled)

20. A compound according to claim 1, wherein R⁵ is C₃₋₆cycloalkyl.

21-22. (canceled)

23. A compound according to claim 1, wherein R³ is selected from 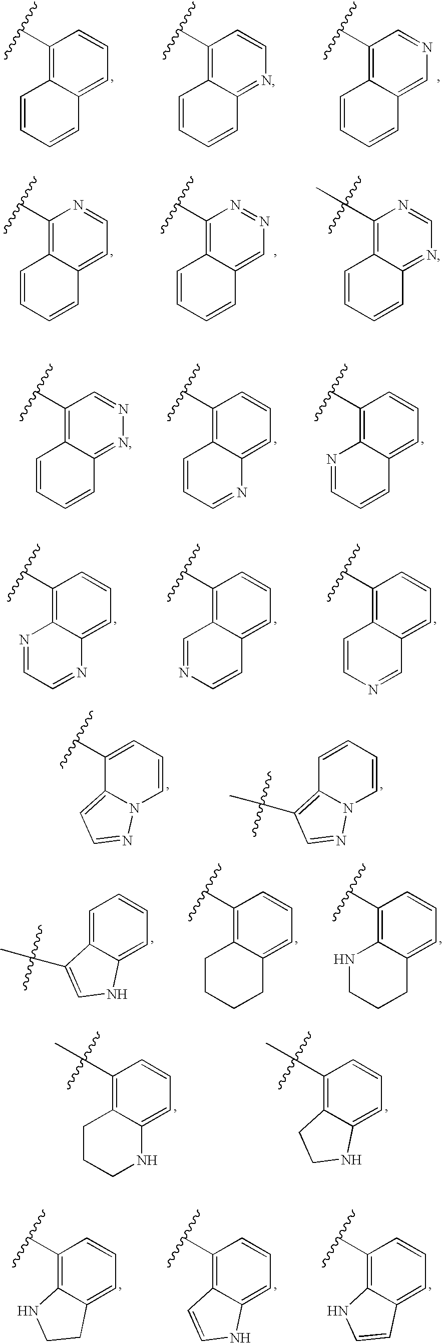 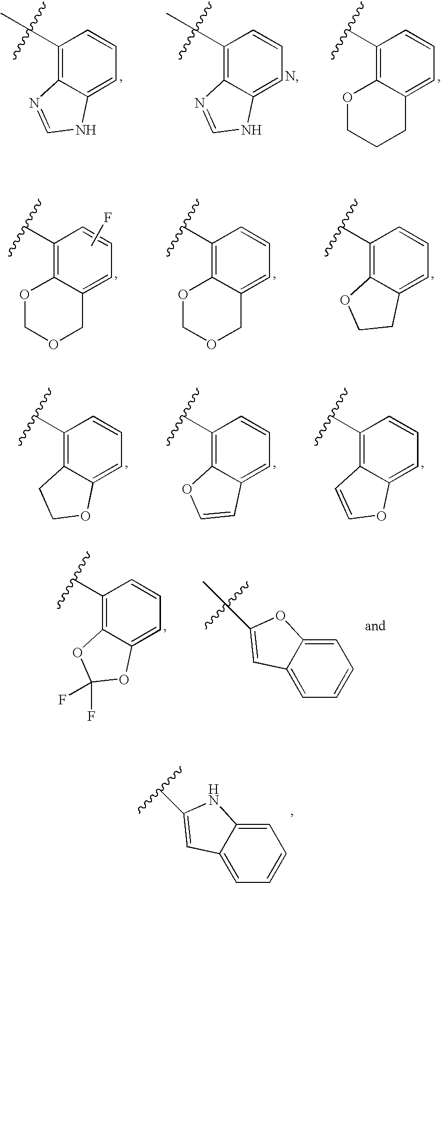
and wherein R³ is optionally substituted by halogen, C₁₋₆alkyl, C₃₋₆cycloalkyl, C₁₋₆alkoxy or C₁₋₆haloalkoxy, wherein said C₁₋₆alkyl or C₃₋₆cycloalkyl is optionally substituted by halogen, NR⁶R⁷, C₁₋₆alkyl, C₃₋₆cycloalkyl, C₁₋₆alkoxy, aryl, heteroaryl or a saturated ring system consisting of 4 to 7 atoms selected from C, N and O and wherein said C₁₋₆alkyl, C₃₋₆cycloalkyl, aryl, heteroaryl or ring system is optionally substituted by C₁₋₄alkyl and wherein said C₁₋₄alkyl is optionally substituted by NR⁶R⁷, aryl, hydroxy or C₁₋₄alkoxy.

24-31. (canceled)

32. A compound according to claim 1, wherein
R¹ is hydrogen or C₁₋₉alkyl, wherein said C₁₋₉alkyl is optionally substituted by hydroxy, NR^{6a}R^{7a}, C₃₋₆cycloalkyl, aryl or heteroaryl;
R² is hydrogen or C₁₋₆alkyl, wherein said C₁₋₆alkyl is optionally substituted by hydroxy, NR^{6a}R^{7a}, C₃₋₆cycloalkyl, aryl or heteroaryl;
R³ is selected from 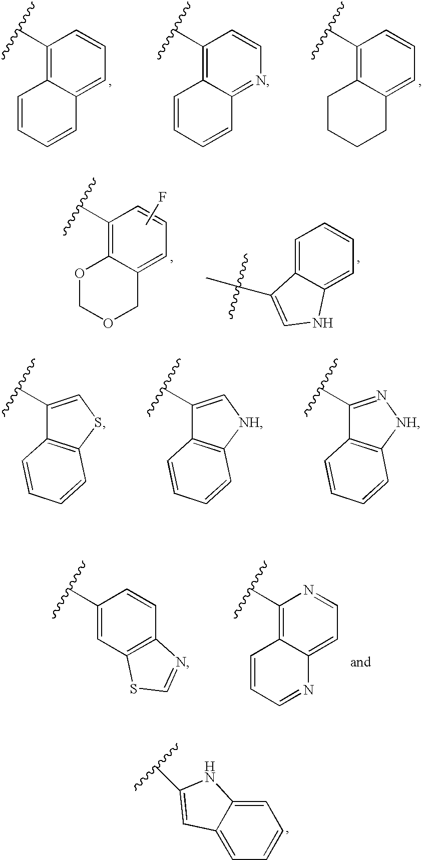
and wherein R³ is substituted by C₁₋₆alkyl, wherein said C₁₋₆alkyl is optionally substituted by heteroaryl or a saturated ring system consisting of 4 to 7 atoms selected from C, N and O, and wherein said heteroaryl or ring system is optionally substituted by C₁₋₄alkyl and wherein said C₁₋₄alkyl is optionally substituted by NR⁶R⁷, aryl, hydroxy or C₁₋₄alkoxy;
R⁴ is hydrogen;
R⁵ is C₃₋₆cycloalkyl;
n is 1;
R⁶, R^{6a}, R⁷ and R^{7a} are each and independently selected from hydrogen and C₁₋₆alkyl; or
R^{6a} and R^{7a} may together form a saturated saturated ring system consisting of 4 to 7 atoms selected from C, O and N; which ring system is optionally substituted with C₁₋₆alkyl, C₁₋₆alkoxy, halogen or hydroxy;
wherein one or more carbon atom(s) of each alkyl or cycloalkyl group as defined for R¹ may be substituted for O, NH, C(O), SO or SO₂ and wherein none of the O or N is in a position adjacent to any other O or N and wherein none of the SO or SO₂ is in a position adjacent to any other SO or SO₂;
wherein one or more carbon atom(s) of each alkyl or cycloalkyl group as defined for R², R³, and R⁵ may be substituted for O, NH, C(O) or SO₂ and wherein none of the O or N is in a position adjacent to any other O or N;
wherein one or more carbon atom(s) of each alkyl or cycloalkyl group as defined for R¹ and R³ may be substituted by fluoro; and
with the proviso that R¹ is not hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl and NR⁶R⁷; at the same time as R² is hydrogen, halogen, cyano, acetylamino, hydroxy, C₁₋₆alkoxy, C₁₋₆alkyl, C₁₋₆haloalkoxy, C₂₋₆alkenyl, C₁₋₆haloalkyl, C₂₋₆haloalkenyl and NR⁶R⁷; unless R³ is substituted by a C₁₋₄ alkyl, which C₁₋₄alkyl is substituted by a heteroaryl, C₃₋₆cycloalkyl, aryl or a saturated ring system consisting of 4 to 7 atoms selected from C, O and N, wherein said heteroaryl, C₃₋₆cycloalkyl or aryl is further substituted by C₁₋₄alkyl or halogen, wherein said C₁₋₄alkyl is optionally substituted by NR⁶R⁷, aryl, hydroxy or C₁₋₄alkoxy, and wherein said ring system is optionally substituted by C₁₋₄alkyl, wherein said C₁₋₄alkyl is optionally substituted by NR⁶R⁷, aryl, hydroxy or C₁₋₄alkoxy;
or unless R³ is selected from: 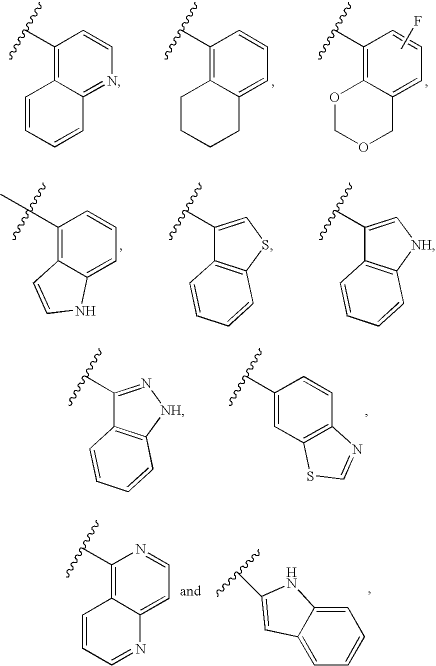
and wherein R³ is optionally substituted by halogen, cyano, nitro, NR⁶R⁷, C₁₋₆alkyl, C₃₋₆cycloalkyl, C₁₋₆alkoxy, C₁₋₆haloalkoxy, aryl or heteroaryl;
or a pharmaceutically acceptable salt thereof, or diastereomers, or enantiomers, or mixtures thereof.

33. A compound according to claim 32, wherein A¹ is N and A² is N.

34. A compound according to claim 32, wherein A¹ is N and A² is CH.

35. (canceled)

36. A compound selected from:
Methyl[(6-{[(cyclohexylmethyl)amino]carbonyl}-5-{[4-(1H-1,2,3-triazol-1-ylmethyl)-1-naphthoyl]amino}pyridin-2-yl)oxy]acetate;
Methyl[(6-{[(cyclobutylmethyl)amino]carbonyl}-5-{[4-(1H-1,2,3-triazol-1-ylmethyl)-1-naphthoyl]amino}pyridin-2-yl)oxy]acetate;
[(6-{[(Cyclobutylmethyl)amino]carbonyl}-5-{[4-(1H-1,2,3-triazol-1-ylmethyl)-1-naphthoyl]amino}pyridin-2-yl)oxy]acetic acid;
6-(2-Amino-2-oxoethoxy)-N-(cyclobutylmethyl)-3-{[4-(1H-1,2,3-triazol-1-ylmethyl)-1-naphthoyl]amino}pyridine-2-carboxamide;
N-(cyclobutylmethyl)-6-[2-(methylamino)-2-oxoethoxy]-3-{[4-(1H-1,2,3-triazol-1-ylmethyl)-1-naphthoyl]amino}pyridine-2-carboxamide;
N-(cyclobutylmethyl)-6-[2-(dimethylamino)-2-oxoethoxy]-3-{[4-(1H-1,2,3-triazol-1-ylmethyl)-1-naphthoyl]amino}pyridine-2-carboxamide;
N-(cyclobutylmethyl)-6-{2-[(2-hydroxyethyl)amino]-2-oxoethoxy}-3-{[4-(1H-1,2,3-triazol-1-ylmethyl)-1-naphthoyl]amino}pyridine-2-carboxamide;
6-{[(Cyclobutylmethyl)amino]carbonyl}-5-{[4-(1H-1,2,3-triazol-1-ylmethyl)-1-naphthoyl]amino}pyridin-2-yl ethanesulfonate;
6-{[(Cyclobutylmethyl)amino]carbonyl}-5-{[4-(1H-1,2,3-triazol-1-ylmethyl)-1-naphthoyl]amino}pyridin-2-yl 3,3,3-trifluoropropane-1-sulfonate;
6-{[(Tetrahydro-2H-pyran-4-ylmethyl)amino]carbonyl}-5-{[4-(1H-1,2,3-triazol-1-ylmethyl)-1-naphthoyl]amino}pyridin-2-yl 3,3,3-trifluoropropane-1-sulfonate;
6-{[(Tetrahydro-2H-pyran-4-ylmethyl)amino]carbonyl}-5-{[4-(1H-1,2,3-triazol-1-ylmethyl)-1-naphthoyl]amino}pyridin-2-yl acetate;
N-(cyclobutylmethyl)-6-(2-hydroxyethoxy)-3-{[4-(1H-1,2,3-triazol-1-ylmethyl)-1-naphthoyl]amino}pyridine-2-carboxamide;
N-(cyclobutylmethyl)-6-[2-(2-hydroxyethoxy)ethoxy]-3-{[4-(1H-1,2,3-triazol-1-ylmethyl)-1-naphthoyl]amino}pyridine-2-carboxamide;
6-(Benzyloxy)-N-(tetrahydro-2H-pyran-4-ylmethyl)-3-{[4-(1H-1,2,3-triazol-1-ylmethyl)-1-naphthoyl]amino}pyridine-2-carboxamide;
3-Benzyl-1-[(4-{[(6-(benzyloxy)-2-{[(tetrahydro-2H-pyran-4-ylmethyl)amino]carbonyl}pyridin-3-yl)amino]carbonyl}-1-naphthyl)methyl]-1H-1,2,3-triazol-3-ium;
N-(cyclobutylmethyl)-6-(pyridin-2-ylmethoxy)-3-{[4-(1H-1,2,3-triazol-1-ylmethyl)-1-naphthoyl]amino}pyridine-2-carboxamide;
N-(cyclobutylmethyl)-3-[(4-{[5-(methoxymethyl)-1H-1,2,3-triazol-1-yl]methyl}-1-naphthoyl)amino]pyridine-2-carboxamide;
N-(cyclobutylmethyl)-3-[(4-{[4-(methoxymethyl)-1H-1,2,3-triazol-1-yl]methyl}-1-naphthoyl)amino]pyridine-2-carboxamide;
N-(cyclobutylmethyl)-3-[(4-{[5-(1-hydroxyethyl)-1H-1,2,3-triazol-1-yl]methyl}-1-naphthoyl)amino]pyridine-2-carboxamide;
N-(cyclobutylmethyl)-3-[(4-{[4-(1-hydroxyethyl)-1H-1,2,3-triazol-1-yl]methyl}-1-naphthoyl)amino]pyridine-2-carboxamide;
3-[(4-{[5-(aminocarbonyl)-1H-1,2,3-triazol-1-yl]methyl}-1-naphthoyl)amino]-N-(cyclobutylmethyl)pyridine-2-carboxamide;
3-[(4-{[4-(aminocarbonyl)-1H-1,2,3-triazol-1-yl]methyl}-1-naphthoyl)amino]-N-(cyclobutylmethyl)pyridine-2-carboxamide;
6-(Aminomethyl)-N-(cyclobutylmethyl)-3-{[4-(1H-1,2,3-triazol-1-ylmethyl)-1-naphthoyl]amino}pyridine-2-carboxamide;
N-(cyclobutylmethyl)-6-(hydroxymethyl)-3-{[4-(1H-1,2,3-triazol-1-ylmethyl)-1-naphthoyl]amino}pyridine-2-carboxamide;
N-(cyclobutylmethyl)-6-{[(methylsulfonyl)amino]methyl}-3-{[4-(1H-1,2,3-triazol-1-ylmethyl)-1-naphthoyl]amino}pyridine-2-carboxamide;
Methyl 6-{[(cyclobutylmethyl)amino]carbonyl}-5-{[4-(1H-1,2,3-triazol-1-yl methyl)-1-naphthoyl]amino}pyridine-2-carboxylate;
N²-(cyclobutylmethyl)-3-{[4-(1H-1,2,3-triazol-1-ylmethyl)-1-naphthoyl]amino}pyridine-2,6-dicarboxamide;
and N-(cyclobutylmethyl)-6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxamide.

37. A compound selected from:
6-(2-Morpholin-4-yl-2-oxo-ethoxy)-3-[(4-[1,2,3]triazol-1-ylmethyl-naphthalene-1-carbonyl)-amino]-pyridine-2-carboxylic acid cyclobutylmethyl-amide;
6-(Benzylcarbamoyl-methoxy)-3-[(4-[1,2,3]triazol-1-ylmethyl-naphthalene-1-carbonyl)-amino]-pyridine-2-carboxylic acid cyclobutylmethyl-amide;
{6-(Cyclobutylmethyl-carbamoyl)-5-[(4-[1,2,3]triazol-1-ylmethyl-naphthalene-1-carbonyl)-amino]-pyridin-2-yloxy}-acetic acid 2,2-dimethyl-propyl ester;
{6-(Cyclobutylmethyl-carbamoyl)-5-[(4-[1,2,3]triazol-1-ylmethyl-naphthalene-1-carbonyl)-amino]-pyridin-2-yloxy}-acetic acid isopropyl ester;
6-Hydroxycarbamoylmethoxy-3-[(4-[1,2,3]triazol-1-ylmethyl-naphthalene-1-carbonyl)-amino]-pyridine-2-carboxylic acid cyclobutylmethyl-amide;
6-(Methoxycarbamoyl-methoxy)-3-[(4-[1,2,3]triazol-1-ylmethyl-naphthalene-1-carbonyl)-amino]-pyridine-2-carboxylic acid cyclobutylmethyl-amide;
{5-[(4-Methyl-naphthalene-1-carbonyl)-amino]-6-[(tetrahydro-pyran-4-ylmethyl)-carbamoyl]-pyridin-2-yloxy}-acetic acid methyl ester;
6-Carbamoylmethoxy-3-[(4-methyl-naphthalene-1-carbonyl)-amino]-pyridine-2-carboxylic acid (tetrahydro-pyran-4-ylmethyl)-amide;
{5-[(4-Methyl-naphthalene-1-carbonyl)-amino]-6-[(tetrahydro-pyran-4-ylmethyl)-carbamoyl]-pyridin-2-yloxy}-acetic acid;
6-(2-Hydroxy-ethoxy)-3-[(4-methyl-naphthalene-1-carbonyl)-amino]-pyridine-2-carboxylic acid (tetrahydro-pyran-4-ylmethyl)-amide;
6-(2-Hydroxy-ethoxy)-3-[(4-methoxymethyl-naphthalene-1-carbonyl)-amino]-pyridine-2-carboxylic acid (tetrahydro-pyran-4-ylmethyl)-amide;
6-Methanesulfonyl-3-[(4-methyl-naphthalene-1-carbonyl)-amino]-pyridine-2-carboxylic acid (tetrahydro-pyran-4-ylmethyl)-amide;
6-Methanesulfinyl-3-[(4-methyl-naphthalene-1-carbonyl)-amino]-pyridine-2-carboxylic acid (tetrahydro-pyran-4-ylmethyl)-amide;
6-[2-(2-Hydroxy-ethoxy)-ethoxy]-3-[(4-[1,2,3]triazol-1-ylmethyl-naphthalene-1-carbonyl)-amino]-pyridine-2-carboxylic acid (tetrahydro-pyran-4-ylmethyl)-amide;
6-methoxy-3-({4-[(4-methylpiperazin-1-yl)methyl]-1-naphthoyl}amino)-N-(tetrahydro-2H-pyran-4-ylmethyl)pyridine-2-carboxamide;
6-methoxy-3-{[4-(morpholin-4-ylmethyl)-1-naphthoyl]amino}-N-(tetrahydro-2H-pyran-4-ylmethyl)pyridine-2-carboxamide;
6-[(ethylamino)sulfonyl]-3-{[4-(methoxymethyl)-1-naphthoyl]amino}-N-(tetrahydro-2H-pyran-4-ylmethyl)pyridine-2-carboxamide;
6-(benzylsulfonyl)-3-{[4-(methoxymethyl)-1-naphthoyl]amino}-N-(tetrahydro-2H-pyran-4-ylmethyl)pyridine-2-carboxamide;
6-(benzylsulfonyl)-3-{[4-(methoxymethyl)-1-naphthoyl]amino}-N-(tetrahydro-2H-pyran-4-ylmethyl)pyridine-2-carboxamide;
6-[(Tetrahydro-2H-pyran-4-ylmethyl)carbamoyl]-5-{[4-(1H-1,2,3-triazol-1-ylmethyl)-1-naphthoyl]amino}pyrazin-2-yl 3,3,3-trifluoropropane-1-sulfonate;
N-(Cyclobutylmethyl)-3-{[4-({5-[(dimethylamino)methyl]-1H-1,2,3-triazol-1-yl}methyl)-1-naphthoyl]amino}pyridine-2-carboxamide;
N-(Cyclobutylmethyl)-3-{[4-({4-[(dimethylamino)methyl]-1H-1,2,3-triazol-1-yl}methyl)-1-naphthoyl]amino}pyridine-2-carboxamide;
N-(Cyclobutylmethyl)-3-[(4-{[4-(trifluoromethyl)-1H-1,2,3-triazol-1-yl]methyl}-1-naphthoyl)amino]pyridine-2-carboxamide;
N-(Cyclobutylmethyl)-3-[(4-{[5-(phenylsulfonyl)-1H-1,2,3-triazol-1-yl]methyl}-1-naphthoyl)amino]pyridine-2-carboxamide;
N-(cyclobutylmethyl)-3-[(4-{[4-(phenylsulfonyl)-1H-1,2,3-triazol-1-yl]methyl}-1-naphthoyl)amino]pyridine-2-carboxamide;
N-(Cyclobutylmethyl)-3-({4-[(4-fluoro-1H-1,2,3-triazol-1-yl)methyl]-1-naphthoyl}amino)pyridine-2-carboxamide;
N-{2-[(cyclobutylmethyl)carbamoyl]pyridin-3-yl}-1-methyl-1H-indole-3-carboxamide;
N-{2-[(cyclobutylmethyl)carbamoyl]pyridin-3-yl}-1-methyl-1H-indole-2-carboxamide;
N-{2-[(cyclobutylmethyl)carbamoyl]pyridin-3-yl}-1H-indole-3-carboxamide;
N-{2-[(cyclobutylmethyl)carbamoyl]-4-methoxyphenyl}quinoline-4-carboxamide;
N-{2-[(cyclobutylmethyl)carbamoyl]-6-methoxypyridin-3-yl}-1-methyl-1H-indazole-3-carboxamide;
3-[(1-benzothien-3-yl-carbonyl)amino]-N-(tetrahydro-2H-pyran-4-ylmethyl)pyridine-2-carboxamide;
3-[(5,6,7,8-tetrahydronaphthalen-1-ylcarbonyl)amino]-N-(tetrahydro-2H-pyran-4-ylmethyl)pyridine-2-carboxamide;
N-{2-[(tetrahydro-2H-pyran-4-ylmethyl)carbamoyl]pyridin-3-yl}-1H-indazole-3-carboxamide;
N-{2-[(tetrahydro-2H-pyran-4-ylmethyl)carbamoyl]pyridin-3-yl}-1H-indole-3-carboxamide;
1-methyl-N-{2-[(tetrahydro-2H-pyran-4-ylmethyl)carbamoyl]pyridin-3-yl}-1H-indole-3-carboxamide;
N-{2-[(tetrahydro-2H-pyran-4-ylmethyl)carbamoyl]pyridin-3-yl}-1,3-benzothiazole-6-carboxamide;
N-{2-[(tetrahydro-2H-pyran-4-ylmethyl)carbamoyl]pyridin-3-yl}-1,6-naphthyridine-5-carboxamide;
3-{[(6-fluoro-4H-1,3-benzodioxin-8-yl)carbonyl]amino}-N-(tetrahydro-2H-pyran-4-ylmethyl)pyridine-2-carboxamide;
N-{2-[(cyclobutylmethyl)carbamoyl]pyridin-3-yl}-1H-indazole-3-carboxamide; and
3-[(4-{[(5-methylisoxazol-3-yl)methoxy]methyl}-1-naphthoyl)amino]-N-(tetrahydro-2H-pyran-4-ylmethyl)pyridine-2-carboxamide.

38. A pharmaceutical composition comprising a compound according to claim 1 as an active ingredient and a pharmaceutically acceptable carrier or diluent.

39-52. (canceled)

53. A method for the treatment of pain, comprising administering a subject in need thereof a pharmaceutically and pharmacologically effective amount of a compound according to claim 1.

54. A compound selected from:
N-(cyclobutylmethyl)-6-hydroxy-3-{[4-(1H-1,2,3-triazol-1-ylmethyl)-1-naphthoyl]amino}pyridine-2-carboxamide;
6-methoxy-N-(tetrahydro-2H-pyran-4-ylmethyl)-3-{[4-(1H-1,2,3-triazol-1-ylmethyl)-1-naphthoyl]amino}pyridine-2-carboxamide;
methyl 3-[(4-{[5-(methoxymethyl)-1H-1,2,3-triazol-1-yl]methyl}-1-naphthoyl)amino]pyridine-2-carboxylate;
methyl 3-[(4-{[4-(methoxymethyl)-1H-1,2,3-triazol-1-yl]methyl}-1-naphthoyl)amino]pyridine-2-carboxylate;
methyl 3-{[4-(azidomethyl)-1-naphthoyl]amino}pyridine-2-carboxylate;
6-Cyano-N-(cyclobutylmethyl)-3-{[4-(1H-1,2,3-triazol-1-ylmethyl)-1-naphthoyl]amino}pyridine-2-carboxamide;
methyl 6-cyano-3-[(4-methyl-1-naphthoyl)amino]pyridine-2-carboxylate;
methyl 6-chloro-3-[(4-methyl-1-naphthoyl)amino]pyridine-2-carboxylate;
6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxylic acid; methyl 6-methoxy-5-[(tetrahydro-2H-pyran-4-ylmethyl)amino]-3-{[4-(1H-1,2,3-triazol-1-ylmethyl)-1-naphthoyl]amino}pyrazine-2-carboxylate;
methyl 5-chloro-6-methoxy-3-{[4-(1H-1,2,3-triazol-1-ylmethyl)-1-naphthoyl]amino}pyrazine-2-carboxylate;
methyl-5-chloro-6-methoxy-3-[(4-methyl-1-naphthoyl)amino]pyrazine-2-carboxylate;
methyl-6-methoxy-3-[(4-methyl-1-naphthoyl)amino]pyrazine-2-carboxylate;
methyl-3-amino-6-methoxypyrazine-2-carboxylate;
3-Amino-6-methoxypyrazine-2-carboxylic acid;
6-Bromo-3-(3-chlorophenyl)pteridine-2,4(1H,3H)-dione;
methyl-3-{[4-(bromomethyl)-1-naphthoyl]amino}pyridine-2-carboxylate;
methyl-3-amino-6-bromopyrazine-2-carboxylate;
6-Hydroxy-3-[(4-methyl-naphthalene-1-carbonyl)-amino]-pyridine-2-carboxylic acid (tetrahydro-pyran-4-ylmethyl)-amide;
6-Hydroxy-N-(tetrahydro-2H-pyran-4-ylmethyl)-3-{[4-(1H-1,2,3-triazol-1-ylmethyl)-1-naphthoyl]amino}pyrazine-2-carboxamide;
3-[(4-Methyl-naphthalene-1-carbonyl)-amino]-6-methylsulfanyl-pyridine-2-carboxylic acid (tetrahydro-pyran-4-ylmethyl)-amide;
6-Chloro-3-[(4-methyl-naphthalene-1-carbonyl)-amino]-pyridine-2-carboxylic acid (tetrahydro-pyran-4-ylmethyl)-amide;
6-chloro-3-{[4-(methoxymethyl)-1-naphthoyl]amino}-N-(tetrahydro-2H-pyran-4-ylmethyl)pyridine-2-carboxamide;
3-{[4-(bromomethyl)-1-naphthoyl]amino}-6-methoxy-N-(tetrahydro-2H-pyran-4-ylmethyl)pyridine-2-carboxamide;
6-(benzylthio)-3-{[4-(methoxymethyl)-1-naphthoyl]amino}-N-(tetrahydro-2H-pyran-4-ylmethyl)pyridine-2-carboxamide;
Methyl-5-methoxy-2-[(quinolin-4-ylcarbonyl)amino]benzoate; and
Methyl-6-methoxy-3-{[(1-methyl-1H-indazol-3-yl)carbonyl]amino}pyridine-2-carboxylate.

55. (canceled)
